(19) United States
(12) Patent Application Publication
(10) Pub. No.: US 2026/0237510 A1
(43) Pub. Date: Aug. 13, 2026

(54) Systems and Methods for Assessment of Tissue Microenvironments and Applications Thereof

(71) Applicants: The Board of Trustees of the Leland Stanford Junior University, Stanford, CA (US); Washington University, St. Louis, MO (US)

(72) Inventors: Aaron M. Newman, San Mateo, CA (US); Wubing Zhang, Stanford, CA (US); Aadel Chaudhuri, St. Louis, MO (US); Erin Brown, Stanford, CA (US)

(73) Assignees: The Board of Trustees of the Leland Stanford Junior University, Stanford, CA (US); Washington University, St. Louis, MO (US)

(21) Appl. No.: 19/440,493

(22) Filed: Jan. 5, 2026

Related U.S. Application Data
(63) Continuation-in-part of application No. PCT/US2025/015148, filed on Feb. 7, 2025.
(60) Provisional application No. 63/550,971, filed on Feb. 7, 2024.
(60) Provisional application No. 63/715,331, filed on Nov. 1, 2024.

(51) Int. Cl. G16H 50/20; C12Q 1/6886; G16B 25/10; G16H 50/30

(52) U.S. Cl. CPC G16H 50/20; C12Q 1/6886; G16B 25/10; G16H 50/30

(57) ABSTRACT

Processes to spatially align single cells to yield a specimen map with single cell resolution are provided. Methods can perform spatial omics on a specimen to yield spatial omics data. The spatial omics data can be used in combination with single cell omics data to assign single cells to spatial coordinates to yield a resolved specimen map.

### CROSS REFERENCE TO RELATED APPLICATIONS

This application is a continuation-in-part of International Patent Application No. PCT/US2025/015148, entitled “Systems and Methods for Assessment of Tissue Microenvironments and Applications Thereof,” filed Feb. 7, 2025, which claims the benefit of U.S. Provisional Patent Appl. No. 63/715,331, entitled “Systems and Methods for Assessment of Tissue Microenvironments and Applications Thereof,” filed Nov. 1, 2024; and U.S. Provisional Patent Appl. No. 63/550,971, entitled “Systems and Methods for Assessment of Tissue Microenvironments and Applications Thereof,” filed Feb. 7, 2024, the disclosures of which are hereby incorporated by reference in their entireties for all purposes.

### STATEMENT REGARDING FEDERALLY SPONSORED RESEARCH OR DEVELOPMENT

This invention was made with Government support under contracts CA255450 and GM142710 awarded by the National Institutes of Health. The Government has certain rights in the invention.

### TECHNICAL FIELD

The disclosure provides description of identifying, assessing, and utilizing spatially defined tissue ecotypes using omics data.

### BACKGROUND

Multicellular ecosystems are fundamental units of tissue organization and key elements of phenotypic variation. In cancer, for example, such ecosystems—arising from immune, stromal, and/or malignant cells—form dynamic signaling hubs that powerfully influence disease progression, immune evasion, and response to therapy. While recent single-cell and bulk genomics studies have revealed critical insights into multicellular ecosystems in the tumor microenvironment, also known as tumor ecotypes, these ecotypes do not consider spatial geographic features within the tumor microenvironment that are important in cellular communication and signaling.

Two major factors hinder the identification and clinical application of spatially resolved ecotypes in cancer. First, spatial ecotypes are challenging to profile using conventional methods, which are either limited in breadth to a modest number of predefined markers (e.g., multiplexed protein imaging), ignore spatial information, or cannot perform integrative analyses across diverse samples, cancer types, and genomic platforms. A second key challenge is the requirement for invasive tumor biopsies to analyze spatial ecotypes in clinical settings. In particular, solid tumor biospecimens are subject to significant sampling bias and generally restricted to a single diagnostic biopsy. While cell-free DNA (cfDNA) has emerged as a promising noninvasive analyte with potential to address these problems, no liquid biopsy assay has been described to date for noninvasive tumor microenvironment (TME) assessment.

### SUMMARY OF THE INVENTION

Systems and methods of the disclosure identify spatial ecotypes in a multicellular system. Spatial omics data can be utilized to identify spatial ecotypes. The spatial ecotypes provide a mean for diagnosing a variety of medical conditions. Based on diagnostics, clinical actions including treatments are performed.

In some aspects, the disclosure provides methods for identifying tissue microenvironments. In certain embodiments, methods comprise acquiring spatial omics data in solid tissue or liquid samples. The data may include a location where applicable, a cell type, and a spatial omic data profile, wherein the spatial omics data profile includes a plurality of cell types, for each solid tissue sample. The spatial omics data are divided into an array of spatial microregions, wherein each microregion is defined by a contiguous spatial area. For each cell type, a cell-type-specific microregion omics data profile is determined. In a preferred instance, the cell-type-specific microregion omic data profile is an aggregate of omic data profiles of cells of the same cell type within the same microregion. A cell-type covariance matrix of cell-type-specific microregion omic data profiles are generated across the array of spatial microregions, and i the cell-type covariance matrices are integrated to yield a sample-level covariance matrix of microregion omic data profiles across the cell types. Preferred methods additionally comprise identifying sample-level microenvironments within the sample-level covariance matrix, wherein each sample-level microenvironment is based on covariance of the microregion omic data profiles.

In some aspects, techniques described herein provide methods in which the cell-type covariance matrices are integrated by fusing the cell-type covariance matrices via a similarity network fusion method.

In some aspects, the techniques described herein relate to a method, wherein identifying sample-level microenvironments includes grouping the microregion omic data profiles via a clustering method.

In some aspects, the techniques described herein relate to a method further including: repeating the steps for a plurality of samples such that a total number samples is greater than two such that sample-level microenvironments are identified in each sample; for each sample-level microenvironment, determine a cell-type-specific microenvironment omic data profile for each cell type of the microenvironment; for each cell type, generating a multi-sample cell-type covariance matrix of cell-type-specific microenvironment omic data profiles across the samples; integrating the multi-sample cell-type covariance matrices to yield a multi-sample-level covariance matrix of cell-type-specific microenvironment omic data profiles across the cell types; identifying conserved microenvironments within the multi-sample-level covariance matrix, wherein each conserved microenvironment is based on covariance of the microenvironment omic data profiles across the samples.

In some aspects, the techniques described herein relate to a method, wherein the plurality of solid tissue samples includes a plurality of tissue types.

In some aspects, the techniques described herein relate to a method, wherein the plurality of tissue types includes a plurality of cancer types.

In some aspects, the techniques described herein relate to a method, wherein the plurality of solid tissue samples includes samples extracted from a plurality of individuals.

In some aspects, the techniques described herein relate to a method, wherein integrating the cell-type covariance matrices includes fusing the multi-sample cell-type covariance matrices via a similarity network fusion method.

In some aspects, the techniques described herein relate to a method, wherein identifying sample-level microenvironments includes grouping the microregion omic data profiles via a clustering method.

In some aspects, the techniques described herein relate to a method further including: training a matrix factorization model to deconvolve the plurality of conserved tissue microenvironments from bulk biomolecule sequencing data of a solid tissue.

In some aspects, the techniques described herein relate to a method further including: determining that one or more conserved tissue microenvironments is indicative of a clinical phenotype; obtaining bulk biomolecule sequencing data of a tissue sample derived from a patient; deconvolving the bulk biomolecule sequencing data and determining that the tissue sample indicates the clinical phenotype based on an abundance of the one or more conserved tissue microenvironments indicative of the clinical phenotype; performing a clinical action upon the patient based on the indication of the clinical phenotype.

In some aspects, the techniques described herein relate to a method, wherein the clinical phenotype is a medical condition capable of being treated by a therapeutic; wherein performing the clinical action includes: administering the therapeutic to the patient.

In some aspects, the techniques described herein relate to a method, wherein the medical condition is cancer and the therapeutic is a chemotherapeutic, a targeted therapeutic, an immune checkpoint inhibitor, or an immunotherapeutic.

In some aspects, the techniques described herein relate to a method, wherein the clinical phenotype is positive responsiveness to a therapeutic for a treatment of a medical condition; wherein performing the clinical action includes: administering the therapeutic to the patient.

In some aspects, the techniques described herein relate to a method, wherein the medical condition is cancer and the therapeutic is a check point inhibitor.

In some aspects, the techniques described herein relate to a method further including: training a binary neural network to detect abundance of one or more conserved tissue microenvironments within a cell-free biomolecule sequencing result.

In some aspects, the techniques described herein relate to a method, further including: determining that one or more conserved tissue microenvironments is indicative of a clinical phenotype; collecting a cell-free biomolecule sample form a patient; sequencing the cell-free biomolecule sample via deep sequencing to obtain a cell-free biomolecule sequencing data result; entering the cell-free biomolecule sequencing data result into the trained binary neural network and inferring that the cell-free biomolecule sample indicates the clinical phenotype based on an abundance of the one or more conserved tissue microenvironments; performing a clinical action upon the patient based on the indication of the clinical phenotype.

In some aspects, the techniques described herein relate to a method, wherein the clinical phenotype is a medical condition capable of being treated by a therapeutic; wherein performing the clinical action includes: administering the therapeutic to the patient.

In some aspects, the techniques described herein relate to a method, wherein the medical condition is cancer and the therapeutic is a chemotherapeutic, a targeted therapeutic, an immune checkpoint inhibitor, or an immunotherapeutic.

In some aspects, the techniques described herein relate to a method, wherein the clinical phenotype is positive responsiveness to a therapeutic for a treatment of a medical condition; wherein performing the clinical action includes: administering the therapeutic to the patient.

In some aspects, the techniques described herein relate to a method, wherein the medical condition is cancer and the therapeutic is a check point inhibitor.

In some aspects, the techniques described herein relate to a method, further including: determining that one or more conserved tissue microenvironments is indicative of a clinical phenotype; repeatedly over a period of time: collecting a cell-free biomolecule sample form a patient; sequencing the cell-free biomolecule sample via deep sequencing to obtain a cell-free biomolecule sequencing data result; entering the cell-free biomolecule sequencing data result into the trained binary neural network to monitor whether the cell-free biomolecule sample indicates the clinical phenotype based on an abundance of the one or more conserved tissue microenvironments.

In some aspects, the techniques described herein relate to a method, wherein the clinical phenotype is progression of a medical condition.

In some aspects, the techniques described herein relate to a method further including: when the progression of the medical condition exceeds a threshold, modify a treatment regimen of a therapeutic; and administer the therapeutic to the patient in accordance with the modified treatment regimen.

In some aspects, the techniques described herein relate to a method, wherein the medical condition is presence of minimal residual disease of cancer and the modified treatment regimen is reinitiating administration of the therapeutic, wherein the therapeutic is a chemotherapeutic, a targeted therapeutic, an immune checkpoint inhibitor, or an immunotherapeutic.

In some aspects, the techniques described herein relate to a method, wherein the clinical phenotype is response to a treatment regimen of a therapeutic.

In some aspects, the techniques described herein relate to a method further including: when the response to therapy indicates a need to modify the treatment regimen of the therapeutic, modifying the treatment regimen of the therapeutic; and administer the therapeutic to the patient in accordance with the modified treatment regimen.

In some aspects, the techniques described herein relate to a method, wherein the response to therapy is a lack of response to the treatment regimen of the therapeutic or an undesired side effect of resulting from the treatment regimen of the therapeutic, wherein modifying the treatment regimen of the therapeutic includes administering an alternative therapeutic to the patient.

In some aspects, the techniques described herein relate to a method, wherein the therapeutic and the alternative therapeutic are each individually a chemotherapeutic, a targeted therapeutic, an immune checkpoint inhibitor, or an immunotherapeutic for the treatment of cancer.

In some aspects, the techniques described herein relate to a method, wherein the response to therapy is a pathologic complete response of a cancer, wherein modifying the treatment regimen of the therapeutic includes terminating administration of the therapeutic to the patient, wherein the therapeutic is a chemotherapeutic, a targeted therapeutic, an immune checkpoint inhibitor, or an immunotherapeutic.

In some aspects, the techniques described herein relate to a method further including: identifying one or more omic-data markers within one or more of the conserved microenvironments.

In some aspects, the techniques described herein relate to a method further including: acquiring single-cell-omic data of a number of solid tissue samples, wherein the number of solid tissue samples is greater than two; assigning cells of the single-cell-omic data to one conserved microenvironment of the identified conserved microenvironments, wherein each cell is assigned based its omic data grouping with the conserved microenvironments via a clustering technique; for each conserved microenvironment, aggregating the omic data of the cells assigned to yield a conserved-microenvironment omic data profile; perform differential analysis comparing each conserved-microenvironment omic data profile with all other conserved-microenvironment omic data profiles; for each putative marker, extract a marker-level log-fold change from the differential analysis; assigning each putative marker to one conserved-microenvironment omic data profile; wherein each putative marker is assigned based on which conserved-microenvironment omic data profile had the putative marker's highest log-fold change as determined by the differential analysis; assigning one or more markers for each conserved-microenvironment omic data profile, wherein each assigned marker has a log-fold change that is one of the highest of the putative markers assigned to that conserved-microenvironment omic data profile.

In some aspects, the techniques described herein relate to a method, wherein each solid tissue sample is a cancer type, wherein the number of solid tissue samples includes at least three cancer types.

In some aspects, the techniques described herein relate to a method further including: determining that one or more conserved tissue microenvironments is indicative of a clinical phenotype; acquiring a omic-data sequencing result of a patient solid tissue sample; assessing the patient's omic-data sequencing result to determine whether the patient's solid tissue sample includes an abundance of the one or more conserved tissue microenvironments that is indicative of the clinical phenotype; wherein the presence of the one or more markers assigned for each conserved-microenvironment omic data profile is utilized to determine the abundance of the one or more conserved tissue microenvironments that is indicative of the clinical phenotype.

In some aspects, the techniques described herein relate to a method, wherein the clinical phenotype is responsiveness to a therapeutic for treating cancer; the method further including: determining that the patient's solid tissue sample includes an abundance of the one or more conserved tissue microenvironments that is indicative of responding to the therapeutic for treating cancer; administering the therapeutic for treating cancer to the patient.

In some aspects, the techniques described herein relate to a method, wherein the therapeutic is a chemotherapeutic, a targeted therapeutic, an immune checkpoint inhibitor, or an immunotherapeutic.

In some aspects, the techniques described herein relate to a method, wherein the omic is one of: transcriptomic, methylomic, epigenomic, or proteomic.

In some aspects, the techniques described herein relate to a diagnostic method for assessing an individual for cancer, including: receiving a collection of a cell-free sample of a patient; and sequencing the cell-free sample to yield a cell-free-sample sequencing result to determine whether an abundance of one or more tumor spatial ecotypes in the patient,

In some aspects, the techniques described herein relate to a method, wherein the abundance of the one or more tumor spatial ecotypes is indicative of a responsiveness to a particular treatment.

In some aspects, the techniques described herein relate to a method further including: determining that the abundance of one or more tumor spatial ecotypes is present in the patient based on the cell-free-sample sequencing result; and administering the to the individual the particular treatment.

In some aspects, the techniques described herein relate to a method further including: determining that the abundance of one or more tumor spatial ecotypes is not present in the patient based on the cell-free-sample sequencing result; and administering the to the individual a treatment that is not the particular treatment.

In some aspects, the techniques described herein relate to a method, wherein determining whether the one or more tumor spatial ecotypes is present in the patient includes: identifying whether a presence one or more markers in the cell-free-sample sequencing result that indicate the abundance of one or more tumor spatial ecotypes is present in the patient.

In some aspects, the techniques described herein relate to a method, wherein the particular treatment includes the administration of an immune checkpoint inhibitor, wherein the one or more markers indicates at least one of: expression of STMN1, TUBB, TYMS or GZMB within CD8 T-cells; expression of TNFRSF4, TNFRSF18, IL2RA, CTLA4, or FOXP3 within CD4 T-cells; expression of CCL8 or ISG15 within macrophages; expression of CXCL8 or MMP1 within fibroblasts; or expression of CEACAM1 or CEBPB within endothelial cells.

In some aspects, the techniques described herein relate to a method, wherein determining whether the one or more tumor spatial ecotypes is present in the patient includes: entering the cell-free-sample sequencing result into a trained binary neural network to infer whether the abundance of the one or more tumor spatial ecotypes is present in the patient.

In some aspects, the techniques described herein relate to a method, wherein the one or more tumor spatial ecotypes is one or more of: Spatial Ecotype 8, Spatial Ecotype 7, and Spatial Ecotype 4.

In some aspects, the techniques described herein relate to a method, wherein the particular treatment includes administration of a chemotherapeutic, a targeted therapeutic, an immune checkpoint inhibitor, or an immunotherapeutic.

In some aspects, the techniques described herein relate to a method, wherein sequencing the cell-free sample includes: methylation-sequencing of cell-free DNA or RNA-sequencing of cell-free RNA.

In some aspects, the techniques described herein relate to a method, wherein the collection of the cell-free sample was collected prior to initiation of a treatment for cancer to assess for presence or progression of cancer within the patient.

In some aspects, the techniques described herein relate to a method, wherein the assessment for presence or progression of cancer within the patient is periodically repeated over a time course to monitor the patient.

In some aspects, the techniques described herein relate to a method, wherein the collection of the cell-free sample was collected after to initiating a treatment of a cancer to assess responsiveness to the treatment.

In some aspects, the techniques described herein relate to a method, wherein the assessment of the responsiveness to the treatment is periodically repeated over a duration of the treatment.

In some aspects, the techniques described herein relate to a method, wherein the collection of the cell-free sample was collected after completing a treatment of a cancer to assess whether minimal residual disease is present within the patient.

In some aspects, the techniques described herein relate to a method, wherein the assessment of whether minimal residual disease is present within the patient is periodically repeated over a time course to monitor the patient.

In some aspects, the techniques described herein relate to a method, wherein the time course is at least one year, at least five years, at least 10 years, or at least 20 years.

In some aspects, the techniques described herein relate to a diagnostic method for assessing an individual for cancer, including: receiving a sample of a solid-tumor biopsy of a patient; extracting nucleic acids from the solid-tumor sample; sequencing the nucleic acids to yield a solid-tumor-sample sequencing result to determine whether an abundance of one or more tumor spatial ecotypes in the patient, wherein the abundance of the one or more tumor spatial ecotypes is: indicative of a responsiveness to a particular treatment, or indicative of a pathology that can be treated by a particular treatment.

In some aspects, the techniques described herein relate to a method further including: determining that the abundance of one or more tumor spatial ecotypes is present in the patient based on the solid-tumor-sample sequencing result; and administering the to the individual the particular treatment.

In some aspects, the techniques described herein relate to a method further including: determining that the abundance of one or more tumor spatial ecotypes is not present in the patient based on the solid-tumor-sample sequencing result; and administering the to the individual a treatment that is not the particular treatment.

In some aspects, the techniques described herein relate to a method, wherein the particular treatment includes administration of a chemotherapeutic, a targeted therapeutic, an immune checkpoint inhibitor, or an immunotherapeutic.

In some aspects, the techniques described herein relate to a method, wherein determining whether the one or more tumor spatial ecotypes is present in the patient includes: identifying whether a presence one or more markers in the solid-tumor-sample sequencing result that indicate the abundance of one or more tumor spatial ecotypes is present in the patient.

In some aspects, the techniques described herein relate to a method, wherein the particular treatment includes the administration of an immune checkpoint inhibitor, wherein the one or more markers indicates at least one of: expression of STMN1, TUBB, TYMS or GZMB within CD8 T-cells; expression of TNFRSF4, TNFRSF18, IL2RA, CTLA4, or FOXP3 within CD4 T-cells; expression of CCL8 or ISG15 within macrophages; expression of CXCL8 or MMP1 within fibroblasts; or expression of CEACAM1 or CEBPB within endothelial cells.

In some aspects, the techniques described herein relate to a method, wherein determining whether the one or more tumor spatial ecotypes is present in the patient includes: entering the solid-tumor-sample sequencing result into a matrix factorization model to deconvolve the sequencing result into abundances of tumor spatial ecotypes to determine whether the abundance of one or more tumor spatial ecotypes is present in the patient.

In some aspects, the techniques described herein relate to a method, wherein the one or more tumor spatial ecotypes is one or more of: Spatial Ecotype 8, Spatial Ecotype 7, and Spatial Ecotype 4.

In some aspects, the techniques described herein relate to a method, wherein sequencing the cell-free sample includes: methylation-sequencing of cellular DNA or RNA-sequencing of cellular RNA.

In some aspects, the techniques described herein relate to a method, wherein the biopsy of the solid-tumor sample was collected prior to initiation of a treatment for cancer.

In some aspects, the techniques described herein relate to a method, wherein the biopsy of the solid-tumor sample was collected after to initiating a treatment of a cancer to assess whether the treatment is to be modified.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides an example of a computational method for prediction whether a region transcriptomic data is derived from tumor or stroma.

FIG. 2 provides an example of a computational method for yielding spatial clusters within a sample using single-cell spatial omic data.

FIG. 3 provides an example of a computational method for yielding spatial ecosystems across a collection of samples using spatial clusters of microregions.

FIG. 4 provides an example of a computational method for training a matrix factorization model to deconvolve spatial ecotypes from bulk tissue omic data.

FIG. 5 provides an example of computational method of utilizing a trained matrix factorization model to deconvolve spatial ecotypes from a sample of bulk tissue omic data and various downstream applications.

FIG. 6 provides an example of a computational method for training a binary neural network to detect abundance of spatial ecotypes within the cell-free-sourced data.

FIG. 7 provides an example of computational method of utilizing a trained binary neural network to detect abundance of spatial ecotypes within the cell-free-sourced data and various downstream applications.

FIG. 8 provides an example of computing systems for spatial omic assessments.

FIGS. 9A-9C provide multimodal profiling of spatial ecotypes in human cancer. FIG. 9A, Schematic description of the study. Top: Discovery and clinical characterization of spatially co-localized cell states in human tumors, termed spatial ecotypes (SEs). Bottom: Recovery of SEs in plasma cell-free DNA and the use of noninvasive SE profiling for immunotherapy response assessment. FIG. 9B, Compendium of human tumor ST samples (left) and single-cell expression profiles (right) curated and analyzed in this work. Inner and outer rings denote platform and cancer type proportions, respectively. FIG. 9C, Major cell types and key geographic regions in representative breast cancer specimens profiled by Vizgen MERSCOPE (left, n=365,811 cells) and 10× Genomics Visium (right; n=16,860 cells). The latter was integrated with scRNA-seq data using CytoSPACE, resulting in a single-cell reconstructed ST specimen. For additional details, including the annotation of tumor and adjacent stromal regions.

FIGS. 10A-10D provide data inventory and workflow for distinguishing tumor from adjacent stroma. FIG. 10A, Similar to FIG. 9B but stratified by cancer type. FIG. 10B, Workflow for classifying tumor and stroma regions from bulk ST data (10× Genomics Visium and legacy ST) using logistic regression (LR). Four key features were used for training and prediction: the inferred fraction of cancer-cell-of-origin cells per spot, the number of expressed genes per spot, and the mean of each feature when considering one-hop (i.e., immediate) neighbors. FIG. 10C, Box plot showing the performance of the LR model (panel b) for classifying tumor from adjacent stromal spots in 29 ST samples (eight 10× Genomics Visium and 21 legacy ST) annotated by pathologists. Performance was evaluated using leave-one-cancer-out cross-validation, with the area under receiver operating characteristic curve (AUC) computed for each sample in the held-out cancer type. The box center lines, bounds of the box, and whiskers denote medians, 1^{st} and 3^{rd} quartiles, and minimum and maximum values within 1.5×IQR (interquartile range) of the box limits, respectively. The median of medians (AUC=0.82) is indicated by a red dashed line. FIG. 10D, Scatter plot showing gene expression differences (log₂ fold change) between tumor and adjacent stromal regions annotated by the LR model (y-axis) versus those annotated by pathologists (x-axis) in paired samples. In all cases, LR predictions reflect cancer types held out from training. Performance was determined by Pearson correlation and linear regression. P-values were determined with a two-sided t test.

FIGS. 11A-11C provide pan-cancer spatial phenotypes in tumor and adjacent stromal tissue. FIG. 11A, Heat maps depicting pan-cancer gene expression variation between tumor and adjacent stromal regions for 8 TME cell types, 10 malignancies, and 121 ST tumor samples covering four platforms. Genes satisfying differential expression requirements in the discovery cohort (Q<0.05, median log₂ fold change [FC] across samples >0.05), with a maximum of 100 genes per compartment, are shown. The top 10 gene symbols per compartment are highlighted. All ST datasets were reconstructed using scRNA-seq profiles of matching malignancies using CytoSPACE. FIG. 11B, Same as panel a but showing spatial expression programs that stratify tumor and adjacent stroma independent of TME cell type (n=9), malignancy (n=10), or ST platform (n=4). Genes with asterisks (PKM and FOS) denote the top markers in the Visium discovery cohort that are also covered by MERSCOPE (see also panel c). FIG. 11C, Spatial polarization of PKM and FOS expression in tumor and adjacent stromal regions in a representative liver cancer specimen profiled by MERSCOPE (‘Liver 2’). Left: Annotated cell types, tumor/stromal regions, and specimen-wide expression of PKM and FOS. Right: Representative microregions (50 μm²) showing PKM and FOS mRNA transcripts in diverse cell types (colored as in b).

FIGS. 12A-12E provide extended analysis of spatially variant genes and cell states. FIG. 12A, Scatter plots showing cross-platform consistency of differentially expressed genes between tumor and adjacent stroma in representative TME cell types, comparing bulk ST data (10× Visium) reconstructed with scRNA-seq data using CytoSPACE¹ (x-axis) versus single-cell ST data (MERSCOPE) (y-axis). Performance was determined by Pearson correlation and linear regression, with 95% confidence intervals shown. P-values were determined with a two-sided t test. Colors reflect the mean of both axes, with orange and purple denoting tumor-associated and stromal-associated genes, respectively. FIG. 12B, Same as FIG. 11a but shown for plasma cells. FIG. 12B, Box plots showing the spatial polarization of previously defined tumor-associated cell states. Normalized enrichment scores (NES) (x-axes) capture the statistical skewing of the top 50 marker genes of each cell state within an ordered expression vector of log 2 fold changes between tumor and stroma, balanced by cancer type. NES values were determined by pre-ranked gene set enrichment analysis (GSEA). The box center lines, bounds of the box, and whiskers denote medians, 1^{st} and 3^{rd} quartiles, and minimum and maximum values within 1.5×IQR (interquartile range) of the box limits, respectively. FIG. 12D, Heat map depicting NES values of hallmark pathways in tumor versus adjacent stroma for each evaluable ST platform. Here, gene sets were applied to an ordered expression vector of log 2 fold changes between tumor and stroma, balanced by TME cell type and cancer type. FIG. 12E, Same as FIG. 11C but shown for ‘Colon 2’. Prior to analysis, CytoSPACE was used to reconstitute all ST samples with scRNA-seq data in panel a (x-axis) and panels 12B-12D.

FIGS. 13A-13G provide geospatial map of multicellular programs across cancers. FIG. 13A, Graphical summary of Spatial EcoTyper applied to a melanoma specimen profiled by MERSCOPE, highlighting three key steps: identification of spatial gene expression profiles (sGEPs) for each cell type from a regular grid of n microregions; cross-comparison of sGEPs to define a covariance matrix (n×n) for each cell type; and fusion of covariance matrices into a single spatial embedding (n×n covariance matrix). To distinguish individual cell types within each spatial microregion by color (right), a small amount of jitter was used for clarity. UMAP projections of spatial embeddings generated for five tumor specimens profiled by MERSCOPE (four carcinomas and one melanoma). Each point denotes an individual microregion colored by physical distance to the tumor-stoma interface. FIG. 13C, Same as panel b but colored by multicellular spatial communities, termed spatial ecotypes (SEs), identified from tumor samples in panel b. FIG. 13D, Heat map showing a fused spatial covariance matrix from five tumor specimens (panel b), representing 41 k individual spatial microregions grouped into 892 microregion clusters (rows and columns), ordered by SEs (n=9). The similarity index is a measure of microregion similarity, accounting for the balanced contribution of cell-type-specific spatial GEPs across samples. SEs were labeled SE1 to SE9 according to their average physical distance to the tumor margin. Network diagrams of SE-specific cell states. Thicker edges denote more significant spatial co-localization between cell states. FIG. 13F, Spatial distribution of SEs and other features in melanoma and breast tumor specimens profiled by MERSCOPE. From left to right, a global view of spatial ecotypes and magnified views (1 mm²) of SE composition (colored as in d), geographic features, and cell types (colored as in panels d, b, and e, respectively). FIG. 13G, Heat maps showing the relative expression of SE-specific cell state markers across 10 cancer types (nine carcinomas and melanoma) profiled by scRNA-seq.

FIG. 14 provides a framework for spatial ecotype discovery. Graphical summary of the Spatial EcoTyper “discovery module,” illustrating its application to a single tumor specimen (steps 1 to 3) and to multiple tumor specimens (steps 4 to 6). Steps 1 and 2 are identical to FIG. 13A, showing the creation of a spatial embedding matrix (n×n covariance matrix) from a melanoma sample profiled by MERSCOPE. In step 3, Louvain clustering is applied to the spatial embedding matrix, followed by computation of average expression within each cluster, resulting in an g_{c}×k expression matrix for each cell type c, with g, genes (rows) discriminating k clusters (columns). Next, steps 1 to 3 are repeated on different tumor specimens (in this case, five). The resulting matrices are concatenated into a single GEP matrix for each cell type c, denoted E^{c}, with rows representing common genes and columns representing the union of clusters (for a total of K clusters). In step 5, the columns of E^{c} are subjected to a cross-comparison, yielding a K×K spatial covariance matrix for each cell type. In step 6, covariance matrices from step 5 are fused using SNF. NMF is subsequently applied to define robust spatial clusters termed SEs.

FIGS. 15A-15E provides Benchmarking of Spatial EcoTyper. FIG. 15A, Spatial ecotype clusters defined by Spatial EcoTyper and previous methods in a representative melanoma specimen profiled by MERSCOPE (‘Melanoma 1’;). FIG. 15B, Comparison of methods for identifying spatial ecotypes in tumor samples from the Spatial EcoTyper discovery and validation cohorts, applied to each tumor sample individually. Left: Bubble plot showing the relative performance of each method for identifying spatial ecotype clusters using three metrics: (i) ‘spatial co-localization’, which captures the local contiguity of cells within each cluster, (ii) ‘cell type mixing’, which captures the diversity of cell types per cluster (higher scores denote more cell types), and (iii) ‘mean silhouette width’, which captures, for each cell type, the degree of gene expression profile (GEP) separation between clusters and the degree of GEP similarity within the same cluster (higher scores denote greater cluster separation and compactness). Bubble sizes reflect the relative ranking of methods based on each metric. Each quantity was first averaged across clusters within each sample, converted to rank space, and then averaged across samples. Right: Box plot aggregating the three metrics from each tumor sample by geometric mean of their ranks. FIG. 15C, UMAP embeddings showing the relative ability of selected methods to integrate two single-cell ST samples of the same cancer type (‘Melanoma 1’ and ‘Melanoma 2’), with cells colored by samples (top), tumor and adjacent stromal regions (center), and cell types (bottom). In the Spatial EcoTyper UMAP, a small amount of jitter was applied to display individual cells within each spatial cluster. FIG. 15D, Scatter plot comparing Spatial EcoTyper against all methods in the benchmarking analysis that enable sample integration, showing their relative performance for identifying spatial ecotypes conserved across ‘Melanoma 1’ and ‘Melanoma 2’. Performance was assessed using metrics that quantify the degree of cell type mixing (x-axis) and sample mixing (y-axis), averaged across identified spatial ecotypes. FIG. 15E, Scatter plot summarizing the performance of each method for identifying spatial ecotypes, combining single-sample analysis (panel b) and integrative analysis (panel d). Integration performance is computed as the geometric mean of cell type mixing and sample mixing metrics (panel d) in rank space.

FIGS. 16A-16E provide robustness of spatial ecotype discovery. FIG. 16A, Composition of MERSCOPE specimens used for SE discovery and validation. Only samples with more than 5% TME cells derived from tumor or adjacent stromal regions were include. FIG. 16B, Robustness of spatial phenotypic embeddings to microregions of diverse radii, related to FIG. 13B. Here, Spatial EcoTyper was applied to a melanoma specimen profiled by MERSCOPE (‘Melanoma 1’, Supplementary Table 7) (left panel) to create spatial embeddings as illustrated in FIG. 13A, but where the microregion radius was varied (center panel). Each point in the embedding denotes an individual microregion. Right: Slingshot⁴ was applied to each embedding to create a one-dimensional “pseudospace” trajectory of each microregion using principal curve analysis. Linearity was then quantified as the Spearman correlation between pseudospace and the physical distance of each microregion to the tumor margin, weighted by the number of cells in each compartment (tumor and adjacent stroma). A radius of 50 μm, reflecting a balance between peak linearity and smaller radius, was selected for subsequent analysis. FIG. 16C, Analysis of the linear relationship between the organization of sample-specific spatial embeddings in FIG. 13B and physical distance to the tumor margin. Linearity was quantified as in panel b. Statistical significance was calculated with a two-sided t-test. FIG. 16D, Cophenetic coefficient plot for NMF applied to the fused spatial covariance matrix generated by Spatial EcoTyper. NMF was initialized across a range of cluster numbers. The red arrow indicates the cluster number selected for subsequent analysis, corresponding to the region of the graph with the largest subsequent drop. FIG. 16E, Robustness of SEs to Louvain clustering at different resolutions. The average adjusted rand index (ARI) comparing clusters at each resolution (x-axis) with the results obtained at other resolutions is shown (Methods). The red arrow marks the resolution used for SE discovery in the study.

FIGS. 17A-17B provide spatial ecotype localization and cell state composition. FIG. 17A, Box plot showing the average physical distance of each SE to the tumor margin across tumor samples in the discovery cohort. The box center lines, bounds of the box, and whiskers denote medians, 1^{st} and 3^{rd} quartiles, and minimum and maximum values within 1.5×IQR (interquartile range) of the box limits, respectively. FIG. 17B, Identification of SE-specific cell states via supervised NMF and leave-one-sample-out cross-validation (LOOCV). Left: Schema of the approach. Right: Heat map showing the normalized F1 score for each cell state in each SE, reflecting the specificity of each cell state for the SE from which it was derived, as determined by LOOCV.

FIGS. 18A-18K provide validation of spatial ecotypes. FIG. 18A, Workflow for recovering and validating SEs using supervised NMF models trained on SE-enriched cell states. Cell states were assigned to single cells (MERSCOPE, scRNA-seq) and deconvolved from bulk ST spots (10× Genomics Visium, legacy ST) to determine ecotype membership and impute relative abundances. FIG. 18B-18E, Scatter plots showing the consistency between predicted and expected (panel a) distances of SEs to the tumor margin, shown for the discovery cohort via LOOCV (FIG. 18B) and in held-out tumor samples profiled by MERSCOPE (FIG. 18C), 10× Genomics Visium (FIG. 18D), and legacy ST (FIG. 18E). Each cell (MERSCOPE) or spatial spot (bulk ST) was assigned to a unique SE or to a null class if insufficient SE signal was detected. The closest Euclidean distance between the tumor/stroma margin and each SE location was averaged by SE and balanced by cancer type. Concordance was determined by Pearson correlation and linear regression, with 95% confidence intervals shown. P-values were determined with a two-sided t test. FIG. 18F-18J, Heat maps showing the co-association of SE-enriched cell states both in the discovery cohort (LOOCV) FIG. 18F) and in held-out tumor samples profiled by MERSCOPE (FIG. 18G), 10× Genomics Visium (FIG. 18H), legacy ST (FIG. 18I), or scRNA-seq (FIG. 18J). Given the high granularity of MERSCOPE data, we calculated a spatial co-localization index in panels f and g, defined as the background-normalized probability of identifying cell states from the same SE within a 50 μm radius by random chance. For bulk ST and scRNA-seq data, we predicted cell-state abundances within each spatial spot and sample, respectively, then determined a co-association index for each cell-state pair by pairwise Pearson correlation. The latter was determined within-sample for ST data and across-sample for scRNA-seq data. All co-association indices were subsequently balanced across cancer types. Statistical significance was determined by permutation testing and meta-statistics, as described in. FIG. 18K, Heat map depicting the overlap between spatial ecotypes and carcinoma ecotypes (CEs) in scRNA-seq data, with the latter ordered by a previously determined spatial aggregation score (Moran's I). The overlap index was computed as the fraction of cells within SE i that are jointly assigned to CE j, normalized by the fraction expected by random chance and expressed as a z-score averaged across 10 evaluable cancer types. *P<0.05; **P<0.01; ***P<0.001; ****P<0.0001.

FIGS. 19A-19E provide Biological and clinical characteristics of spatial ecotypes. FIGS. 19A-19C, Performance of SE deconvolution applied to pseudo-bulk and real bulk RNA-seq profiles of human tumors. FIG. 19A, Box plots showing Pearson correlation coefficients between predicted and expected SE proportions in pseudo-bulk tumors of cancer types held-out from training. Results for 10 malignancies and 1,000 pseudo-bulk tumors from 10 scRNA-seq datasets are shown. FIG. 19B, Scatter plots showing concordance between predicted and expected SE proportions, with the former deconvolved from real bulk RNA-seq data of melanomas (n=4) and colorectal tumors (n=4) and the latter determined from paired scRNA-seq data. Both assays were performed on the same cell suspensions to minimize bias. Performance was determined by Pearson correlation and linear regression, with 95% confidence intervals shown. P-values were determined with a two-sided t test. FIG. 19C, Box plots comparing Pearson correlation coefficients from panel a (calculated across cancers and data points), panel b, and the same analysis as panel b but performed on bulk RNA-seq of intact (non-dissociated) tumors. Statistical significance was determined using a two-sided paired Wilcoxon test. *P<0.05. FIG. 19D, Biological and clinical characteristics of SE levels deconvolved from 7,076 bulk RNA-seq profiles spanning 17 TCGA cancer types (16 carcinomas and skin cutaneous melanoma). Top: Bar plot showing associations between higher inferred SE abundances and overall survival (expressed as −log₁₀ P values), integrated across cancer types using meta-z statistics. Blue and red bars denote associations with favorable and adverse outcomes, respectively. Center Same as the top but shown for individual cancer types without integration. Bottom: Biological features enriched or depleted in each SE, determined by Spearman correlation between inferred SE abundances and each feature. FIG. 19E, Heat map depicting the differential capacity of 41 features (rows), including inferred SE levels, to predict ICI response both within and across 14 bulk RNA-seq datasets of tumors stratified by ICI therapy (columns). Rows are organized top to bottom by statistical significance, from features that most strongly predict benefit to those that most strongly predict resistance; the top 10 features are indicated for each outcome. ICI, immune checkpoint inhibitor. In a and c, the box center lines, bounds of the box, and whiskers denote medians, 1st and 3rd quartiles, and minimum and maximum values within 1.5×IQR (interquartile range) of the box limits, respectively.

FIGS. 20A-20C provide Bulk RNA-seq deconvolution of spatial ecotypes. FIG. 20A, Scatter plots showing the concordance between predicted and expected SE proportions in pseudo-bulk tumor samples (n=1,000). Each cancer type was held out from training in an LOOCV setting, with performance evaluated using Pearson correlation and linear regression. P-values were calculated using a two-sided t test. FIG. 20B, Similar to a, but displaying cross-SE Pearson correlations as a heat map. Each cell in the heat map represents the correlation between predicted and expected proportions for different SEs across the pseudo-bulk tumor samples (n=1,000). FIG. 20C, Workflow for assessing deconvolution performance of Spatial EcoTyper using paired bulk RNA-seq and single-cell RNA-seq data from melanoma and colorectal tumor specimens. SEs were deconvolved from bulk RNA-seq data to estimate SE abundances, while cell states were assigned to single cells to determine ecotype membership and infer relative abundances from scRNA-seq data, related to FIGS. 19B, 19C.

FIGS. 21A-21C provide development and technical assessment of Liquid EcoTyper. FIG. 21A, Workflow of Liquid EcoTyper, a deep learning model designed to predict relative SE levels from DNA methylation profiles of tumor or plasma cfDNA samples. The model utilizes CpG methylation profiles as input, which are processed through a binary network module⁶ to identify informative CpG sets. For each identified CpG set, the average methylation level is computed and subsequently transformed into continuous outputs representing relative SE levels. FIG. 21B, Schematic illustrating the simulation of plasma cfDNA for the development of Liquid EcoTyper. Triplets of healthy plasma methylation profiles were first combined with noise, followed by mixing with melanoma methylation profiles to simulate melanoma plasma methylation. FIG. 21C, Workflow for the technical evaluation of Liquid EcoTyper. Relative SE levels were inferred from: (i) held-out simulated plasma cfDNA data and (ii) methylation data from paired tumor, plasma, and PBMC samples. These inferred SE levels were then compared across different data modalities or analytes to assess performance.

FIGS. 22A-22I provide Noninvasive early assessment of immunotherapy response with spatial ecotypes. FIG. 22A, Heat map showing Spearman correlation coefficients between predicted and expected SE levels in 115 held out simulated plasma cfDNA samples, with the former determined from methylation data using Liquid EcoTyper. FIG. 22B. Cross-compartment comparisons of SE profiling from whole-genome EM-seq profiles of paired tumor and plasma samples from 10 patients with metastatic melanoma. Linearity was determined using Pearson and Spearman correlation and linear regression (with a 95% confidence band shown). SE levels were determined with Liquid EcoTyper. FIG. 22C, Box plots comparing Spearman correlation coefficients from the same analyses as panel b but performed on seven samples with paired tumor, plasma and PBMC samples. A two-sided paired Wilcoxon test was used to assess statistical significance between boxes (top). A one-sample t-test was used to determine whether the correlations within each box are significantly different from zero (bottom). ****P<0.0001; ns, not significant. FIGS. 22D-22I, Application of noninvasive SE profiling to ICI response prediction in patients with metastatic melanoma (n=79). FIG. 22D, Scatter plot showing ICI response associations, expressed as z-scores, between SE levels inferred from (i) whole-genome EM-seq data of pretreatment plasma cfDNA from patients with advanced melanoma (n=79) and (ii) bulk RNA-seq data of pretreatment tumors from patients with advanced melanoma (n=366). Positive and negative z-scores indicate that higher SE levels are associated with resistance and response to ICIs, respectively. Linearity was determined by Pearson correlation and linear regression (with a 95% confidence band shown), and statistical significance was determined by a two-sided t test. FIG. 22E, Heat map of melanoma patients profiled by whole-genome EM-seq in this study (n=79) showing, from top to bottom, patient clinical characteristics, pretreatment SE7, SE8, and SE4 levels determined by Liquid EcoTyper, and pretreatment ctDNA levels determined by AVENIO (n=62 evaluable patients). FIG. 22F, Box plots showing inferred SE7 levels in pretreatment plasma stratified by ICI response and shown for distinct ICI therapies. Statistical difference was determined using a two-sided Wilcoxon rank sum test. FIG. 22G, Kaplan-Meier plot showing differences in progression free survival of melanoma patients (n=79) dichotomized into high and low groups based on the median of inferred SE7 levels in pretreatment plasma. Statistical significance was determined by a two-sided log-rank test. HR, hazard ratio. 95% HR confidence intervals are shown in brackets. FIG. 22H, Same as panel f but for SE4. FIG. 22I, Same as panel g but for SE4. In panel c, f, and h, the box center lines, bounds of the box, and whiskers denote medians, 1st and 3rd quartiles, and minimum and maximum values within 1.5×IQR (interquartile range) of the box limits, respectively. cfDNA, cell-free DNA; ctDNA, circulating tumor DNA; ICI, immune checkpoint inhibitor; DCB, durable clinical benefit; NDB, no durable benefit; AF, allele frequency; SNV, single nucleotide variant.

FIGS. 23A-23K provide Noninvasive early assessment of patient survival and immunotherapy response with liquid SEs. FIG. 23A, Box plots showing inferred SE8 levels in pretreatment plasma from patients with melanoma stratified by ICI response and shown for distinct ICI therapies. Statistical significance was determined using a two-sided Wilcoxon rank sum test. FIG. 23B, Kaplan-Meier plots showing differences in progression free survival (left) and overall survival (right) of melanoma patients dichotomized into high and low groups based on the median of inferred SE8 levels in pretreatment plasma. FIG. 23D, Same as the right panel of b, but for SE4 and SE7. FIG. 23D, Same as the left panel of b, but for SE7, across different ICI therapies. FIG. 23E, Same as d, but for SE4. In b-e, statistical significance was determined by a two-sided log-rank test. Hazard ratios (HRs) with 95% confidence intervals (Cis) are shown. FIG. 23F-23H, Box plots showing inferred SE7 (FIG. 23F), SE8 (FIG. 23G), and SE4 (FIG. 23H) levels in pretreatment plasma stratified by ICI response across datasets from different institutes. The area under the receiver operating characteristic (ROC) curve (AUC) was calculated within each dataset. FIG. 23I, Dot plot showing the optimal thresholds for classifying ICI response across different datasets. Thresholds were determined by maximizing Youden's J statistic⁷ from the ROC curves. Statistical significance was calculated with a two-sided paired Wilcoxon test. ns, not significant. FIG. 23J, Bar plot comparing the association between SEs and ctDNA with ICI response (left) and overall survival (right). Z-scores were derived from a two-sided Wilcoxon rank-sum test for response association (left) and univariate Cox regression for survival association (right) using AVENIO ctDNA data from 62 melanoma patients. FIG. 23K, Forest plots showing the overall survival association of SE7 (left), SE8 (center), and SE4 (right), adjusted for covariates including baseline ctDNA level (AVENIO), melanoma subtypes, sex, age, and treatment, in a multivariate Cox regression model (Supplementary Table 22). Statistical significance was determined by a two-sided Wald test. 95% HR confidence intervals are shown in brackets. Squares at the midpoints and the accompanying bars denote the natural logarithm of HRs and their 95% confidence intervals, respectively. In a and f-h, the box center lines, bounds of the box, and whiskers denote medians, 1^{st} and 3^{rd} quartiles, and minimum and maximum values within 1.5×IQR (interquartile range) of the box limits, respectively. ctDNA, circulating tumor DNA; SNV, single nucleotide variant; ICI, immune checkpoint inhibitor; DCB, durable clinical benefit; NDB, no durable benefit. *P<0.05; **P<0.01; ***P<0.001.

FIG. 24 is a table of biological processes enriched in SE markers.

FIG. 25 shows a list of consensus marker that were generalizable to sing cell scale ST data and associated with distinct functions.

### DETAILED DESCRIPTION

Turning now to the drawings and data, systems and methods to identify and utilize special ecosystems of solid multicellular systems are provided. In the various embodiments of the systems and methods, spatial omic data is utilized to identify microregions within a sample spatial ecotypes (SEs) across samples. An SE is a regionalized collection of cell types, each cell type within the regionalized collection sharing a similar cell state, that are conserved across a set of samples. In other words, an SE is defined by the cell states of the cell types that are conserved within a proximal area of a mulicelluar system. Once spatial ecotypes of a tissue type are defined, systems and methods can utilize omic data from a variety of sources to identify or infer SEs within a solid multicellular system. The systems and methods can further utilize inferred SEs in a number of practical applications, such as diagnostics and clinical assessments that indicate administering a particular clinical action or a particular treatment. The various systems and methods provide improvements various fields of clinical assessment, such as (for example) predicting patient outcome, determining benefit of a therapy, monitoring progression of a clinical phenotype, and monitoring effect of a treatment.

The various systems and methods refer to cell types and cell states. The term “cell type” is to refer to a particular label of a cell that defines an overall function. For example, cell types can refer to various immune cells (e.g., macrophages, CD4 T-cells, CD 8 T-cells, B-cells, etc.) or to various cells of an organ system (e.g., cardiomyocytes, pericytes, myeloid cells, fibroblasts, adipocytes, endothelial cells, etc.). The term “cell state” is to refer to a particular omic profile of a cell type, which can be influenced by various factors such as maturity, neighboring cell contact, local signaling, and state of activation. As detailed herein, spatial ecotypes of a tissue can be defined by a regional composition of cell types having a particular set of one or more cell states.

The various systems and methods can be applied a variety of omics. The term “omics” is to be understood any of a variety of substantially comprehensive molecular analyses of a cell. In some implementations, “omics” refers to transcriptomics, genomics, epigenomics, methylomics, proteomics, and metabolomics. Further, as is understood in the field, any and all these omics can be utilized for spatial analysis and thus the systems and methods can be adapted to the specific parameters for performing such analysis. Generally, when any of a particular set of omics can delineate one cell state from another cell state, the systems and methods as described herein can be applied, especially transcriptomics, epigenomics, methylomics, and proteomics. For more details on spatial transcriptomics, see, e.g., M. Asp, J. Bergenstrahle, and J. Lundeberg, Bioessays. 2020 October; 42(10):e1900221; and P. L. Stahl, et al., Science. 2016 Jul. 1; 353(6294):78-82; the disclosures of which are each incorporated herein by reference. For more details on spatial genomics, see, e.g., T. Zhao, et al., Nature. 2022 January; 601(7891):85-91; and R. U. Sheth, et al., Nat Biotechnol. 2019 August; 37(8):877-883; the disclosures of which are incorporated herein by reference. For more details on spatial epigenomics, see, e.g., T. Lu, et al., Cell. 2022 Nov. 10; 185(23):4448-4464.e17, the disclosure of which is incorporated herein by reference. For more details on spatial methylomics, see, e.g., N. Loyfer, et al., Nature. 2023 January; 613(7943):355-364, the disclosure of which is incorporated herein by reference. For more details on spatial proteomics, see, e.g., E. Lundberg and G. H. H. Borner, Nat Rev Mol Cell Biol. 2019 May; 20(5):285-302, the disclosure of which is incorporated herein by reference. For more details on spatial metabolomics, see, e.g., L. R. Conroy, et al., Nat Commun. 2023 May 13; 14(1):2759, the disclosure of which is incorporated herein by reference. Further, it should be understood that various omics can be combined for spatial analysis, for instance, as described in D. Zhang, et al., Nature. 2023 April; 616(7955):113-122, the disclosure of which is incorporated herein by reference.

Two major factors currently hinder the identification and clinical application of spatially resolved ecotypes in tissues such as cancer. First, spatial ecotypes (SEs) are challenging to profile using existing methods, which are either limited in breadth to a modest number of predefined markers (e.g., multiplexed protein imaging), ignore spatial information, or cannot perform integrative analyses across diverse samples, phenotypic types (e.g., cancer types), and genomic platforms. A second key challenge is the is the difficulty to analyze SEs within clinical settings. In particular, solid tissue biospecimens (e.g., tumor biopsy) are subject to significant sampling bias and generally restricted to a single diagnostic biopsy, as obtaining such biopsies can be painful and burdensome on the individual. While cell-free DNA (cfDNA) has emerged as a promising noninvasive analyte with potential to address these problems, no liquid biopsy assay has been described to date that can assess spatial ecotypes of a solid tissue.

The innovative systems and methods described herein introduce a new multimodal framework for solid and liquid profiling of SEs in the TME. These systems and methods combine data fusion, statistical learning, and deep learning to overcome critical barriers in both the detection and recovery of SEs across genomic platforms and bodily compartments. In many embodiments, systems and methods utilize omic data collected from a tissue to identify and quantify SEs within that tissue. In various embodiments, the systems and methods can utilize one of: spatial omic data, bulk omic data, or cell-free omic data to identify SEs present within a tissue. In several embodiments, a trained computational matrix factorization model is utilized to infer SEs from bulk tissue omic data. In many embodiments, a trained binary model is utilized to infer SEs of a tissue from cell-free omic data. And in several embodiments, identified or inferred SEs are utilized within statistical assessments and/or regression models for various practical applications such as (for example) diagnostics for determining treatment response.

### Spatial Assessment of Solid Tissue

Several embodiments are directed to training and utilizing a computational regression model to predict whether a region a solid tumor biopsy is from the tumor or from the stroma, utilizing bulk spatial omics data to generate features. In many embodiments, the prediction of region type is determined by counting the number of expressed genes and assessing the abundance of nonimmune cells. In some embodiments, downstream analysis is performed upon predicting the region location.

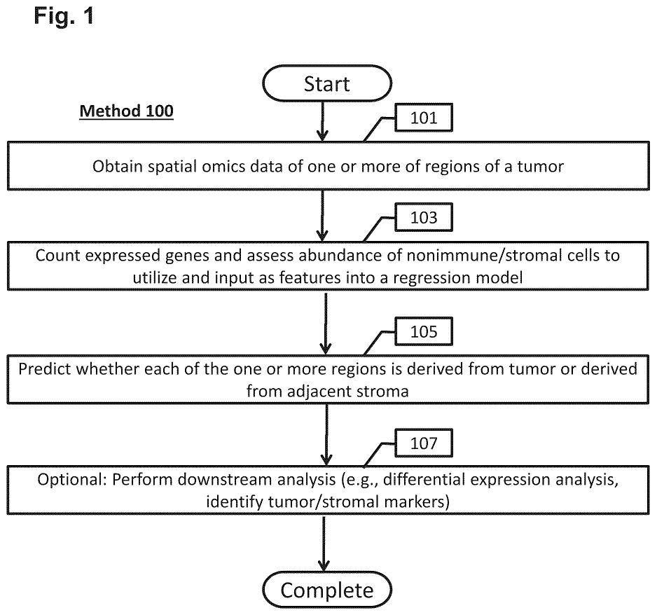

Provided in FIG. 1 is a computational method to annotate a bulk spatial transcriptomic sequencing result as tumor or stroma. Method 100 can begin by obtaining spatial omics data from a plurality of regions of a solid tumor specimens. A specimen is a collection of cells having a plurality of cell types that are defined by a spatial arrangement. Generally, a solid tumor specimen can be derived from a biopsy of a cancer patient. The omics data can be derived from a living specimen or from a fixed specimen, as appropriate to the methodology to perform spatial omics assessment.

To perform bulk spatial transcriptomics, spatial positions within of a specimen can be labeled, RNA can be extracted from the specimen, processed and assessed. Or alternatively, the processing and assessing of RNA can be performed in situ. Various methods for assessing the transcriptome can be utilized, including (but not limited to) in situ sequencing, microarrays, and RNA-sequencing. RNA-sequencing can be whole exome sequencing, capture targeted sequencing, amplification-based targeted sequencing, sequencing based on random priming, or end-biased sequencing, with or without unique molecular identifiers (UMIs). Generally, bulk spatial transcriptomic sequencing methods have low resolution (between about 5 and 20 cells per region) but can provide near-complete transcriptome depth. A number of platforms have been developed for performing spatial transcriptomics. Examples for RNA-seq transcriptomics include (but are not limited to) 10× Genomics Visium and NanoString GeoMX, each of which can be combined with high-throughput sequencers (e.g., Illumina HT series) (for more on Visium, see P. L. Stahl, et al., Science. 2016 Jul. 1; 353(6294):78-82; for more on GeoMX, see K. Roberts, bioRxiv 2021.03.20.436265; the disclosures of which are each incorporated herein by reference).

In many implementations, the source material for performing spatial omics for predicting tumor or stroma is captured in a plurality of regions, which may include tumor regions and adjacent stroma region. Generally, the plurality of regions covers the specimen to be assessed, or at least a portion thereof. Various methods can be utilized to capture source material from the plurality regions, which may be dependent on various protocols and particular type of omics to be assessed. In some implementations, the source material is extracted from the plurality of regions using laser capture microdissection. In some implementations, the source material is extracted from the plurality of regions using iterative microdigestion. In some implementations, the source material is extracted from the plurality of regions using in situ capture.

Upon sequencing a region, the spatial omics data can be retrieved. The spatial omics data can be further processed to ensure high data quality for further downstream assessment. For example, reads that map poorly in a sequencing result can be discarded. Many other processing steps can be performed, as is routine when assessing omics data.

Method 100 counts (103) expressed genes and assesses abundance of nonimmune/stromal cells to utilize and input as features into a regression model. To count expressed number of genes, each unique gene within the sequencing result of a region is counted. Abundances of non/non-stromal cells can be inferred by deconvolution methods, such as spacexr (D. M. Cable, et al., Nat Biotechnol. 2022 April; 40(4):517-526, the disclosure of which is hereby incorporated by reference). In some implementations, counts of expressed genes is further performed in one-hop neighboring stops. In some implementations, abundances of non/non-stromal cells can be inferred is further performed in one-hop neighboring stops.

Method 100 predicts (105) whether each of the one or more regions is derived from tumor or from adjacent stroma. To do so, a trained regression model (e.g., logistic regression) is trained. In some implementations, the regression model is trained with supervision, in which features are labeled as tumor derived or stroma derived. It was found that counts of expressed genes and abundance of nonimmune/stromal cells are good features for predicting whether a region of bulk spatial transcriptomic data is derived from tumor. In some implementations, features for the regression model include counts of expressed genes and abundance of nonimmune/stromal cells. In some implementations, features for the regression model include counts of expressed genes, abundance of nonimmune/stromal cells, counts of gene cells in one-hop neighboring spots, and abundance of nonimmune/stromal in one-hop neighboring spots.

Upon counting the expressed genes and assessing the abundance of nonimmune/stromal cells, these data are used as features to predict whether the bulk spatial transcriptomic data is from the tumor or the adjacent stroma.

Method 100 optionally performs (107) downstream analysis. The downstream analysis can include (but is not limited to) differential analysis or identification of markers of tumor or stromal.

Differential analysis can be performed to identify phenotypic differences between tumor and stroma. These phenotypic differences can also be used to methodically determine markers specific to tumor tissue and specific to stromal tissue. The analysis can be done at a single cell resolution to compare the differences of a cell type when it is within the tumor against when it is within the stroma. To perform differential analysis, single-cell omic profiles of tumors and stroma can be generated. While differential analysis typically focuses on transcriptomic differences (e.g., differential gene expression analysis), it can be performed on any omic phenotype. For methylomics, differences of CpG methylation can be assessed. For epigenomics, differences of chromatin condensation or chromatin markers can be assessed. For proteomics, differences in protein levels, protein modification, and the like can be assessed. For differential analysis on any omics, the phenotypes with the greatest differences can be determined. For example, methylation of CpG sites with the highest log fold change within the tumor compared to stroma of can be markers for identifying whether a particular immune cell (e.g., macrophage) is within the tumor. The assessment can be performed using on a particular cell type, on an aggregate of cells of all cell types, on an aggregate of a certain set of cell types, or an aggregate across cancer types. In some implementations, a top number of phenotype differences are utilized as markers (or as a signature), which can be the top 5, the top 10, the top 20, the top 50, the top 100, the top 200, etc., phenotype differences. These markers can be useful, for instance, when performing single-cell methyl sequencing, and determining whether a particular single-cell methyl-seq result is within the tumor or stroma.

While specific examples of methods for predicting whether a region is tumoral or stromal using bulk spatial omics is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

A spatial ecotype is a regional neighborhood of cells that are composed of a set of particular cell types, where each cell of cell type within the spatial ecotype shares an omic phenotype. Spatial ecotypes are useful for understanding regionalized dynamics within complex tissue arrangements. Cells within the local regions within a tissue are influenced by regionalized dynamics. For example, when a certain tissue region is undergoing inflammation, the composition of cells within that region is influenced by the cell type of the tissue, infiltrating immune cells, and the division/maturation of cells to repair the region. These regionalized dynamics are especially present within solid tumors, which are composed of a complex array of many cell types that are influenced by intrinsic factors such as de novo mutagenesis, neoplasticity, cellular division, epigentic modifications, and chromosomal rearrangements; and by extrinsic factors such as nutrient resources, inflammation, immune cell activation, cell-cell contact, and local signaling factors. While tumors and cancer types are often considered heterogenous and disorganized, there is benefit in learning the conservation of regionalized dynamics to better diagnose patients, better inform treatment responsiveness, and further develop better therapeutics.

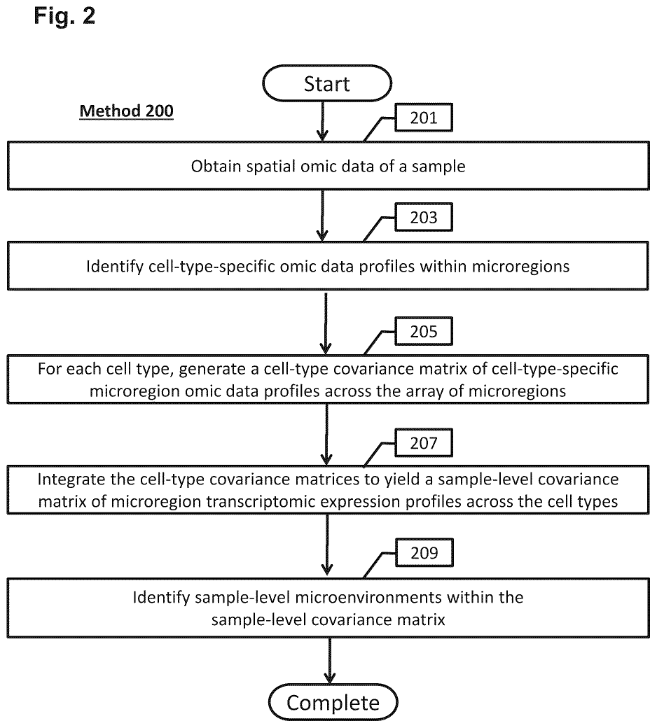

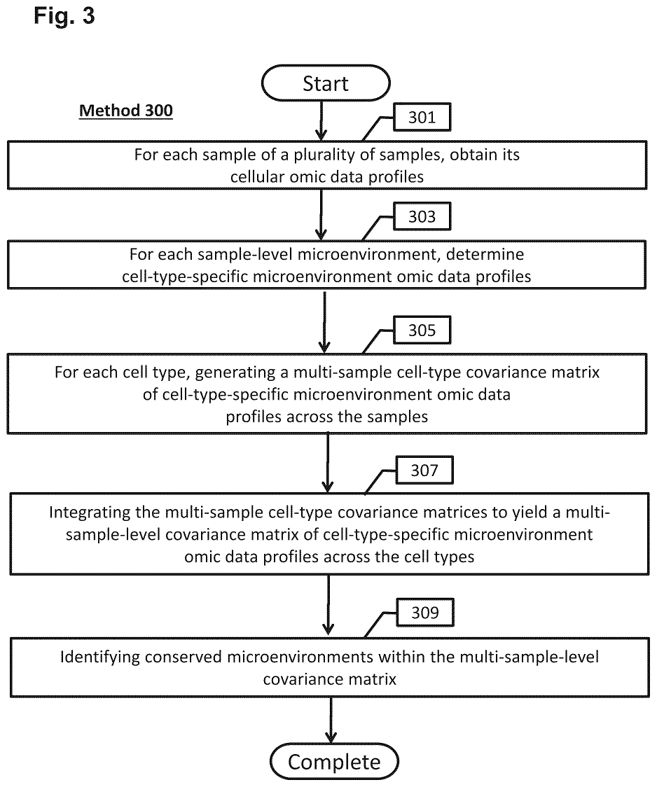

Provided in FIGS. 2 and 3 are computational frameworks that enable identification of microregions within a tissue. Further, by examining covariance among these microregions, recurrent microregions within a sample are identified, which can be referred to as microenvironments (FIG. 2). By further assessing covariance of microvariants across samples, conserved microenvironments across the samples can be identified (FIG. 3). Conserved microenvironments are also referred to as spatial ecotypes, as these regions recurrently are identified with a type of tissue and comprise a spatial identity and a composition of cell types having shared cell state. The methods of FIGS. 2 and 3 extensively analyze omic data at multiple biological levels, and computing covariance of the data. Such analysis is useful in analyzing any multicellular network, but especially tissues. The methods have an ability to interpret local crosstalk between tissue cells and the immune cells that pass through and infiltrate the solid tissue. Accordingly, the methods are useful for assessing fundamental biology, disease pathology, and medical intervention.

Provided in FIG. 2 is a computational method to identify microenvironments within a tissue sample, utilizing spatial omic data. Method 200 can begin by obtaining (201) spatial omics data from a sample. A sample is specimen that is a collection of cells having a network of cell types that interact and communicate. In some implementations, a specimen is derived from an in vivo source, such as a tissue specimen from a patient or other animal. In some implementations, a specimen is derived from an in vitro source, such as a multicellular organoid or other multicellular system. A spatially defined can be a primary tissue specimen, a culture of mixed cells, an organoid, or any other specimen that is a multicellular network. In various examples, the specimen is a tumor, a multicellular organ specimen, a multicellular organoid specimen, a specimen comprising tissue infiltrated by immune cells, a specimen comprising host tissue and pathogens, or a specimen comprising host tissue and microbiomes. The omics data can be derived from a living specimen or from a fixed specimen, as appropriate to the methodology to perform spatial omics assessment.

Any spatial omics data can be utilized provided it can differentiate the cellular phenotypes within the specimen. Spatial omics that can be assessed include (but are not limited to) is spatial transcriptomics, spatial epigenomics, spatial methylomics, or spatial proteomics. As dependent on the omics type, biomolecules can be collected from the specimen and processed for perform the omics analysis.

To perform spatial transcriptomics, RNA can be extracted from the specimen, processed and assessed. Any appropriate method for assessing the transcriptome can be utilized, including (but not limited to) in situ hybridization, in situ sequencing, microarrays, and RNA-sequencing. RNA-sequencing can be whole exome sequencing, capture targeted sequencing, amplification-based targeted sequencing, sequencing based on random priming, or end-biased sequencing, with or without unique molecular identifiers (UMIs). When deciding on how to assess the transcriptome, there is a balance between the depth of genes analyzed and spatial resolution. For instance, in situ methods have subcellular resolution but cannot assess a large depth of genes whereas sequencing methods have lower resolution but can provide near-complete transcriptome depth. Further, various methodologies can be utilized to impute single-cell transcriptomic data onto spatial transcriptomic data (M. R. Vahid, et al., Nat Biotechnol. 2023 November; 41(11):1543-1548, the disclosure of which is hereby incorporated by reference). A number of platforms have been developed for performing spatial transcriptomics. Examples for situ hybridization transcriptomics include (but are not limited to) Vizgen MERSCOPE, NanoString CosMX, 10× Genomics Xenium, and hybridization-based in situ sequencing (HybISS) (for more on MERSCOPE, see J. Liu, et al., Life Sci Alliance. 2022 Dec. 16; 6(1):e202201701; for more on CosMX, see S. He, et al., Nat Biotechnol. 2022 December; 40(12):1794-1806; for more on Xenium, see S. M. Salas, et al., bioRxiv 2023.02.13.528102; for more on HybISS, see D. Gyllborg, et al., Nucleic Acids Res. 2020 Nov. 4; 48(19):e112; the disclosure of which are each incorporated herein by reference). Examples for RNA-seq transcriptomics include (but are not limited to) 10× Genomics Visium and NanoString GeoMX, each of which can be combined with high-throughput sequencers (e.g., Illumina HT series) (for more on Visium, see P. L. Stahl, et al., Science. 2016 Jul. 1; 353(6294):78-82; for more on GeoMX, see K. Roberts, bioRxiv 2021.03.20.436265; the disclosures of which are each incorporated herein by reference).

To perform spatial epigenomics, DNA or RNA can be extracted from the specimen, processed and assessed. Any appropriate method for assessing the epigenome can be utilized, including (but not limited to) chromatin-immunoprecipitation sequencing, chromatin access assessment, and as inferred from RNA-sequencing or DNA methylation-sequencing. Chromatin access assessment can be performed using (for example) assay for transposase-accessible chromatin with sequencing (ATAC-Seq). Further, epigenomic data can be imputed onto spatial transcriptomic data, by adapting methods previously performed for imputing single-cell transcriptomic data (M. R. Vahid, et al., Nat Biotechnol. 2023 November; 41(11):1543-1548, the disclosure of which is hereby incorporated by reference).

To perform spatial methylomics, DNA or RNA can be extracted from the specimen, processed and assessed. Any appropriate method for assessing the methylome can be utilized, including (but not limited to) methylation assessment and as inferred from RNA-sequencing. Methylation assessment can be performed using (for example) bisulfite conversion sequencing or enzymatic methyl sequencing (EM-Seq). Further, single-cell methylomic data can be imputed onto spatial transcriptomic data, by adapting methods previously performed for imputing single-cell transcriptomic data (M. R. Vahid, et al., Nat Biotechnol. 2023 November; 41(11):1543-1548, the disclosure of which is hereby incorporated by reference).

To perform spatial proteomics, proteinaceous species can be extracted from the specimen, processed and assessed. Any appropriate method for assessing the proteome can be utilized, including (but not limited to) mass spectrometry and protein microarrays.

In many implementations, the source material for performing spatial omics is captured in an array of regions, which can generally span a section of a specimen to be assessed. Various methods can be utilized to capture source material from the plurality regions, which may be dependent on various protocols and particular type of omics to be assessed. In some implementations, the source material is extracted from the plurality of regions using laser capture microdissection. In some implementations, the source material is extracted from the plurality of regions using iterative microdigestion. In some implementations, the source material is extracted from the plurality of regions using in situ capture.

In many implementations, spatial omics is performed in situ, meaning the omics analysis is performed directly on an intact specimen. Generally, a fixed specimen (e.g., formalin fixed paraffin embedded tissue) or a fresh frozen specimen is permeabilized and detection of biomolecules for omics analysis is performed therein. Because in situ omics is performed directly on the specimen and provides subcellular resolution, a plurality regions can be defined as desired by the user and can be as granular as a single cell.

Upon assessment of a plurality of regions, spatial omics data can be retrieved. The spatial omics data can be further processed to ensure high data quality for further downstream assessment. For example, reads that map poorly in a sequencing result can be discarded. Many other processing steps can be performed, as is routine when assessing omics data.

Method 200 identifies (203) cell-type-specific omic data profiles within microregions. A microregion can generally be defined by an area, such a radial distance. The microregion size can be of a variety of sizes, but should remain constant for analysis of a sample (and across samples when performing multi-sample analysis). For each microregion, the framework can encode spatial omic data into cell-type-specific microregion omic data profiles. For each cell type within a microregion, omic data profiles can be aggregated, such as averaging the omic data (e.g., averaging of gene expression level or CpG methylation level). In some implementations, omic data profiles are converted into vectors.

For each cell type, Method 200 generates (205) a cell-type covariance matrix of cell-type-specific microregion omic data profiles across the array of microregions. A matrix E^{c} of dimension g genes by m microregions can be generated. To focus on multicellular networks, in some implementations, microregions characterized by a sing cell type can be removed. Additionally, other pruning of microregions can be performed to enhance analysis. For example, in some implementations, genes that are uncommonly expressed within the microregions are removed. In some implementations, if a microregion does not have the cell type for which the covariance matrix is being generated, those microregions are removed. In some implementations, microregions expressing a low number of genes are removed.

For each cell type, covariance networks can be constructed to identify shared microregions. Optionally, each cell-type covariance matrix can be dimensionally reduced to better handle the matrices. Any appropriate dimension reduction technique can be utilized, such as (for example) principal component analysis, linear discriminant analysis, and T-distributed Neighbor Embedding. Pairwise covariance can be computed between the cell-type-specific microregion omic data profiles.

It is likely that a high number of covariances would be found within a sample. In some implementations, only the top covariances are kept. The number of top covariances can vary, and is robust to range of values. In various implementations, the number of top covariances kept is five or less, 10 or less, 20 or less, 50 or less, 100 or less, 200 or less, or 500 or less.

Method 200 integrates (207) the cell-type covariance matrices to yield a sample-level covariance matrix of microregion omic data profiles across the cell types. Various methods can be utilized for integration. In some implementations, a sample-level covariance matrix is formed by fusing cell-type covariance matrices via a similarity network fusion method. The sample-level covariance matrix should result in a matrix that encodes spatial community structure by linking microregions exhibiting high covariation across cell types.

Method 200 identifies (209) sample-level microenvironments within the sample-level covariance matrix. Generally, a grouping technique can be utilized to identify microenvironments within the sample. In some implementations, a clustering technique is utilized, such as a Seurat clustering method. The resulting grouping yields microenvironments of the sample that are distributed along the sample in spatial context. Each microenvironment shares a composition of cell types, the cells of the cell type generally sharing an omic phenotype.

While specific examples of methods for identifying microenvironments within a sample using spatial omics data are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

Provided in FIG. 3 is a computational method to identify conserved microenvironments across samples. The samples can be of the same specimen, can be derived from a single individual, can be derived from multiple individual, and/or can be of multiple tissue types. For example, identifying pan-cancer conserved microenvironments can be achieved by assessing samples of several tissue types and cancer types. Method 300 can begin by, for each sample of a plurality of samples, obtaining (301) cellular omic data profiles of the microregions that utilized to identify the microenvironments. The omic data profiles can be generated in a manner in accordance with FIG. 2, or any appropriate method that yields sample-level microenvironments utilizing spatial omics.

The framework can compute cell-type-specific omic data profiles for each microenvironment, inclusive of all samples to be utilized for identifying conservation. To do so, each cell can be assigned to a sample-level microenvironment, which can be based on the spatially nearest microregion that the cell resides within. For each cell type, cell omic data profiles assigned to the same sample-level microenvironment can be aggregated such as averaging the omic data. In some implementations, omic data profiles are converted into vectors.

For each sample-level microenvironment, Method 300 determines (303) cell-type-specific microenvironment omic data profiles. The framework can compute cell-type-specific omic data profiles for each microenvironment, inclusive of all samples to be utilized for identifying conservation. To do so, each cell can be assigned to a sample-level microenvironment, which can be based on the spatially nearest microregion that the cell resides within. For each cell type, cell omic data profiles assigned to the same sample-level microenvironment can be aggregated such as averaging the omic data. In some implementations, microenvironment omic data profiles are converted into vectors.

For each cell type, Method 300 generates (305) a multi-sample cell-type covariance matrix of cell-type-specific microenvironment omic data profiles across the samples. A matrix E′^{c}, each of dimension g genes by s sample-level spatial clusters can be generated. In some implementations, genes to that are utilized have nonzero expression within at least one microregion of each sample utilized. Additionally, other pruning of microregions can be performed to enhance analysis. For example, in some implementations, genes that are uncommonly expressed within the microenvironments are removed. In some implementations, if a microenvironment does not have the cell type for which the covariance matrix is being generated, those microenvironments are removed. In some implementations, microenvironments expressing a low number of genes are removed. In some implementations, the features of the vector were reduced to only keep top most variable genes in the cell type. In various implementations, the number of top most v kept is 20 or less, 50 or less, 100 or less, 150 or less, 200 or less, 250 or less, or 500 or less.

For each cell type, multi-sample covariance networks can be constructed to identify shared microenvironments. Optionally, each multi-sample cell-type covariance matrix can be dimensionally reduced to better handle the matrices. Any appropriate dimension reduction technique can be utilized, such as (for example) principal component analysis, linear discriminant analysis, and T-distributed Neighbor Embedding. Spearman correlation and pairwise covariance can be computed between the cell-type-specific microregion omic data profiles.

Method 300 integrates (307) the multi-sample cell-type covariance matrices to yield a multi-sample-level covariance matrix of cell-type-specific microenvironment omic data profiles across the cell types. Various methods can be utilized for integration. In some implementations, a multi-sample-level covariance matrix is formed by fusing cell-type covariance matrices via a similarity network fusion method. The multi-sample-level covariance matrix should result in a matrix that encodes spatial community structure by linking microenvironments exhibiting high covariation across cell types.

Method 300 identifies (309) conserved microenvironments within the multi-sample-level covariance matrix. Generally, a grouping technique can be utilized to identify conserved microenvironments across the samples. In some implementations, a clustering technique is utilized, such as a non-negative matrix factorization method. The resulting grouping yields conserved microenvironments of the sample that are shared among the samples. The conserved microenvironments are also referred to as spatial ecotypes.

An omic data profile for each conserved microenvironment can be generated, which can help understand the cell state of each cell type within the conserved microenvironment. Generally, for each cell type, omic data of a number of cells within a conserved microenvironment can be combined clustering technique, such as a non-negative matrix factorization method. To keep ensure balanced representation, the same number of cells from each sample-level microenvironment can be utilized to aggregate the omic data of the selected cells. To facilitate cell state recovery of a cell type of a conserved microenvironment, a number uniquely highly present omic data can be selected. For example, for gene expression within a cell type, a top number of genes (e.g., top 10, 20, 50, or 100 genes) can be chosen per conserved microenvironments based on the largest positive delta compared to the second highest expression across the rest of the conserved microenvironments. Each basis matrix was then reduced to the selected genes, which can be utilized to recover conserved-microenvironment-specific cell states from an external single-cell omic dataset.

In some implementations, cell states specific to a conserved microenvironment are determined. To identify and validate cell states specifically enriched in each conserved microenvironment, identification of conserved microenvironment cell state omic data profiles can be repeated on subsets of the data utilized for discovery. The resulting basis matrices can be used to predict cell state labels on held out data in a leave-one-sample-out cross-validation (LOOCV) process. The enrichment of each cell state within each conserved microenvironment can be assessed by its ability to correctly assign cells to that conserved microenvironment. The identified conserved-microenvironment-specific cell states can be utilized to generate a basis matrix. A can be trained with conserved-microenvironment-specific cell states non-negative matrix factorization by repeating the process of generating a basis matrix to yield an ensemble of basis matrices for use to recover conserved microenvironments.

While specific examples of methods for identifying conserved microenvironments across samples are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

In addition to being able to recover conserved-microenvironment-specific cell states from an external single-cell omic dataset, it is desirable to recover conserved-microenvironment-specific cell states from bulk sequencing as well. Provie in FIG. 4 is a computational method incorporating a deconvolution to recover conserved-microenvironment-specific cell states from bulk sequencing. Method 400 begins by identifying (401) conserved microenvironments within a plurality of tissue samples. Conserved microenvironments can be identified as described in FIG. 3, or any other appropriate method.

Method 400 assembles (403) pseudo-bulk mixtures from single-cell RNA data. Each cell of the scRNA data is assigned to a conserved microenvironment as identified in step 401. If a cell does not match the cell state of a conserved microenvironment, it is assigned to nonconserved. If multiple tissue types are utilized, in some implementations pseudo-bulk mixtures that are generated are confined to only that tissue type. The number of pseudo-bulk mixtures to be generated for each tissue type can vary, but generally higher numbers of pseudo-bulk mixtures can provide better derivation of matrices. In various implementations, at least 10 psuedo-bulk mixtures per tissue type, at least 20 psuedo-bulk mixtures per tissue type, at least 50 psuedo-bulk mixtures per tissue type, at least 100 psuedo-bulk mixtures per tissue type, at least 150 psuedo-bulk mixtures per tissue type, at least 200 psuedo-bulk mixtures per tissue type, or at least 500 psuedo-bulk mixtures per tissue type are generated. A gene expression profile can be constructed for each pseudo-bulk mixture, aggregating a number of cells randomly selected but also satisfying the predefined fractions of cell states for a cell type. The number of cells can vary, but should be generally representative of bulk sequencing. In various embodiments, at least 100 cells, at least 200 cells, at least 500 cells, at least 1000 cells, at least 2000 cells, at least 5000 cells are randomly selected.

Method 400 derives (405) a basis matrix by non-negative matrix factorization. The gene expression profiles of the pseudo-bulk mixtures can be combined into a gene by sample matrix. In some implementations, only genes observed in all samples are utilized. The fractional abundances of conserved microenvironments can be encoded into sample by label matrix. These two matrices can be utilized to derive a basis matrix by application of non-negative matrix factorization followed by feature selection. In some implementations, a top number of genes (e.g., top 10, 20, 50, or 100 genes) are chosen per conserved microenvironment based on the largest positive delta compared to the second highest expression across the other conserved microenvironment.

In some implementations, cell states specific to a conserved microenvironment are determined. To identify and validate cell states specifically enriched in each conserved microenvironment, identification of conserved microenvironment cell state omic data profiles can be repeated on subsets of the data utilized for discovery. The resulting basis matrices can be used to predict cell state labels on held out data in a leave-one-sample-out cross-validation (LOOCV) process. The enrichment of each cell state within each conserved microenvironment can be assessed by its ability to correctly assign cells to that conserved microenvironment. The identified conserved-microenvironment-specific cell states can be utilized to generate a basis matrix. A can be trained with conserved-microenvironment-specific cell states non-negative matrix factorization by repeating the process of generating a basis matrix to yield an ensemble of basis matrices for use to recover conserved microenvironments.

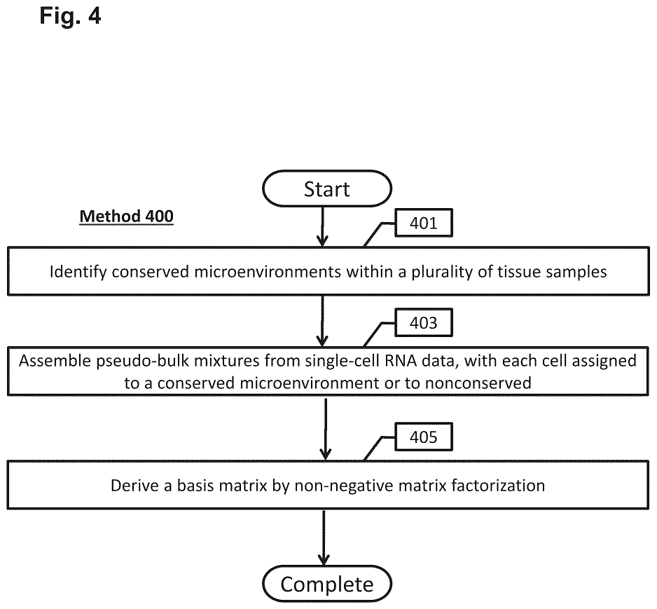

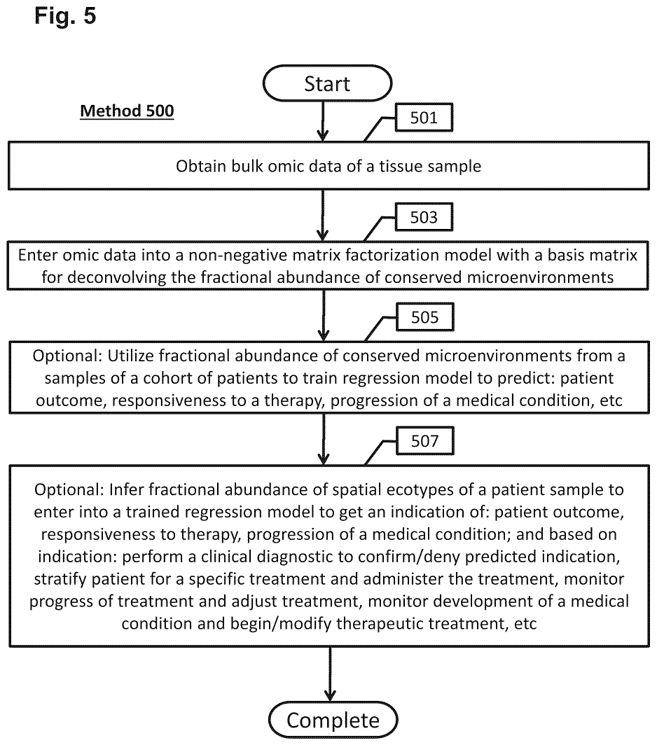

Upon development of the basis matrices for recovery of conserved microenvironment within bulk sequencing, these matrices can be used in a clinical setting. Provided in FIG. 5 is an example of a method to assess bulk sequencing data of a solid tissue to identify the fractional abundance of conserved microenvironments in the tissue sample. Method 500 can begin by obtaining (501) bulk omic data of a tissue sample. Generally, any deep sequencing technique can be utilized to generate the omic data, such as RNA-seq, methyl-seq, ATAC-seq, etc.

The tissue sample can be derived from a patient to be diagnosed. As noted in the Examples described herein, many medical conditions can be assessed, including (for example) cancers, autoimmune disorders, autoinflammatory disorders, transplant rejection, liver disease, renal disorders, and pathogenic infection. Generally, any medical condition in which tissue is damaged or results in immune cell infiltration can be assessed. For each medical condition, conserved microenvironments can be identified, and these conserved microenvironments can be indicative of (for example) patient outcome, responsiveness to a therapy, or progression of medical condition.

Method 500 enters (503) the bulk omic data in a non-negative matrix factorization model with a basis matrix for deconvolving the fractional abundance of conserved microenvironments. The non-negative matrix factorization model with a basis matrix can be generated using conserved-microenvironment-specific cell states, such that the relative abundance of each conserved microenvironment can be inferred.

Method 500 optionally utilizes (505) fractional abundance of conserved microenvironments from samples of a cohort of patients to train regression model to predict a medical phenotype, such as (for example) patient outcome, treatment response, development of a medical condition. The computational model can be a machine-learning model capable of learning an association. To do so, a cohort patients with a medical phenotype can have fractional abundance of conserved microenvironments determined from omic data derived from an affected tissue source. The fractional abundances of can be labeled with the medical phenotype and entered into a machine-learning regression model to learn an association between fractional abundances and the clinical phenotype. Upon training, the model can be utilized for diagnostics.

Method 500 optionally (507) infers fractional abundance of spatial ecotypes of a patient sample to enter into a trained regression model to get an indication of a medical phenotype such as patient outcome, responsiveness to therapy, or progression of a medical condition. Based on the indication of the medical phenotype, several clinical actions can be taken, such as perform a clinical diagnostic to confirm or deny the predicted indication, stratify the patient for a specific treatment and administer the treatment, monitor progress of a treatment and adjust treatment when indicated, monitor development of a medical condition, and begin or modify therapeutic treatment.

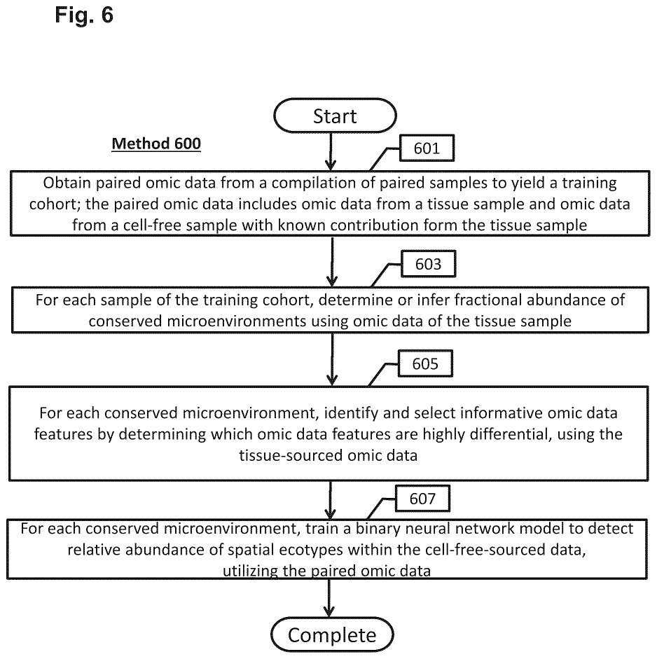

Conserved environments can also be inferred from omic data generated from a cell-free source. It was determined that a binary neural network can be trained to differentiate whether omic data from a cell-free source is includes biomolecules from conserved environments and the relative abundance of the conserved environments. FIG. 6 provides a method for training a binary neural network to infer relative abundance of conserved environments.

Method 600 obtains (601) paired omic data from a compilation of paired samples to yield a training cohort. For example, the paired samples can be methylation data from a cell-free source and RNA-seq data from solid tissue. The contribution within the cell-free sample from the tissue sample is known or can be determined.

Various cell-free sources may be utilized as appropriate to the tissues being assessed. Generally, blood or plasma is useful for assessment of all tissues, as it is common for cells of injured or inflamed tissue to release biomolecules into the blood circulatory system. Other cell-free sources that are useful include urine for assessment of urethral or bladder tissue, stool for assessment of colorectal tissue, and saliva for assessment of inner mouth tissues.

For each sample of the training cohort, Method 600 determines (603) or infers fractional abundances of the conserved microregions using omic data of the solid tissue sample. For example, bulk omic sequencing data can infer fractional abundances of the conserved microregions via a non-negative matrix factorization model, such as the example described within FIG. 5. Single-cell omic data can infer fractional abundances based on its cell state, and assigning cells to a conserved microregion.

For each conserved microenvironment, Method 600 identifies (605) and selects informative omic data features by determining which omic data features are highly differential, using the solid tissue-sourced omic data. Samples with an inferred fraction that are above an upper threshold (e.g., 66%, 75%, 80%, 90%) are put in a first group and samples with an inferred fraction that are below a lower threshold (e.g., 33%, 25%, 20%, 10%) are put into a second group. The two threshold can be selected empirically or otherwise, but should be far enough apart to differentiate cell states between the two groups. The cell-free omic data is put into the first group or into the second group based on its paired solid tissue grouping. Differential analysis is performed on the cell-free derived omic data, comparing the first group and the second group. Features of the cell-free omic data are selected based on the differential analysis results, in which highly differential features are selected.

For each conserved microenvironment, Method 600 trains (607) a binary neural network to detect relative abundance of conserved microenvironments utilizing mixtures of cell-free omic data with associated conserved microenvironment abundance and other omic data unassociated with any conserved microenvironment. The mixtures have known ratios, enabling training of the model to infer relative abundance of conserved microenvironments in cell-free samples cell-free sources that have a large amount of background molecules.

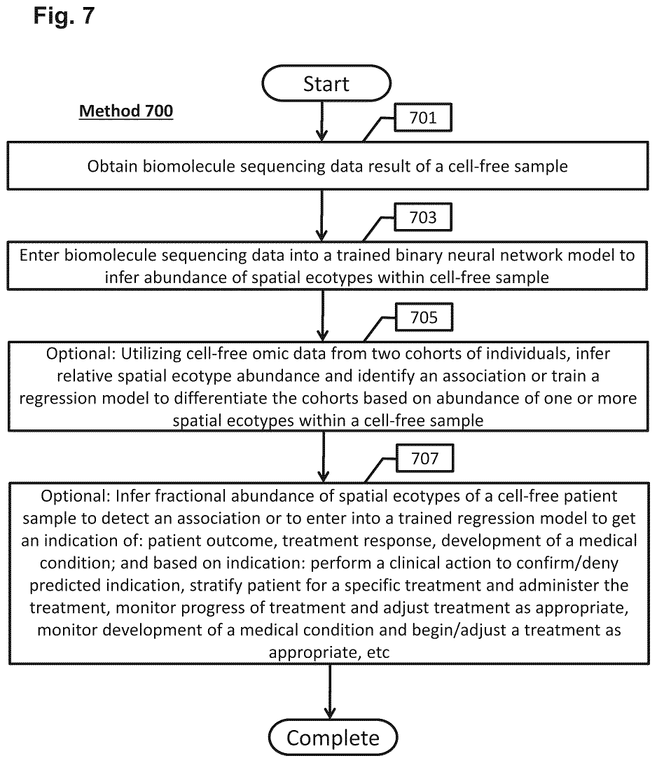

Upon development of the binary neural network for inferring relative abundance of conserved microenvironments from a cell-free source, the predictive model can be used in a clinical setting. Provided in FIG. 7 is an example of a method to assess sequencing data of a sample from a cell-free source to identify the fractional abundance of conserved microenvironments in the tissue of interest. Method 700 can begin by obtaining (701) biomolecule sequencing data result of a cell-free sample. Generally, any deep sequencing technique can be utilized to generate the sequencing data, such as RNA-seq, methyl-seq, ATAC-seq, etc.

The cell-free sample can be derived from a patient to be diagnosed. As noted in the Examples described herein, many medical conditions can be assessed, including (for example) cancers, autoimmune disorders, autoinflammatory disorders, transplant rejection, liver disease, renal disorders, and pathogenic infection. Generally, any medical condition in which tissue is damaged or results in immune cell infiltration can be assessed. For each medical condition, conserved microenvironments can be identified, and these conserved microenvironments can be indicative of (for example) patient outcome, responsiveness to a therapy, or progression of medical condition.

Method 700 enters (703) biomolecule sequencing data into a trained binary neural network model to infer abundance of spatial ecotypes within cell-free sample. The binary model can be trained to predict abundance of spatial ecotypes in cell-free samples that include background data, such as the training method described in FIG. 6.

Method 700 optionally utilizes (705) fractional abundance of conserved microenvironments from samples of a cohort of patients to train regression model to predict a medical phenotype, such as (for example) patient outcome, treatment response, development of a medical condition. The computational model can be a machine-learning model capable of learning an association. To do so, a cohort patients with a medical phenotype can have fractional abundance of conserved microenvironments determined from omic data derived from an affected tissue source. The fractional abundances of can be labeled with the medical phenotype and entered into a machine-learning regression model to learn an association between fractional abundances and the clinical phenotype. Upon training, the model can be utilized for diagnostics.

Method 700 optionally (707) infers fractional abundance of spatial ecotypes of a patient sample to enter into a trained regression model to get an indication of a medical phenotype such as patient outcome, responsiveness to therapy, or progression of a medical condition. Based on the indication of the medical phenotype, several clinical actions can be taken, such as perform a clinical diagnostic to confirm or deny the predicted indication, stratify the patient for a specific treatment and administer the treatment, monitor progress of a treatment and adjust treatment when indicated, monitor development of a medical condition, and begin or modify therapeutic treatment.

As noted, diagnostic procedures can be developed for a number of clinical phenotypes. Described in the example are some examples related to cancer. The examples show that the methods described herein apply to cancer generally, as the data was trained using pan-cancer sources. Nine spatial ecotypes were found to be conserved throughout cancer, which can be referred to as SE1, SE2, . . . , and SE9. Of major interest are the spatial ecotypes that inform the benefit of immune checkpoint inhibitors for the treatment of cancer. It was found that great relative abundance of SE8 and SE7 indicated benefit from immune checkpoint inhibitor therapy. Conversely, it was found that greater relative abundance SE4 indicated resistance to immune checkpoint inhibitor therapy. Accordingly, a diagnostic method for determining whether a cancer patient would benefit from administration immune checkpoint inhibitor can be performed using tumor tissue or a cell-free source as described herein. When the diagnostic method indicates a presence of SE8 and/or SE7, the patient is administered an immune checkpoint inhibitor. Examples of immune checkpoint inhibitors include nivolumab, pembrolizumab, dostarlimab, sintilimab cemiplimab, ipilimumab, tremelimumab, tezolizumab, durvalumab, avelumab. When the diagnostic method indicates a presence of SE4, a therapy alternative to an immune checkpoint are administered. Further, expression of STMN1, TUBB, TYMS or GZMB within CD8 T-cells; expression of TNFRSF4, TNFRSF18, IL2RA, CTLA4, or FOXP3 within CD4 T-cells; expression of CCL8 or ISG15 within macrophages; expression of CXCL8 or MMP1 within fibroblasts; and expression of CEACAM1 or CEBPB within endothelial cells are each associated with SE8 or SE7, and thus can be used as diagnostic markers for benefit of immune checkpoint inhibitor therapy.

Various diagnostic methods can be performed for a number of biomedical conditions. In some implementations, a screening method or a diagnostic comprises:
extract or collect a cell-free sample or solid tissue sample
sequence the cell-free sample or solid tissue sample to yield omic data
determine relative abundance of one or more spatial ecotypes
determine an indication of:
   patient outcome
   responsiveness to a therapy
   biomedical condition progression
   presence of minimal residual disease
   efficacy of a therapy
treat the patient based on the indication

In some implementations, a cell-free sample is (or is derived from) blood, plasma, lymph, cerebrospinal fluid, amniotic fluid, urine, or stool. In some implementations, the screening method is generalized and assesses a plurality of biomedical conditions. In some implementations, the diagnostic method is a particular biomedical condition. In some implementations, a biomedical condition is identified by relative abundance of one or more spatial ecotypes. In some implementations, a biomedical condition is identified by a computational model trained to predict the biomedical condition utilizing the relative abundances of one or more spatial ecotypes.

In some implementations, the biomedical condition is pregnancy. In some implementations, the biomedical condition is a fetal complication. In some implementations, the biomedical condition is a pregnancy complication. When assessing a fetal complication or a pregnancy complication, in some implementations, the cell-free sample is derived amniotic fluid. When assessing a fetal complication or a pregnancy complication, in some implementations, screening is performed at various timepoints throughout a pregnancy. In some implementations, when a fetal complication or a pregnancy complication is identified, further diagnostic procedures are performed, such as (for example) assessments for gestational diabetes, genetic assessment of the fetus, fetal ultrasound, and maternal blood testing. In some implementations, when a fetal complication or a pregnancy complication is identified, a treatment is performed such as (for example) inducing labor, administering a tocolytic medication, and performing a Caesarian delivery.

In some implementations, the biomedical condition is a pathogenic infection. In some implementations, the biomedical condition is immunological status, such as (for example) a vaccination status or prior pathogenic infection. In some implementations, when assessing a pathogenic infection, the cell-free sample is also enriched for pathogen sequences. In some implementations, when a pathogenic infection is identified, a treatment is performed (such as) administering an antipathogenic medication (e.g., antibiotic agent, antiviral agent, antiparasitic agent, etc.). In some implementations, the screening method monitors the pathogenic infection, an antipathogenic treatment response, an immunological response, a health condition, or any combination thereof.

In some implementations, the biomedical condition is immune activation, such as (for example) activation in response to a pathogen, activation in response to an immunization, or activation of an autoimmune disorder. In some implementations, the biomedical condition is inflammation. In some implementations, when the biomedical condition is an autoimmune disorder or inflammation, a treatment is performed such as (for example) administering an immune suppressor, and administering an anti-inflammatory agent.

In some implementations, the biomedical condition is an organ transplant rejection. In some implementations, an organ transplant rejection is identified by abundance of spatial ecotypes. In some implementations, the screening method is performed periodically after the host receives the transplant. In some implementations, when organ transplant rejection is identified, further diagnostic procedures are performed such as (for example) tissue biopsy of the organ, and medical imaging of the organ. In some implementations, when organ transplant rejection is identified, a treatment is performed such as (for example) administering an increased dose of immunosuppressant agents and administering stronger immunosuppressant agents.

In some implementations, the biomedical condition is neurodegeneration. When assessing for neurodegeneration, in some implementations, the cell-free sample is (or is derived from) cerebrospinal fluid. In some implementations, neurodegeneration is identified by gene signatures related to abundance of spatial ecotypes. In some implementations, when neurodegeneration is identified, further diagnostic procedures are performed such as (for example) medical screening, assessments of motor activity or speech, and assessments of cognition. In some implementations, when neurodegeneration is identified, a treatment is performed such as (for example) medications for reducing neurodegenerative symptoms.

In some implementations, the biomedical condition is cancer. In some implementations, the screening method is performed as part of a cancer surveillance effort (e.g., before symptoms of cancer are present or are recognized). In some implementations, the screening method is performed during treatment to assess the treatment response. In some implementations, the screening method is performed after treatment to assess whether residual cancer (e.g., MRD) exists after a treatment, which can be performed periodically. In some implementations, the diagnostic method informs cancer subtype, cancer stage, and/or treatment strategy.

The screening can be performed for a number of neoplasm types, including (but not limited to) acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, upper tract urothelial cancer, vaginal cancer, and vascular tumors. In some implementations, when the cancer to be assessed is colorectal or gastric cancer, the cell-free sample is (or is derived from) a stool sample. In some implementations, when the cancer to be assessed is bladder, kidney, prostate, or upper tract urothelial cancer, the cfRNA sample is (or is derived from) a urine sample.

In some implementations, when cancer is indicated, a number of follow-up clinical evaluations can be performed, including (but not limited to) physical exam, medical imaging, mammography, endoscopy, stool sampling, pap test, alpha-fetoprotein blood test, CA-125 test, prostate-specific antigen (PSA) test, biopsy extraction, bone marrow aspiration, and tumor marker detection tests. Medical imaging includes (but is not limited to) X-ray, magnetic resonance imaging (MRI), computed tomography (CT), ultrasound, and positron emission tomography (PET). Endoscopy includes (but is not limited to) bronchoscopy, colonoscopy, colposcopy, cystoscopy, esophagoscopy, gastroscopy, laparoscopy, neuroendoscopy, proctoscopy, and sigmoidoscopy.

In some implementations, when cancer is indicated, a number of treatments can be performed, including (but not limited to) surgery, chemotherapy, radiation therapy, immunotherapy, targeted therapy, hormone therapy, stem cell transplant, and blood transfusion. In some implementations, an anti-cancer and/or chemotherapeutic agent is administered, including (but not limited to) alkylating agents, platinum agents, taxanes, vinca agents, anti-estrogen drugs, aromatase inhibitors, ovarian suppression agents, endocrine/hormonal agents, bisphophonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, zoledronate, tykerb, daunorubicin, doxorubicin, epirubicin, idarubicin, valrubicin mitoxantrone, bevacizumab, cetuximab, ipilimumab, ado-trastuzumab emtansine, afatinib, aldesleukin, alectinib, alemtuzumab, atezolizumab, avelumab, axtinib, belimumab, belinostat, bevacizumab, blinatumomab, bortezomib, bosutinib, brentuximab vedoitn, briatinib, cabozantinib, canakinumab, carfilzomib, certinib, cetuximab, cobimetnib, crizotinib, dabrafenib, daratumumab, dasatinib, denosumab, dinutuximab, durvalumab, elotuzumab, enasidenib, erlotinib, everolimus, gefitinib, ibritumomab tiuxetan, ibrutnib, idelalisib, imatinib, ipilimumab, ixazomib, lapatinib, lenvatinib, midostaurin, nectiumumab, neratinib, nilotinib, niraparib, nivolumab, obinutuzumab, ofatumumab, olaparib, loaratumab, osimertinib, palbocicilib, panitumumab, panobinostat, pembrolizumab, pertuzumab, ponatinib, ramucirumab, reorafenib, ribociclib, rituximab, romidepsin, rucaparib, ruxolitinib, siltuximab, sipuleucel-T, sonidebib, sorafenib, temsirolimus, tocilizumab, tofacitinib, tositumomab, trametinib, trastuzumab, vandetanib, vemurafenib, venetoclax, vismodegib, vorinostat, and ziv-aflibercept. An individual may be treated, by a single medication or a combination of medications described herein. A common treatment combination is cyclophosphamide, methotrexate, and 5-fluorouracil (CMF).

### Systems of Spatial Ecotyping

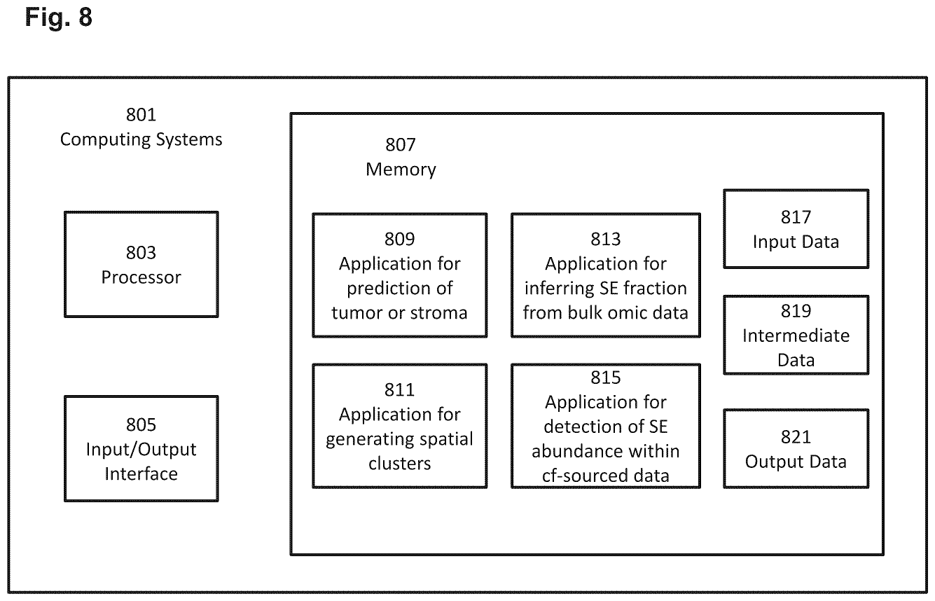

Turning now to FIG. 8, a computational processing system for cellular spatial alignment in accordance with various embodiments of the disclosure typically utilizes a processing system including one or more of a CPU, GPU and/or neural processing engine. In a number of embodiments, spatial omics input data is processed to spatially align cells using single cell omics data via a computational processing system. In some embodiments, the computational processing system is housed within a computing device that is in direct association a system for capturing spatial omics data or other sequencing data. In some embodiments, the computational processing system is housed separately from and receives the acquired spatial-omics data or other sequencing data. In certain embodiments, the computational processing system is implemented as a software application on a computing device such as (but not limited to) remote processor, CPU, mobile phone, a tablet computer, and/or portable computer.

A computational processing system in accordance with various embodiments of the disclosure is illustrated in FIG. 8. The computational processing system 801 includes a processor system 803, an I/O interface 805, and a memory system 87. As can readily be appreciated, the processor system 803, I/O interface 805, and memory system 807 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash). In the illustrated embodiment, the memory system is capable of storing a number of applications and/or data. Applications can include (but is not limited to) an application for prediction of tumor or stroma 809, an application for generating spatial clusters 811, an application for inferring spatial ecotype fraction from bulk omic data 813, and an application for detection of spatial ecotype abundance within cf-sourced data. The various applications can be downloaded and/or stored in non-volatile memory. When executed, the various applications are each capable of configuring the processing system to implement computational processes including (but not limited to) the computational methods described above and/or combinations and/or modified versions of the computational methods described above. In several embodiments, the various applications utilize input data 817, generate and/or utilize intermediate data 819, and generate output data 821, each of which can be stored in the memory system, which can be stored transiently for performing the computational methods or for longer terms such that the data can be retrieved at a later time point. Input data can include (but are not limited to) spatial-omics data, single cell sequencing data, bulk sequencing data, cell-free sequencing data. Intermediate data can include (but are not limited to) relative abundance of one or more spatial ecotypes. Output data ca include (but is not limited to) a diagnostic indication. It is to be understood that input data 817, intermediate data 819, and output data 821 can be utilized in number of different ways and thus should not be limited in any particular way. For instance, any data can be utilized as an output to an output interface (e.g., monitor or other computational system) or utilized as an input for any other process.

While specific computational processing systems are described above with reference to FIG. 8, it should be readily appreciated that computational processes and/or other processes utilized in the provision of spatial cell alignment with various embodiments of the disclosure can be implemented on any of a variety of processing devices including combinations of processing devices. Accordingly, computational devices in accordance with embodiments of the disclosure should be understood as not limited to specific computational processing systems and/or cellular spatial alignment applications. Computational devices can be implemented using any of the combinations of systems described herein and/or modified versions of the systems described herein to perform the processes, combinations of processes, and/or modified versions of the processes described herein.

### Examples

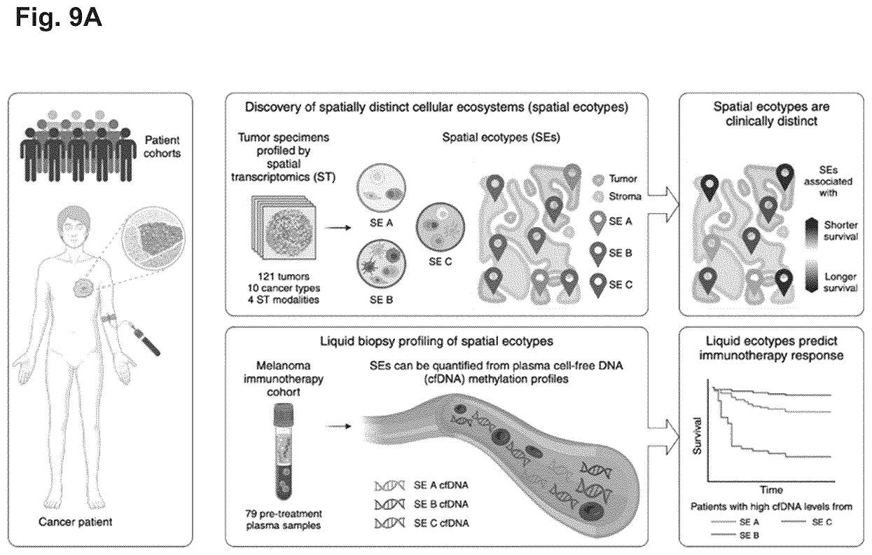

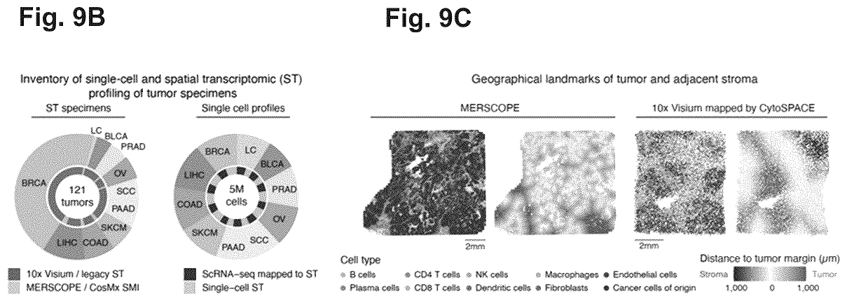

The embodiments of the disclosure will be better understood with the several examples provided within. These examples describe implementation of exemplary computational systems and methods yield an understanding of tissue SEs from assessment of omic data (FIG. 9A). Although the described examples focus on SEs solid cancer tissue, the examples provide proof of principle that the SEs can be identified from any solid tissue. The examples utilize multiple omic types (e.g., gene expression, methylation patterns) to asses composition of SEs, establishing that any omic that affects or is affected by cell state can be utilized within the various systems and methods. Also, the examples further establish that SEs can be predicted from cell-free biomolecules collected from plasma, which establishes that any cell-free source of omic data can be utilized as appropriate to the assessment being performed (e.g., urine for bladder cancer, stool for colorectal cancer, saliva for mouth cancer, etc.). The examples also describe the diagnostic ability of identifying SEs to predict patient survivability and treatment response. As can be readily appreciated, the examples establish that the described systems and methods provide a practical means for assessing medical conditions, patient outcomes, and therapy response such that an appropriate set of diagnostic procedures and treatment regimens can be determined and administered.

### Spatially Constrained Cellular Plasticity in the TME

High-resolution molecular profiling of human tumors has revealed considerable plasticity among immune, fibroblast, and endothelial subsets in the tumor microenvironment. For example, CD8 T cells preferentially express GZMB when localized to the tumor core but preferentially express GZMK when localized to the periphery. However, no study has systematically examined the interplay between TME cell states and spatial context across the entire transcriptome, both for diverse malignancies and cell types.

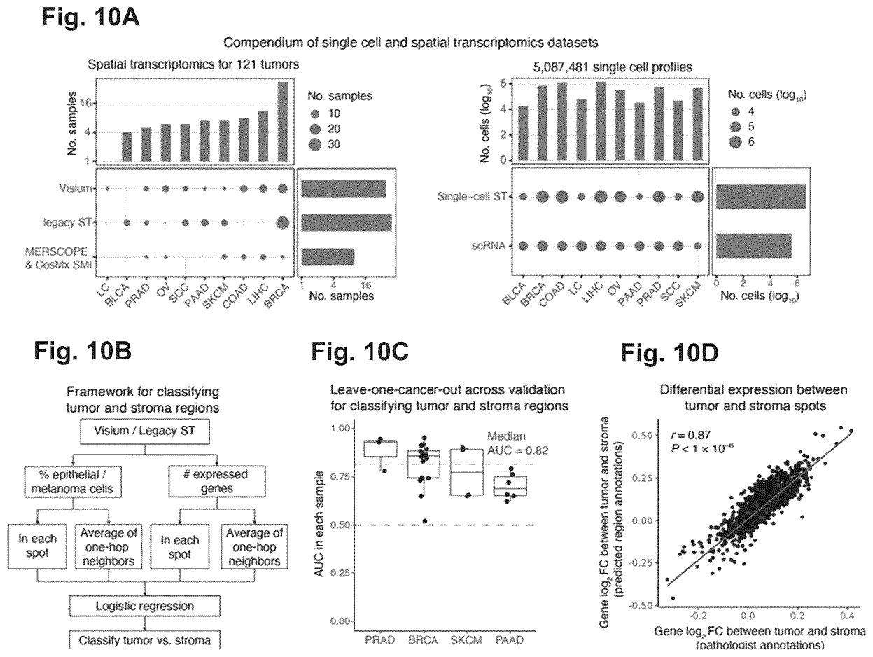

To gain insight into this question, and as a critical first step toward charting spatial ecotypes, we began by assembling a large compendium of ST data covering 121 primary human tumor specimens from two major classes of malignancy: carcinoma—the most common cancer type—and melanoma. Collectively, this dataset spans 10 distinct neoplasms (melanoma and nine carcinomas), 15 independent studies, and four ST platforms, including bulk (10× Genomics Visium and legacy ST) and single-cell (Vizgen MERSCOPE and NanoString CosMx SMI) ST data (FIG. 9B, 10A). We also curated single-cell RNA sequencing (scRNA-seq) atlases for the same 10 malignancies, spanning 281 k cells from 135 tumor samples (FIG. 10a). With CytoSPACE, we then comprehensively integrated scRNA-seq and ST data in this compendium. This allowed us to overcome the modest spatial resolution and gene recovery of bulk and single-cell ST platforms, respectively, while also leveraging single-cell ST data when available (Methods).

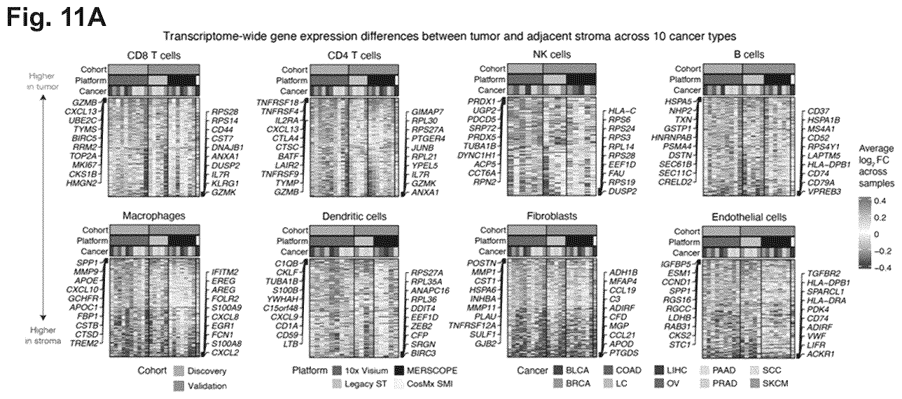

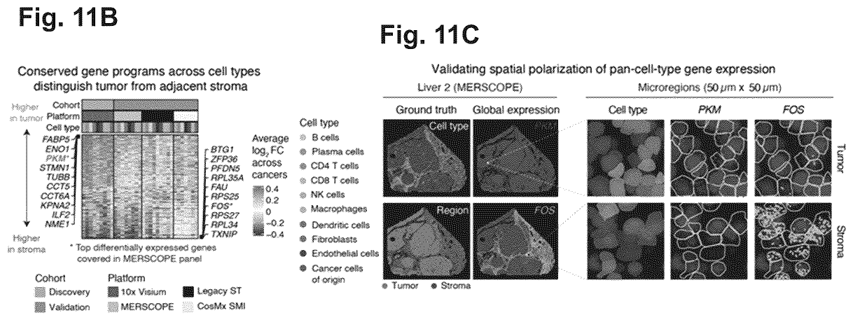

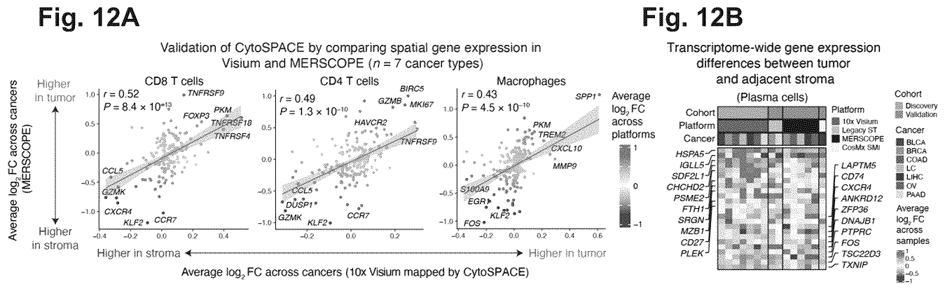

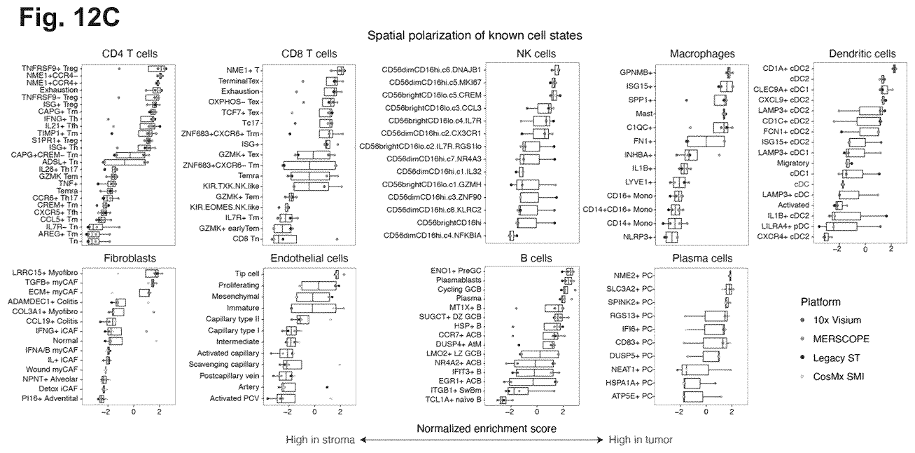

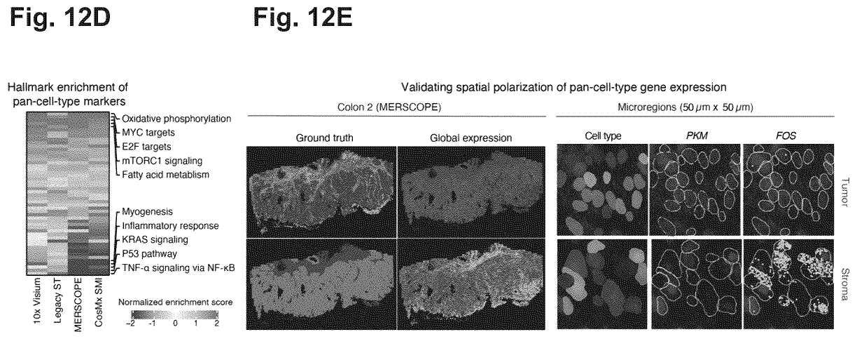

The resulting multi-cancer geographic atlas consisted of 5.6M spatially resolved transcriptomes at cell type resolution. Given the breadth and depth of this dataset, we started with a supervised analysis to probe differences in gene expression between two disparate geographic landmarks: the tumor core and the adjacent stroma (FIG. 9C, 10B-10D). Using Visium data as a discovery cohort (n=54 tumors) and the remaining ST data for validation (n=67 tumors), we identified substantial pan-cancer variation in the gene expression programs of nine major TME cell types: B cells, plasma cells, CD8 T cells, CD4 T cells, NK cells, monocytes/macrophages, dendritic cells, endothelial cells, and fibroblasts (FIG. 11A, 12A-12C). For instance, we readily confirmed T cell and macrophage genes with known spatial polarity, including GZMB and SPP1 in the tumor core and GZMK and FOLR2 in the periphery, respectively. We also identified genes with previously unknown regional specificity. For example, PRDX1 (encoding peroxiredoxin-1), which increases resistance to oxidative stress and enhances anti-tumor activity in PD-L1-CAR NK cells46, emerged as a top marker of tumor-associated NK cells. Additionally, we observed widespread variation in endothelial cells, fibroblasts, dendritic cells, B cells, and plasma cells (FIG. 11A, 12B,12C).

Surprisingly, many transcripts also showed geographic heterogeneity independent of TME cell type or malignancy, revealing broad metabolic rewiring in the tumor core and upregulation of TNF-α signaling in the periphery (FIG. 11B, 12D). For example, among the leading genetic markers of tumor and adjacent stroma in the Visium discovery cohort, the top two genes profiled by MERSCOPE—PKM (a glycolytic enzyme47) and FOS (a key target of TNF-α48)—exhibited robust patterns of regional variation, respectively. This was true at multiple scales, from individual mRNA molecules to entire tumor specimens (FIG. 11C, 12E).

Thus, through integrative analysis of single cell and spatial transcriptomics data across 10 human malignancies, we identified a rich tapestry of conserved regional plasticity in the TME.

### Geospatial Map of Multicellular Programs Across Cancers

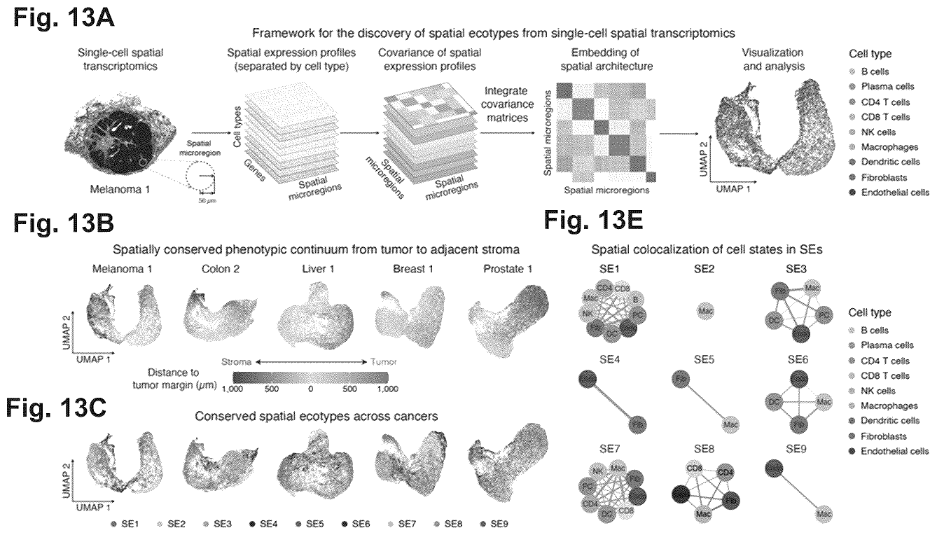

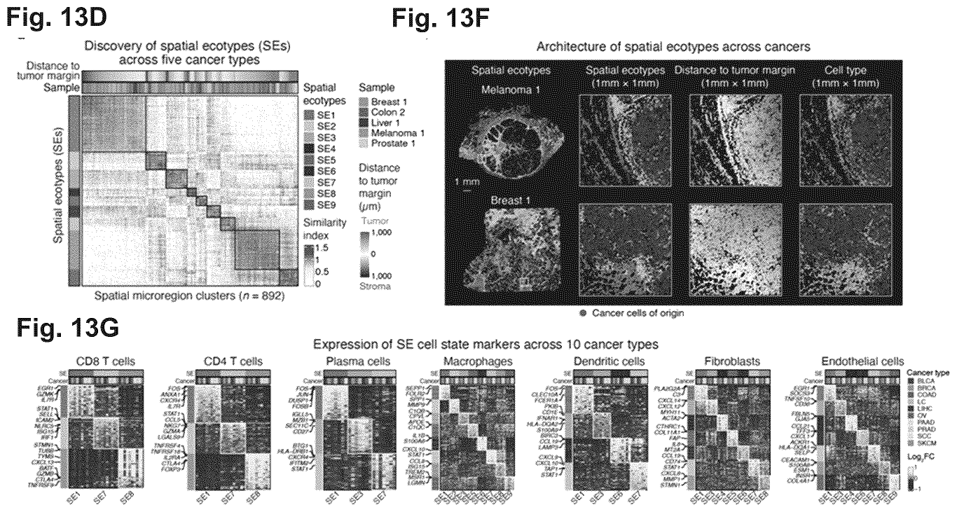

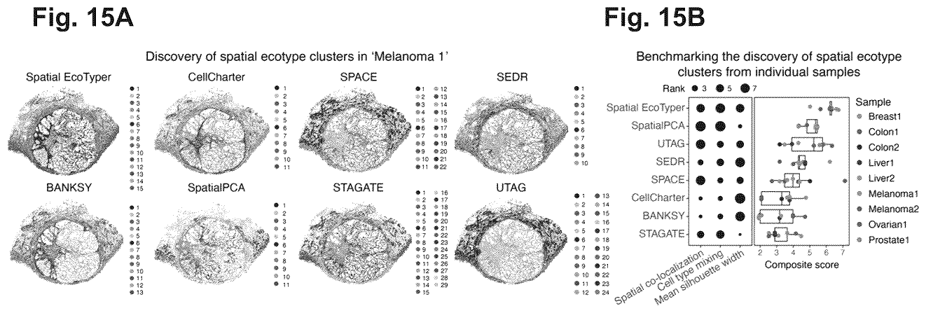

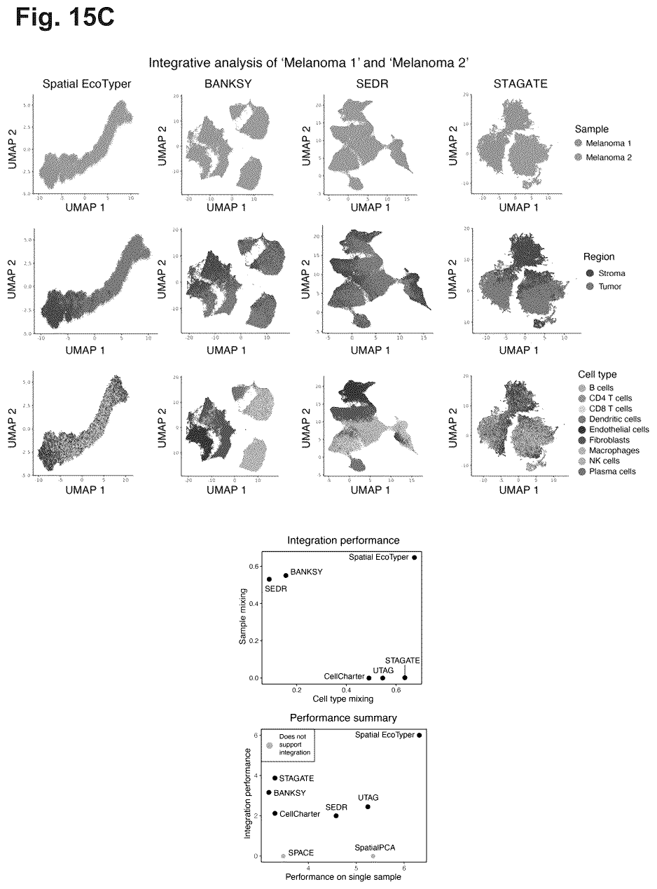

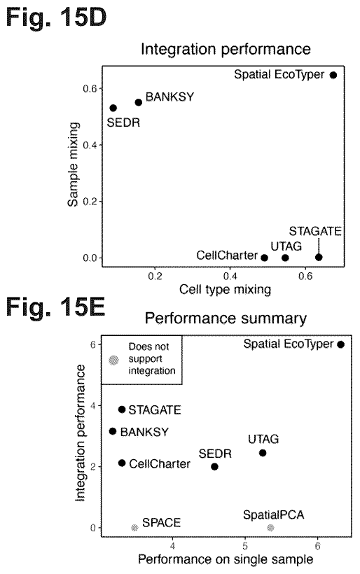

We next sought to deeply characterize the relationship between transcriptional programs and spatial coordinates for all nine TME cell types simultaneously, both across malignancies and without the constraint of supervised analysis. However, doing so has been challenging, as existing methods either ignore spatial information or cannot effectively integrate across tumor samples and cancer types. To address these issues, we developed Spatial EcoTyper, a machine learning framework that generates a unique data representation in which cell type-specific gene expression programs (GEPs) are “fused” across samples into a common, spatially informed embedding (FIG. 13A, 14). By integrating GEPs with similar spatial coordinates while balancing contributions across multiple cell types, this approach—which draws inspiration from multi-omic data fusion methods—is not only ideally suited for SE detection, but also outperforms previous approaches (FIG. 15). Once defined, SEs can be profiled at scale, whether from bulk, single-cell, or spatial expression data, using a specialized variant of non-negative matrix factorization (NMF)50.

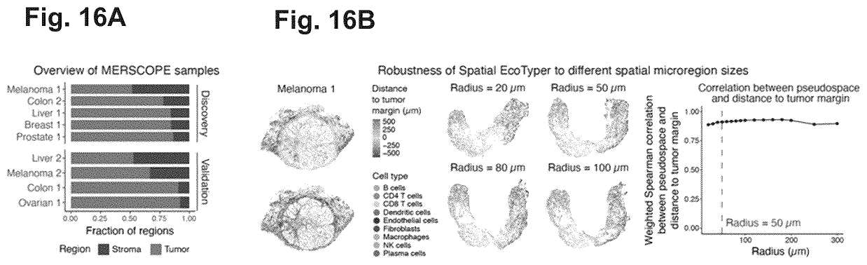

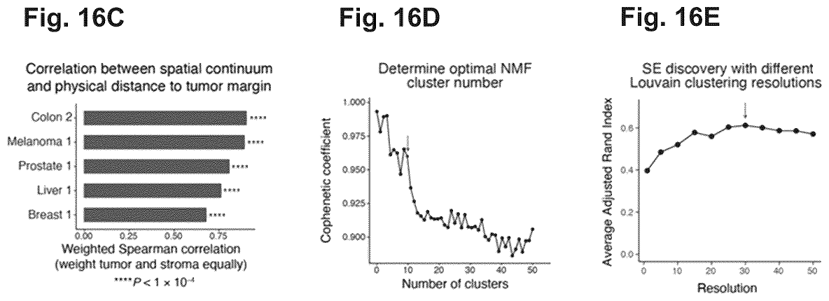

To test this strategy, we first analyzed diverse tumor specimens individually. To do so, we assembled a discovery cohort consisting of five formalin-fixed paraffin-embedded (FFPE) samples encompassing >800 k TME cells profiled by MERSCOPE in four distinct carcinomas (breast, colon, liver, prostate) and one melanoma, each with at least 5% of TME cells derived from the tumor mass or adjacent stroma (FIG. 16A). To focus on robust spatial trends while overcoming sparsity, we aggregated single-cell expression data by cell type into spatial microregions of predefined radius (50 μm) (FIG. 16B). We then applied Spatial EcoTyper to generate sample-specific embeddings.

Strikingly, when visualized using UMAP, each embedding organized into a phenotypic continuum, with individual microregions tracing a continuous trajectory from the tumor core to the adjacent stroma (FIG. 13B, 16B). This ordering was highly statistically significant (P<10-4), independent of malignancy, and robust to spatial resolution when considering microregions of diverse radii (from 20 to 100 μm) (FIG. 16C). Thus, multicellular programs in the TME appear to vary in a granular manner, reflective of their physical distance to the tumor margin.

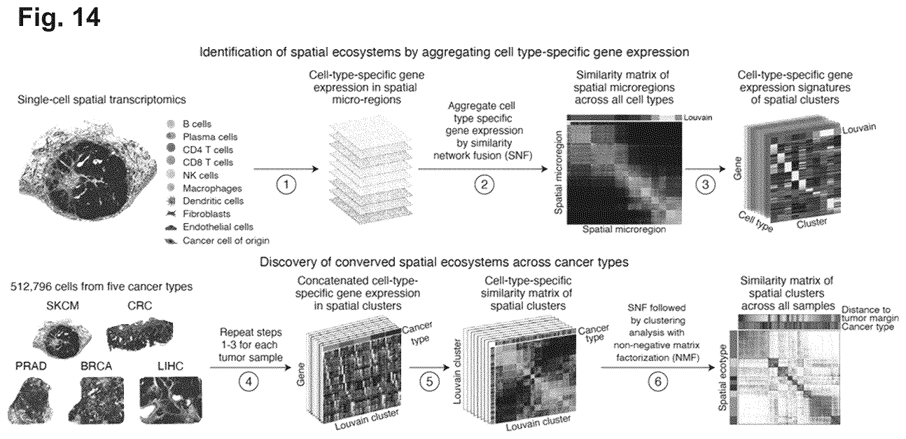

To determine whether these trajectories also reflect shared variation across cancers, we next repeated our analysis by jointly considering all samples. Using a combination of Louvain clustering, marker gene identification, and network fusion (FIG. 14), Spatial EcoTyper integrated all cell types and ST samples into a common embedding containing 41 k spatial microregions. To delineate ecotypes, we applied NMF to this embedding, revealing 9 clusters with strong co-association and mutual avoidance (FIG. 13C, 13D) that were robust to variation in key input parameters (FIG. 16B-16E). We termed these clusters “spatial ecotypes” and numbered them SE1 to SE9 according to their average physical distance from the tumor margin (FIG. 13D, 17A).

### Spatial Ecotypes are Conserved Units of TME Organization

To authenticate these results, we next assessed SE recovery in a validation cohort comprised of 100 held-out ST and 135 scRNA-seq samples from melanoma and nine types of human carcinoma. As part of this process, we defined SE-enriched cell states (n=38) using a supervised variant of NMF applied to the discovery cohort (FIG. 13E, 17B). We then quantified SEs and their corresponding cell states in the validation cohort using a previously established approach.

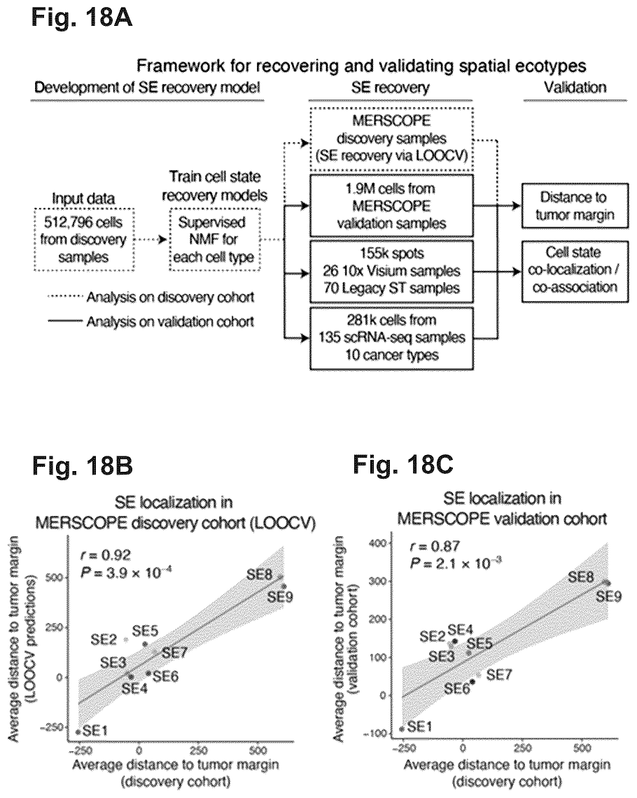

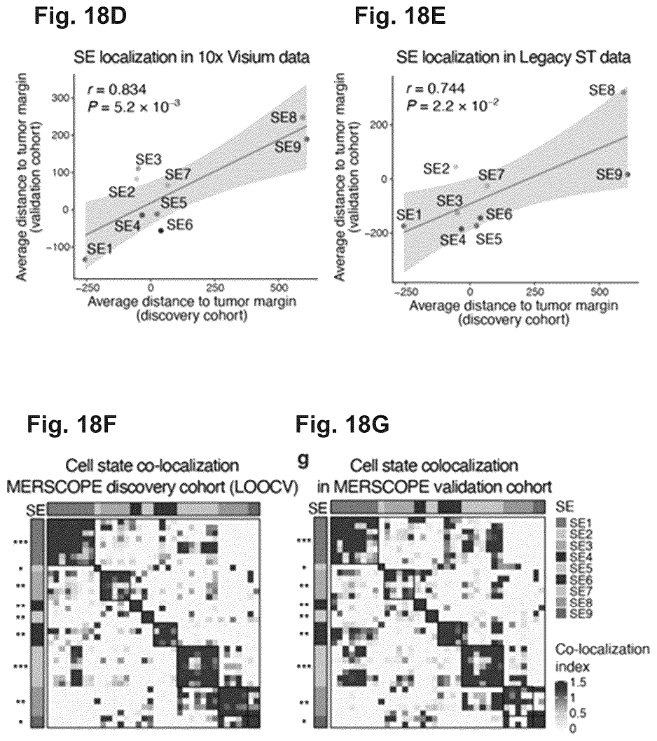

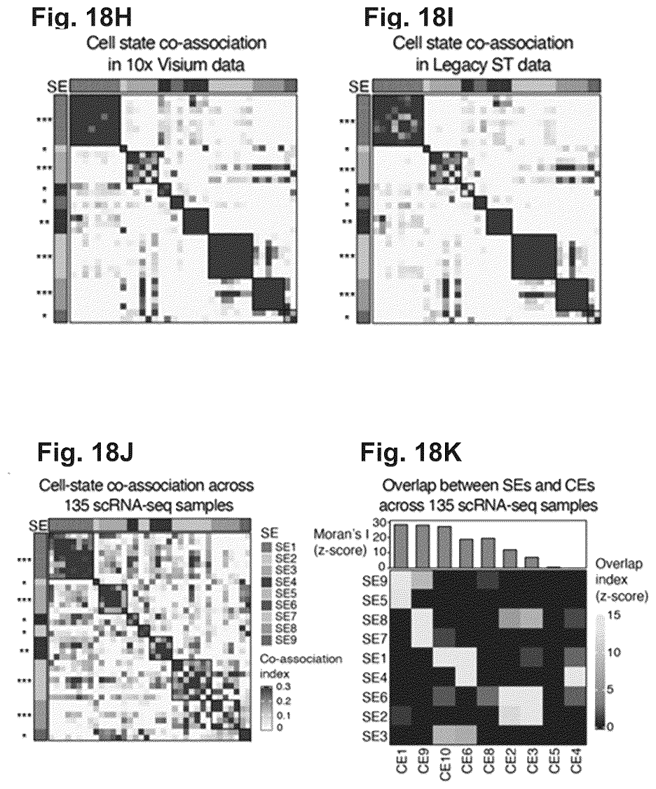

We undertook two key experiments to evaluate SE generalizability (FIG. 18). First, we asked whether SEs predicted in held-out data recapitulate their expected distances to the tumor margin. Indeed, in 100 tumor samples profiled by ST (4 by MERSCOPE, 26 by Visium, 70 by legacy ST), estimations of physical distance to the margin were remarkably concordant with expectation (FIG. 18B-18E). Next, we asked whether cell states of a given SE cooccur more strongly than expected by random chance. Indeed, by predicting cell state frequencies and their correlation patterns in 235 tumors profiled by either ST (n=100) or scRNA-seq (n=135), all SEs were readily detectable (FIG. 18F-18J). Thus, SEs exhibit conserved biogeographic features and robust cell-state co-association relationships.

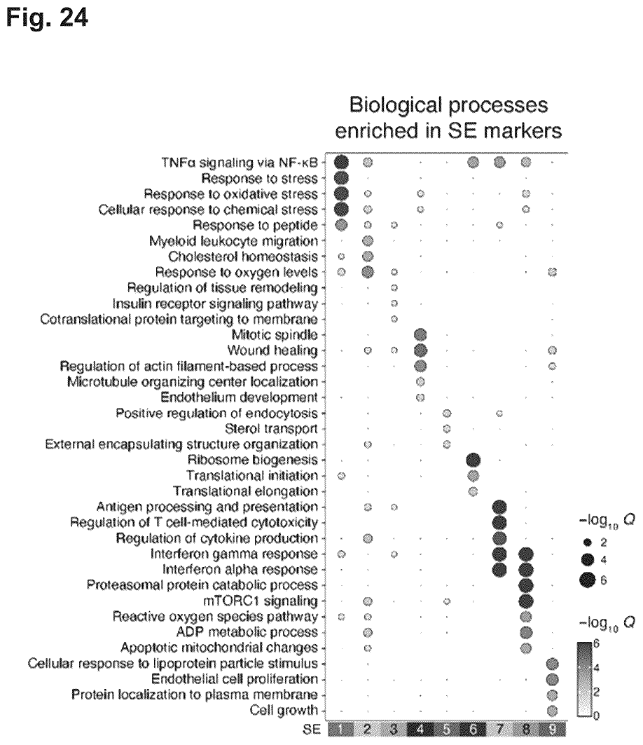

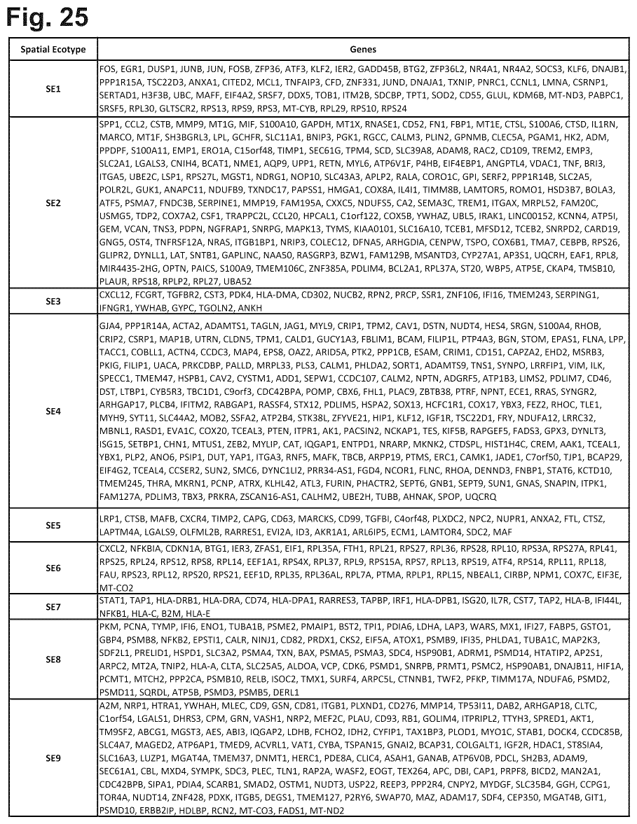

Having demonstrated the extensibility of SEs to diverse cancer types and platforms, we next explored their cellular programs across 10 malignancies profiled by scRNA-seq. Selected cfRNA Marker genes for each SE cell state are shown in FIG. 25. In addition, a subset of these genes also distinguished SEs independently of TME cell type or malignancy. Such genes, termed consensus markers, were generalizable to single-cell-scale ST data and associated with distinct functional programs, establishing them as robust molecular hallmarks of SE biology. Any of these genes can be interrogated in a cfRNA assay as described herein. See, e.g., FIG. 24.

Combining these data with cellular and spatial features, it was determined that SE1—the most stromal-enriched ecotype—is associated with pervasive expression of early response genes including FOS and EGR1. These genes, previously attributed to adjacent normal tissue using bulk transcriptomics, have not been previously systematically delineated across spatially resolved TME subsets. Starting with SE1—the most stromal-enriched ecotype—we identified a range of co-associated cell states previously ascribed to adjacent normal tissue (FIG. 13E-13B, 17B). These include naïve and central memory T cells (GZMK+, IL7R+, CXCR4+)37, FOLR2+ macrophages, cDC2 cells (CLEC10A+, FCER1A+), adventitial fibroblasts (C3+), and activated capillary cells (EGR1+ endothelial cells). Conversely, analysis of SE9, the spatial ecotype most strongly enriched in the tumor core, identified two co-localized cell states previously attributed to the tumor mass and with endothelial cell proliferation: TREM2+ tumor-associated macrophages (TAMs) and tip cells underlying neovascularization (INSR+ endothelium) (FIG. 13G). FIG. 25 shows a representative group of SE consensus markers associated with various biological processes. Consensus SE-specific markers were defined as genes recurrent across ≥80% of SE-specific cell states. Given that SE3 had <20 genes following that selection protocol (n=16), we augmented it by including genes at a relaxed cell-type-conservation threshold of 60%, selected in order of decreasing conservation across cancer types, until a 20-marker minimum was satisfied. For SE2, which comprises a single cell state, markers were defined as genes that are significant in at least three cancer types. To ensure specificity, genes associated with multiple SEs were assigned to the SE with the highest number of significant cancer types. In cases where markers overlapped between SE2 and other SEs, genes were preferentially assigned to non-SE2 states. These markers are representative, as methods of the invention are applicable to any tissue or liquid-based markers.

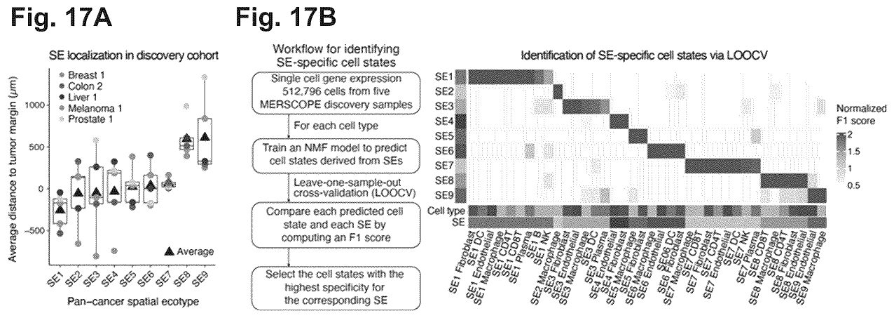

Unlike SE1 and SE9, the majority of SEs were found to preferentially localize within 250 μm of the tumor-stroma margin (FIG. 17A). These include six SEs composed of unique plasma cell, macrophage, dendritic cell, fibroblast, and/or endothelial cell states, each with strong conservation across carcinomas and melanoma (FIG. 13E-13G). For example, we identified a large multi-phenotypic hub (SE3) comprised of IGLL5+ plasma cells, C1QB+ macrophages38, CD1E+ dendritic cells, CXCL12+ cancer-associated fibroblasts (CAFs), and CD36+ endothelial cells. We also identified bi-phenotypic hubs comprised of distinct myeloid and/or stromal cell states (SE4, associated with wound healing is defined by MYH11+ myofibroblasts and hypoxia-associated FBLN5+ endothelial cells; SE5, associated with immunosuppression, is composed of FAP+ cancer-associated firbroblasts, APOE+M2-like macrophages, and TAMs38. (FIG. 13G). These and other margin-enriched ecotypes reveal extensive plasticity and stereotypic organization along this phenotypic transition.

We also discovered two proinflammatory spatial ecotypes with elevated interferon signaling but distinct regionalization. SE7, which is largely restricted to the tumor margin, encompasses co-associated cell states from a total of eight lymphoid, myeloid, and stromal lineages, each with elevated expression of STAT1, and consensus genes associated with antigen processing and presentation. SE8, which is generally localized to the tumor core, consists of GZMB+ T cells8, 37, CCL8+ TAMs, CXCL8+ CAFs, and CEACAM1+ endothelial cells with consensus genes enriched in elevated metabolism (FIG. 13E-13G). Beyond the tumor microenvironment, it was determined that spatially adjacent non-TME cells, including malignant subsets, also express consensus markers for a subset of ecotypes. This suggests that SEs can participate in larger multicellular assemblies with shared spatial programs.

Given that SEs are spatially defined, we next compared them against carcinoma ecotypes (CEs) identified in recent work by bulk RNA-seq deconvolution. We found that CEs with tighter spatial aggregation patterns, as determined by Moran's I, were more likely to have at least two distinct SE counterparts (FIG. 18K). For example, CE9—which previously outperformed competing measures for predicting benefit from immune checkpoint inhibition (ICI)8—is actually a composite of SE7 (margin-associated) and SE8 (intra-tumoral). Thus, by leveraging Spatial EcoTyper, we discovered numerous local microenvironments that were previously undetectable without high-resolution spatial analysis.

### Clinical Significance of Spatial Ecotypes

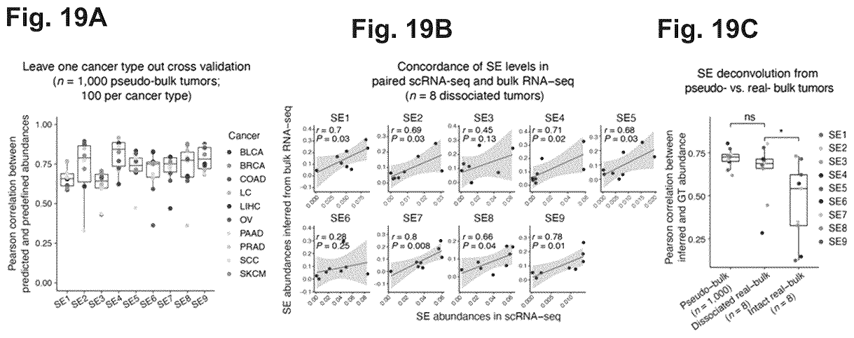

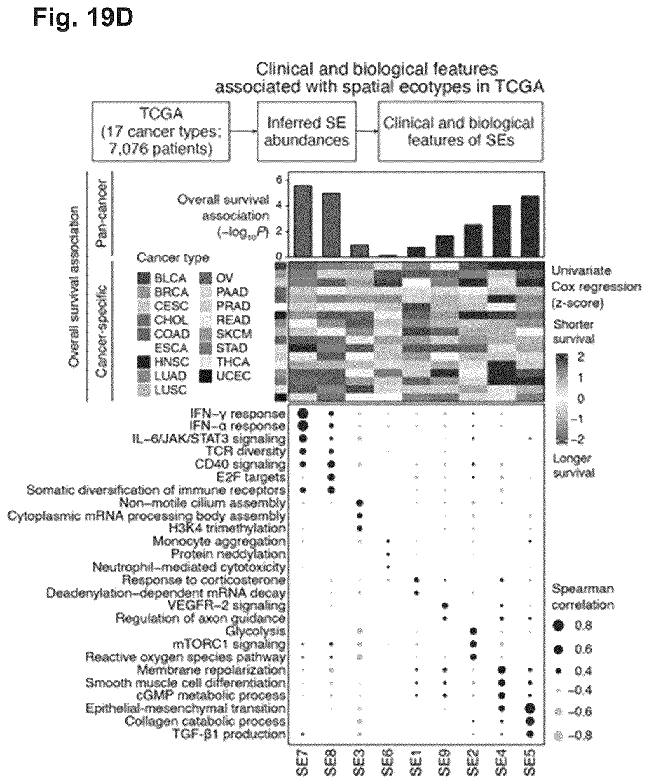

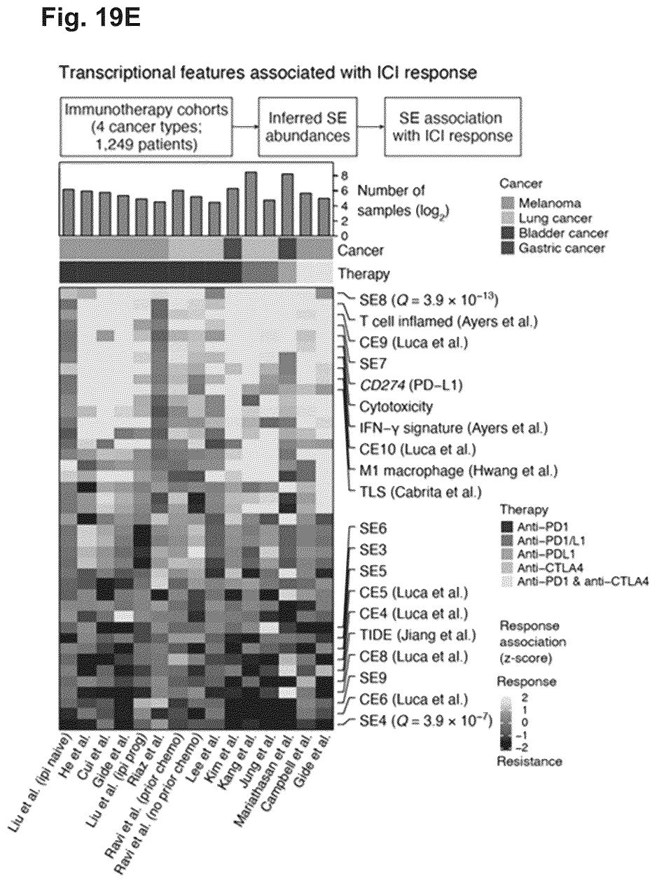

To characterize the potential clinical relevance of SEs, we next devised a gene expression deconvolution strategy to determine SE composition at massive scale. Using scRNA-seq data from 10 cancer types, we leveraged 1,000 pseudo-bulk tumors with fully defined SE composition to train a supervised NMF model for quantifying SE content. Following ten-fold cross-validation, we observed high accuracy for deconvolving SE levels in reconstituted tumors from held-out cancers (FIG. 19A, 20A, 20B).

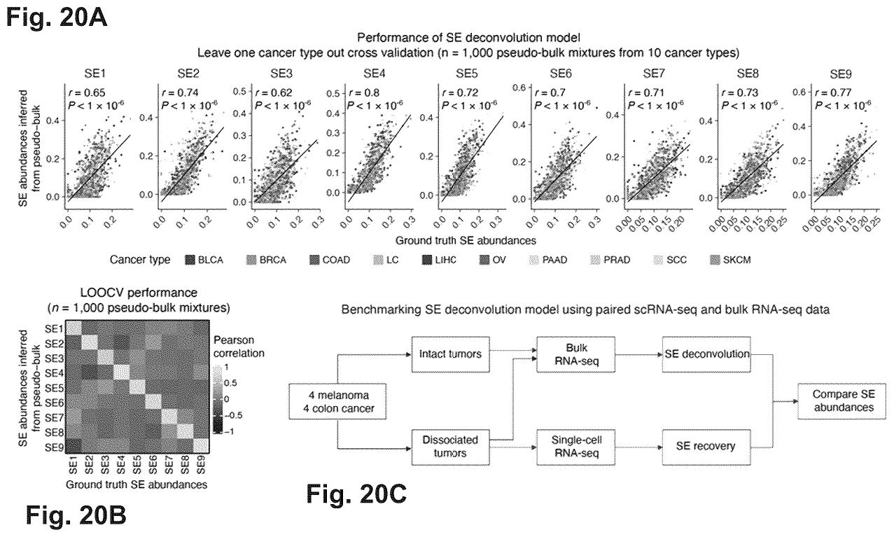

We then assessed the model's capability to recover SE levels in real bulk tumors by generating paired bulk RNA-seq and scRNA-seq profiles of eight tumor specimens from patients with melanoma or colorectal carcinoma (FIG. 20C). To account for dissociation-induced distortions, we sequenced mRNA from two conditions: (i) intact tumor biopsies and (ii) the same cellular digests used for scRNA-seq. Consistent with expectation, we found that deconvolution of the latter achieved the strongest concordance with SE composition in paired scRNA-seq data (FIG. 19B, 19C). Moreover, nearly every ecotype was significantly correlated with ground truth, validating our approach (FIG. 19B).

To extend this analysis to intact specimens, we analyzed paired bulk RNA-seq and 10× Visium data of tumor samples from 42 patients spanning breast cancer, colorectal cancer, lung cancer, ovarian cancer, and pancreatic cancer from the Human Tumor Atlas Network (HTAN). To compute SE abundances from each Visium sample, we aggregated deconvolution results from individual spatial spots. We found that all nine SEs were significantly and specifically correlated between platforms. Additionally, performance was generally independent of cancer type or disease state, and consistent with other validation scenarios, emphasizing robustness.

To further evaluate SE recovery from bulk ST data, we performed spatial RNA sequencing with Visium and multiplexed RNA imaging with MERSCOPE on adjacent melanoma sections. Across SE-assigned cells (MERSCOPE) within 815 overlapping Visium spots, SE microarchitectures were significantly correlated between modalities. Similar results were obtained from publicly available data of paired Visium and Visium HD profiles of a colon cancer specimen. Collectively, these data demonstrate the promise of Spatial EcoTyper for high-resolution digital profiling of TME spatial ecosystems from bulk expression data.

Having established the feasibility of SE deconvolution, we next applied our approach to 7,076 clinically annotated bulk RNA-seq profiles of melanomas and 16 types of carcinoma from The Cancer Genome Atlas (TCGA). Two-thirds of evaluable SEs (6 of 9) were significantly prognostic for overall survival when assessed across cancers after multivariable adjustments for age and sex (FIG. 19D). Among them, SE5, a tumor margin-enriched ecotype associated with elevated epithelial-mesenchymal transition and TGF-β1 production, emerged as the leading determinant of shorter survival time (FIG. 19D). Conversely, proinflammatory ecotypes SE7 and SE8, distinguished by TME location (margin and tumor core, respectively) and pathway activity (interferon and CD40 signaling, respectively) (FIG. 19D), were significantly predictive of longer survival time. While most cancer types showed consistent survival patterns, we observed reciprocal associations for prostate, esophageal, and pancreatic cancers. These inversions were consistent with factors that hinder anti-tumor immunity, including lower expression levels of MHC-I and MHC-II in prostate and esophageal carcinomas, and a dense desmoplastic stroma in pancreatic cancer.

Given previous data linking CE9 and other carcinoma ecotypes to immunotherapy response, we next wondered whether SEs could enhance ICI outcome stratification. To this end, we assembled bulk tumor RNA-seq profiles of 1,249 pretreatment tumors spanning four cancer types (melanoma and three carcinomas) and 12 studies, all with annotated response outcomes to ICI monotherapy (anti-PD-1 or anti-PD-L1) or combination therapy (anti-PD-1/anti-CTLA-4) (Supplementary Table 13). We then enumerated SEs, CEs, and 22 additional potential correlates of ICI response. CE9 was again a strong correlate of ICI benefit, corroborating previous findings in a greatly expanded meta-analysis (FIG. 19E). Nevertheless, SE8—a daughter ecotype of CE9 with strong intra-tumoral localization—outperformed it (FIG. 19E, 17A, 18K). SE7, another daughter of CE9 with localization to the margin, showed nearly comparable performance (FIG. 19E, 17A, 18K). It also surpassed SE8 in predicting ICI benefit when applied to melanoma alone. Moreover, SE4, a myofibroblast and hypoxia-associated endothelial cell ecotype, emerged as the top correlate of ICI resistance (FIG. 19E). Thus, by coupling transcriptional and spatial variation into cohesive cellular assemblies, SEs have potential for improved clinical outcome prediction.

To extend this analysis to established ICI biomarkers, we examined 465 patients across all datasets with available tumor mutational burden (TMB) data (non-small cell lung cancer, bladder cancer, and melanoma), using CD274 expression as a surrogate for PD-L1 levels. Across datasets and patients, SE7 and SE8 outperformed TMB and CD274 in predicting overall survival in multivariable models, with all three ecotypes including SE4 showing superior performance in univariate models.

### Spatial Ecotype Profiling by Liquid Biopsy

To fully exploit spatial ecotypes, it will be important to address key barriers that limit their broad clinical applicability. These include tumor sampling bias in metastatic disease, tumor geographic heterogeneity, and the impracticality of acquiring serial tumor biopsies for longitudinal analysis. Liquid biopsies represent a promising means of overcoming such challenges but have not been previously applied to detect multicellular ecosystems. Given the promise of cell-free DNA (cfDNA) methylation profiling for deciphering cell-of-origin contributions to peripheral blood plasma, we hypothesized that a cfDNA methylation approach might allow SE levels to be quantified in a more systemic, unbiased, and accessible manner.

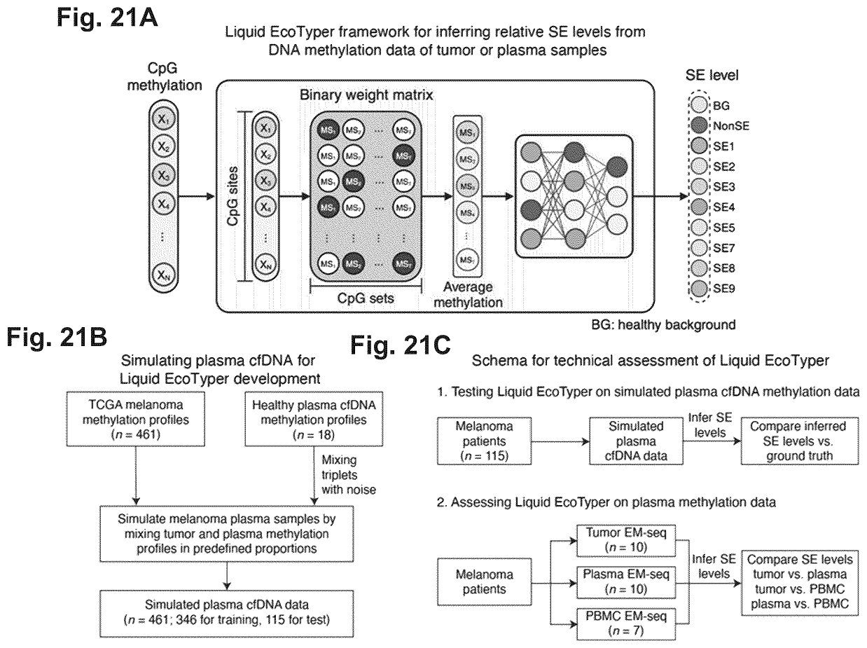

To explore this hypothesis, we designed and trained a novel deep learning framework, termed Liquid EcoTyper, to infer SE levels from CpG methylation data (FIG. 21A). Our approach is based on a binary neural network architecture that learns discriminatory CpG sets (akin to gene sets) to quantify SE levels, non-SE tumor content, and healthy cfDNA background (FIG. 21A). By learning CpG sets via a binary network, our design enforces regularization, facilitates resistance to dropout and sparsity of individual CpGs, and allows all CpG signatures to be seamlessly extracted without feature inference strategies, providing complete transparency.

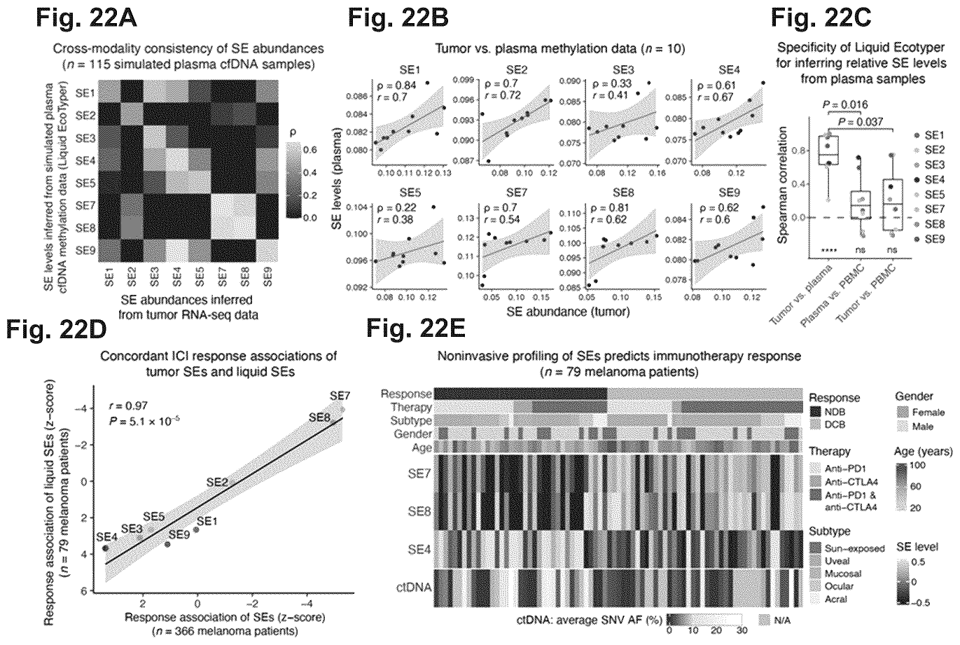

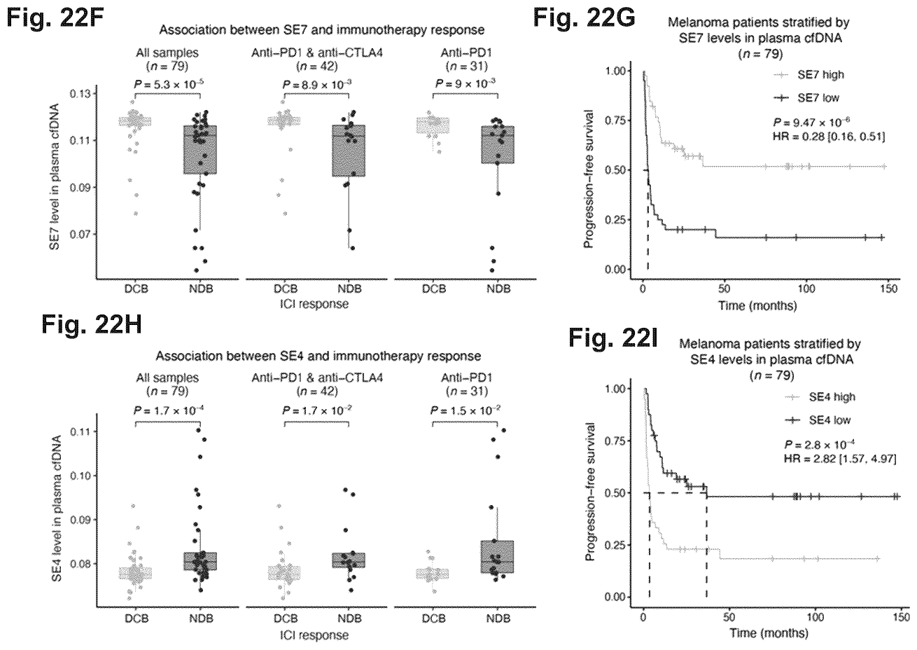

To implement Liquid EcoTyper, we focused on metastatic melanoma, a cancer type for which ICI therapy is standard-of-care and for which extensive multimodal genomic data, including datasets with known ICI outcomes, are publicly available. We then compiled 461 melanomas from TCGA with paired 450 k methylation array data and bulk RNA-seq profiles59, the latter of which served as “ground truth” data for SE composition (Methods). To boost specificity, we generated NEBNext Enzymatic Methyl-seq (EM-seq) profiles of cfDNA collected from 23 healthy controls. We then simulated cfDNA from melanoma patients by combining CpG methylation profiles from tumor genomic DNA and healthy cfDNA in defined proportions (FIG. 21B). By evaluating the model on 115 simulated melanoma cfDNA profiles held out from training, we observed strong specificity and linearity in resolving nearly all SEs using CpG methylation signatures (FIG. 22A). Our approach also readily extended to 13 types of primary carcinoma in proof-of-principle analyses, underscoring generalizability.

To extend this analysis to real plasma while accounting for key confounding variables, we next performed whole-genome EM-seq profiling of tumor genomic DNA and plasma cfDNA, all of which were concurrently isolated from 10 melanoma patients with advanced disease (FIG. 21C). Peripheral blood mononuclear cell (PBMC) DNA was also extracted from 7 of these patients for EM-seq profiling. To emulate clinical conditions, we limited our analyses to clinically practical cfDNA mass input amounts (<10 ng per subject). We then applied Spatial EcoTyper to ST data and Liquid EcoTyper to all sequenced methylomes.

Strikingly, SE levels determined by our approach were well-correlated between plasma and tumor compartments for nearly all evaluable ecotypes (FIG. 22B). Significant correlations were observed for all but one ecotype profiled by spatial transcriptomics and plasma EM-seq; and for all but one SE profiled by tumor and plasma EM-seq, with no significant difference between modalities. In line with this, differences in cfDNA abundance between two SEs with significant but reciprocal associations with ICI response in melanoma-SE7 and SE4-were reflective of their corresponding levels in situ by spatial transcriptomics. These results were highly specific, as correlations between inferred SE levels in PBMCs and tumors from the same patients were substantially lower and not significantly different from 0 (FIG. 22C). The same was true for inferred SE levels in PBMCs versus matched plasma samples (FIG. 22C). This suggests that plasma-derived SE signal is largely specific to metastatic melanomas and not simply an artifact of DNA shed from circulating leukocytes.

Given these results, we next explored the clinical potential of liquid SE profiling in 79 patients with metastatic melanoma treated with ICI monotherapy (anti-PD-1 or anti-CTLA-4, n=37 patients) or combination therapy (anti-CTLA-4 and anti-PD-1, n=42). To this end, we generated whole-genome methylation (EM-seq) profiles of clinically obtainable quantities of pretreatment plasma cfDNA from all patients. We also performed targeted sequencing of pretreatment circulating tumor DNA (ctDNA) for 60 patients and tumor mutational burden (TMB) profiling for 38 patients as a comparator.

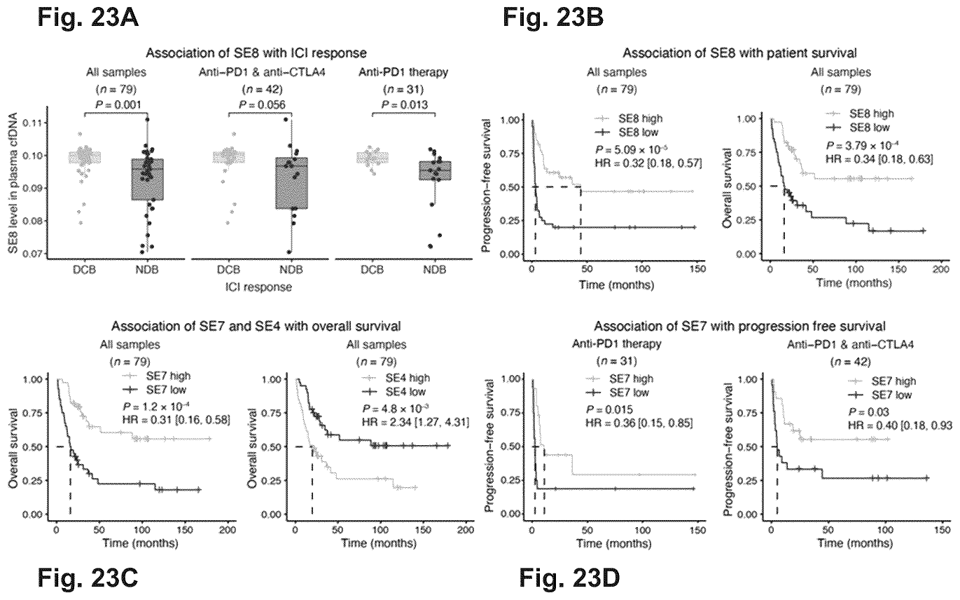

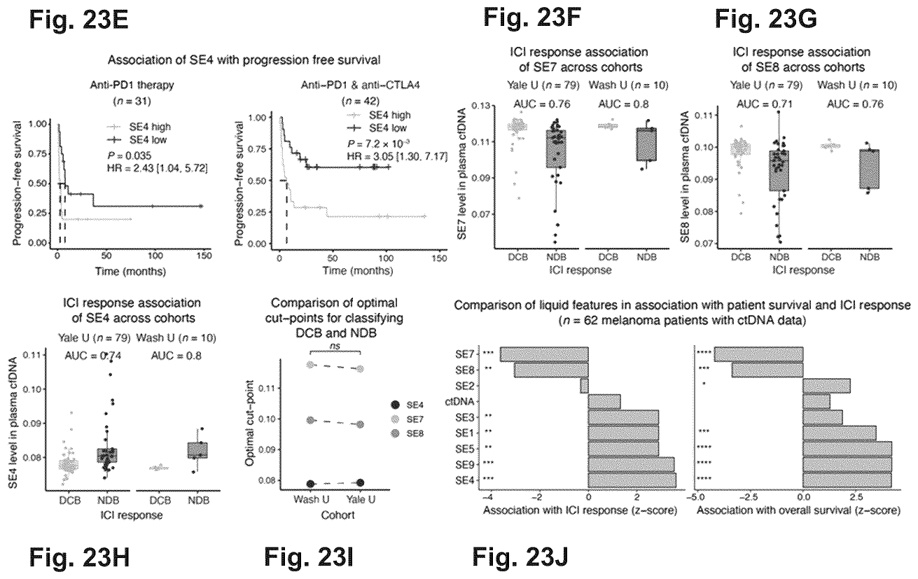

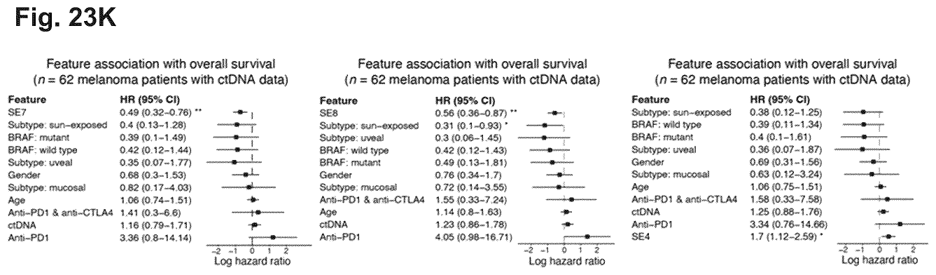

When evaluated across melanoma patients, ICI response associations for spatial ecotypes were nearly perfectly correlated between plasma-derived and bulk tumor RNA-seq-derived measurements (FIG. 22D). This consistency was remarkable given data generated from disparate cohorts, tissue compartments, and modalities. In particular, elevated SE7 and SE8 levels determined in pretreatment plasma were strongly associated with future durable clinical benefit (DCB) and longer progression-free survival (PFS) (P<0.0001 and HR<0.32 for both) whereas higher SE4 levels forecasted ICI resistance and shorter PFS (P<0.0001, HR=2.82) (FIG. 22D-22I, 23A-22E). These relationships were not only consistent with our previous findings from bulk RNA-seq data, but were also robust across various ICI therapy types, melanoma subtypes, sex, and age. Moreover, our findings were maintained in an independent cohort of 10 melanoma patients from another institution, with no significant difference in optimal cut points for dichotomizing SE levels in relation to ICI response (FIG. 23F-23J), with consistent relationships between model-derived CpG signatures and plasma-derived SE levels across cohorts.

In contrast, higher pretreatment ctDNA levels were only modestly associated with ICI resistance and shorter OS, in line with previous studies (FIG. 23J). Moreover, baseline levels of ctDNA were not significant in multivariable survival models incorporating key clinical indices and plasma-derived levels of SE7, SE8, or SE4 (FIG. 23K). We also interrogated tissue-based TMB and PD-L1 levels as established biological benchmarks to assess whether liquid SEs capture additional, non-redundant biology. In patients with evaluable TMB or PD-L1 levels, all three liquid SEs (SE7, SE8, or SE4) were more-significantly associated with OS regardless of multivariable adjustment. These data—coupled with complementary tissue-based findings across 465 patients and three cancer types—further demonstrate that SEs capture clinically relevant biology beyond approved ICI biomarkers.

Thus, liquid SE profiling has potential to access the TME noninvasively, infer its spatial cellular architecture, and outperform circulating tumor DNA detection for early ICI response assessment.

### Human Subjects

All human samples included in this study were collected with informed consent for research use and received approval from the Institutional Review Boards of both Yale University School of Medicine and Washington University School of Medicine, in accordance with the principles of the Declaration of Helsinki (2013). This study involves a total of 117 human subjects, divided into the following three cohorts:

Cohort 1. Intact and dissociated tumor samples were collected from eight patients (four with melanoma, four with colon cancer) at the time of surgery. Each sample underwent bulk RNA sequencing, and the dissociated tumor samples additionally underwent single-cell RNA sequencing (scRNA-seq).

Cohort 2. Tumor, peripheral blood mononuclear cells (PBMCs), and plasma cfDNA samples (10 tumor, seven PBMC, and 10 plasma cfDNA) were collected from 10 metastatic melanoma patients. An additional 23 plasma cfDNA samples were collected from healthy individuals. All samples were profiled by whole-genome Enzymatic Methyl sequencing (EM-seq).

Cohort 3. Plasma samples were collected from 79 melanoma patients who received immune checkpoint inhibitor (ICI) monotherapy (31 receiving anti-PD-1 and six receiving anti-CTLA-4) or combination therapy (42 receiving anti-PD-1/anti-CTLA-4 combination). Samples were collected before treatment initiation (before or on the first day of ICI cycle 1) and underwent whole-genome Enzymatic Methyl sequencing. ICI response was classified as either durable clinical benefit (DCB) or no durable benefit (NDB) by a board-certified medical oncologist, reflecting each patient's disease response six months after ICI initiation. Progression-free survival was determined from the start of ICI treatment.

All clinical features, including age and sex, were documented using electronic medical records from Siteman Cancer Center (Cohorts 1 and 2) and Yale Cancer Center (Cohort 3).

### Sample Processing and Sequencing

### Tumor Tissue Dissociation

Viably preserved patient tumor samples were processed by thawing material in a 37° C. water bath, washing with sterile 1×PBS to eliminate visible blood products (residues/clots), and mechanically mincing and mixing the tissue on a sterile petri dish. Minced tissue was then transferred to a 15 mL conical tube for further digestion using 3 mL enzymatic dissociation media (1 g collagenase, Sigma C5138-1G; 0.1 g DNAse I, Type IV, Sigma D5025; 10 mL HEPES, 10 mM; and 2500 U/1 L hyaluronidase, Sigma H6254 in 1 L RPMI). The tissue was agitated in dissociation media at 37° C. for 10 min, then decanted for 1 minute at room temperature. The supernatant, containing a cellular suspension of digested tissue, was transferred to 10% FBS in a fresh 15 mL tube on ice while the remaining undigested tumor tissue pellet was subjected to a second round of agitation at 37° C. for 7 min with fresh dissociation media. Following this second digestion, the supernatant was gently removed to avoid clumps and added to the same 15 mL tube of 10% FBS. The combined dissociated cell suspension was passed through a 100-micron filter and centrifuged at 500 g for 5 min at 4° C. to generate a cell pellet. The cell pellet was then washed with sterile cold 1×PBS. The final cell pellet was resuspended in 1×PBS+10% FBS for downstream use.

### Bulk RNA Sequencing

Paired intact tumor and dissociated tumor cells preserved in cryogenic conditions (Cohort 2) were thawed in a 37° C. water bath. RNA extraction was performed using the RNeasy micro kit (QIAGEN), and quality was assessed using a 2100 Bioanalyzer System (Agilent Technologies). All samples exhibited high quality for TruSeq RNA Exome analysis (DV200 >30%) and were processed using the TruSeq RNA Exome Kit (Illumina) following the manufacturer's instructions. Following hybrid capture, cDNA libraries were combined and sequenced on a NovaSeq S4 instrument (Illumina) with 2×150 bp paired end reads, targeting 40M reads per sample. Next, Salmon (v1.10.1)¹ was used to align reads to the human genome (GRCh38.p14 transcript sequences from GENCODE) followed by preprocessing with the txImport R package (v1.32.0).

### Single-Cell RNA Sequencing

Single-cell suspensions were prepared as recommended by the 10× Genomics Chromium Single Cell 5′ Reagent Kits v2 user guide. The samples were resuspended in PBS+0.04% BSA. Sample viability was assessed, and samples were adjusted for a final concentration of 1,000 cells/uL. The cDNAs were prepared after GEM generation and barcoding, followed by GEM RT reaction and bead cleanup steps. Purified cDNA was amplified for 13 cycles, then cleaned up using SPRIselect beads. cDNA concentration and quality were determined for each sample using a 2100 Bioanalyzer instrument (Agilent Technologies). cDNA libraries were prepared as recommended by the 10× Genomics v2 user guide with PCR cycles modified based on calculated cDNA concentrations. For sample preparation on the 10× Genomics platform, the Chromium Single Cell 5′ Library and Gel Bead Kit v2 (PN-1000006), Chromium Single Cell A Chip Kit (PN-120236), and Chromium 7 Multiplex Kit (PN-120262) were used. The cDNA libraries were sequenced on a NovaSeq S4 instrument (Illumina). A median of 50,000 reads/cell was targeted for each sample.

Plasma Preparation from Whole Blood

Patient whole blood was collected in vacutainer tubes containing K2 EDTA (BD Biosciences). Between 1 and 6 hours after collection, blood samples were centrifuged at 1,200 g for 10 min at 18° C. to separate out acellular plasma from cellular plasma-depleted whole blood (PDWB). The plasma layer was transferred to a new 15 mL conical tube and centrifuged a second time at 1,800 g for 10 min at 18° C. to pellet any residual cells, yielding double spun (DS) plasma. These residual cells were added to PDWB samples. The acellular supernatant containing DS-purified plasma was then pipetted to a clean 15 mL tube, mixed, and divided into 2 mL aliquots before being snap frozen in dry ice. These samples were stored at −80° C. for downstream use.

PBMC Preparation from Whole Blood

Following preparation of PDWB samples from whole blood, a density gradient centrifugation protocol using Lymphoprep (STEMCELL Technologies) and SepMate tubes (STEMCELL Technologies) was applied to harvest PBMCs. In brief, 15 mL of Lymphoprep was added to a 50 mL SepMate tube, then patient PDWB was diluted with an equal volume of 1×PBS and transferred to the SepMate tube containing Lymphoprep. The PDWB Lymphoprep solution was centrifuged at 1,200 g for 10 min with an acceleration of 5 and the brake off. After a top waste layer of supernatant was removed, the middle phase, enriched with PBMCs, was recovered and washed with 40 mL of 1×PBS by centrifugation at 500 g for 7 min at 4° C. The cleaned PBMC pellet was washed a second time with 20 mL of 1×PBS and centrifuged at 500 g for 5 min at 4° C. Following the second wash, the PBMCs were counted and cryopreserved at a concentration of 10M cells per cryotube in our in-house freezing medium (90% FBS and 10% DMSO). Cryo-coolers containing PBMCs were stored at −80° C. for 24 hours and then moved to liquid nitrogen for long term storage.

### Plasma and Genomic DNA Isolation and Quantification

Cell-free DNA was extracted from plasma using the AVENIO cfDNA Isolation Kit (Roche) according to the manufacturer's instructions. Genomic DNA from tumor and PBMCs was extracted using the QIAamp DNA Micro Kit (Qiagen) according to the manufacturer's instructions and subsequently fragmented using a LE220 focused ultrasonicator (Covaris). DNA was then quantified by the Qubit dsDNA High Sensitivity Assay (ThermoFisher) to determine yields. Lastly, DNA fragment sizes were determined with a 2100 Bioanalyzer using the High Sensitivity DNA Kit (Agilent Technologies).

### Enzymatic Methyl Sequencing

Libraries were prepared using the NEBNext® Enzymatic Methyl-seq (EM-seq™) kit and unique dual index primer pairs (New England Biolabs) according to the manufacturer's instructions (NEB #E7120 S/L v6.0_3/2). Each library input contained up to 10 ng of cfDNA and up to 100 ng genomic DNA from patient samples. Unmethylated lambda phage DNA was added to evaluate the methylation conversion efficiency. Six amplification cycles were used for 100 ng of input DNA and 8 cycles for inputs under 10 ng. Libraries were sequenced using 2×150 bp paired-end reads to a target of approximately 600M reads per sample on a NovaSeq S4 instrument (Illumina).

All EM-seq reads were analyzed and processed using Serpent, an open-source and reproducible methylation analysis pipeline. In brief, raw sequencing data were quality filtered and trimmed using fastp (v0.23.2), then aligned using bwa-meth (v2.2.1). Reads were aligned to a custom GRCh38 reference that applied the NCBI U2AF1 masking file and the ENCODE DAC Exclusion List. Reads with more than 3 non-converted Cs were considered to have undergone incomplete methylation conversion and were excluded from analysis. Methylation data were extracted from aligned .bam files using biscuit (v1.3.0). Comprehensive quality control checks were conducted across all samples.

AVENIO cfDNA Sequencing

The AVENIO cfDNA library prep Kit (Roche) was utilized to prepare sequencing libraries, incorporating unique sample indexes, in accordance with the manufacturer's guidelines. In brief, a unique sample adapter was applied to each patient sample, followed by an overnight incubation at 16° C. After ligation, library molecules underwent amplification, then the quantity and size of pre-capture libraries were validated using the Qubit dsDNA High Sensitivity Assay (ThermoFisher) and 2100 Bioanalyzer (Agilent Technologies) with the High Sensitivity DNA Kit. Subsequently, library enrichment was performed using the AVENIO ctDNA enrichment kit, and sequence capture was carried out using the AVENIO surveillance panel and the AVENIO post-hybridization sub-kits following the manufacturer's instructions. Enriched samples then underwent a second PCR amplification step in preparation for sequencing. Final library concentrations were determined using the Qubit dsDNA High Sensitivity Assay, and library fragment size distributions were determined using the High Sensitivity DNA kit on Bioanalyzer. The final libraries were sequenced using 2×150 bp paired-end reads to target approximately 40M reads per sample on a NovaSeq S4 instrument (Illumina).

Sequencing data were analyzed using the AVENIO Oncology Analysis Software, which performs quality control checks and identifies single nucleotide variants (SNVs) along with their allele frequencies (AFs) for each sample. All analyzed samples passed quality control assessments. For each non-synonymous SNV, we calculated its maximum AF across all 62 samples. SNVs with a maximum AF >20% were excluded as potential germline variants, with exceptions made for variants in genes harboring at least one non-synonymous mutation in over 10% of TCGA melanoma samples⁶. The ctDNA amount was quantified as the mean AF of all remaining non-synonymous SNVs in each sample.

### Data Collection and Processing

scRNA-seq Collection and Processing

Single-cell RNA-seq atlases of melanomas and carcinomas, either newly generated in this work or obtained from published studies as pre-processed data, were annotated as B cells, plasma cells, CD4 T cells, CD8 T cells, NK cells, macrophages, dendritic cells, endothelial cells, fibroblasts, and non-immune/non-stromal cells. For publicly available datasets with author-supplied annotations (breast cancer, colon cancer, liver cancer, squamous cell carcinoma, and melanoma), annotations were mapped to the above cell type labels. Cohort 1 scRNA-seq data, as well as publicly available data without cell type annotations (bladder cancer, lung cancer, ovarian cancer, prostate cancer, and pancreatic cancer), were analyzed using Seurat (v4.3.0) as described below. For quality control, cells with fewer than 200 detected genes or more than 25% of reads mapped to mitochondrial genes were excluded. Raw counts were imported and cells clustered following SCTransform with the glmGamPoi method, FindVariableFeatures, ScaleData, RunPCA, FindNeighbors, and FindClusters. Cell type annotations were then manually assigned to clusters based on the average expression of canonical lineage markers: PECAM1 and VWF for endothelial cells, COL1A1 and COL3A1 for fibroblasts, CD8A and CD8B for CD8 T cells, CD4 and IL7R for CD4 T cells, GNLY and NCAM1 for NK cells, CD68 and CD14 for monocytes/macrophages, CD1C for dendritic cells, IGKC and MZB1 for plasma cells, CD79A for B cells, and EPCAM for epithelium and SOX9, MET, and MYC for melanoma. Clusters expressing multi-lineage markers were considered potential doublets/multiplets and eliminated from further analysis.

### MERSCOPE Collection and Processing

Preprocessed MERSCOPE profiles of 15 formalin-fixed paraffin-embedded (FFPE) human tumor specimens, spanning melanoma and six distinct carcinomas, were downloaded from Vizgen (MERSCOPE FFPE Human Immuno-oncology program). Three ovarian cancer samples were excluded due to significant tissue fragmentation. For quality control, genes expressed in fewer than 5 cells and cells with fewer than 300 total transcripts were excluded from each remaining sample.

For cell type annotation, transcripts were downsampled to 300 per cell, and cells were clustered with Seurat (v4.3.0) using the following steps: NormalizeData, FindVariableFeatures (nfeatures=300), ScaleData, RunPCA, FindNeighbors, and FindClusters (resolution=1). Cell type annotations were then assigned as described for “scRNA-seq collection and processing” above, with re-clustering of individual clusters, particularly those containing mixed lymphocyte groups, performed for additional resolution as needed.

To remove ambient and improperly segmented mRNAs, publicly available tumor scRNA-seq atlases (“scRNA-seq collection and processing” above) were used to identify genes commonly expressed in each cell lineage. Specifically, for each cell type, genes expressed in at least 5% of cells in three or more cancer types were identified, resulting in a whitelist for each cell type. Genes in each cell type that were absent from the corresponding whitelist were then set to zero expression in the MERSCOPE data. Subsequently, cells with fewer than five detectably expressed genes were excluded, resulting in a final dataset of 5.6M evaluable cells from 12 samples.

### CosMx Spatial Molecular Imager (SMI) Data Collection and Processing

The Seurat object for the FFPE human liver sample was obtained from NanoString. Cells from the hepatocellular carcinoma samples were selected and grouped into the previously enumerated cell types (“scRNA-seq collection and processing”) based on the available cell type annotations in the Seurat object.

### Visium/Legacy Spatial Transcriptomics (ST) Data Collection and Processing

Processed data from 39 Visium and 51 legacy ST profiles of carcinoma and melanoma samples were downloaded from 10× Genomics and 12 previous studies. For quality control, genes expressed in fewer than 5 spots and spots expressing fewer than 200 unique genes were omitted.

Integration of Spatial Transcriptomics and scRNA Data with CytoSPACE

CytoSPACE (v.1.0.3) was employed to align single-cell transcriptomes from scRNA-seq data to the ST data from the same cancer type, reconstructing whole-transcriptome single-cell spatial expression profiles. The analysis was performed separately for each ST sample. To eliminate potential bias derived from different total UMIs, raw counts from droplet-based scRNA-seq data were downsampled to 1,500 total UMIs per cell, while TPM data from Smart-seq2 data (for melanoma) were used as is. The lap_CSPR solver and recommended settings were applied for all analyses, including the default mode for bulk ST (Visium and legacy ST), single-cell mode for single-cell ST data (MERSCOPE and CosMx SMI), and an average of five cells per spot for Visium data and 20 cells per spot for legacy ST data.

### Tumor and Stroma Region Annotation

For all ST data, sample regions were partitioned into tumor and adjacent stromal regions for subsequent analyses.

### Single-Cell ST Data

A previously established density-based methodology was employed to annotate tumor and adjacent stromal regions in single-cell ST data (MERSCOPE and CosMx SMI). The approach involves overlaying a 200 μm×200 μm grid onto the image and subsequently computing the fraction of non-immune/non-stromal cells within each grid. The non-immune/non-stromal fractions across all grids exhibit a bimodal distribution, with one peak denoting grids in the tumor region and the other denoting grids in the adjacent normal region (data not shown). A threshold was then determined to categorize grids with a high abundance of non-immune/non-stromal cells as tumor regions and the others as adjacent normal regions. In cases where clear peaks were absent, a smaller grid size (e.g., 100 μm×100 μm) was used to achieve the bimodal distribution. To further refine the annotations, a secondary analysis was conducted. For each single cell, the neighboring cancer cells within a 100 μm radius were counted. Cells with more than five neighboring cancer cells were grouped into tumor regions, while the others were designated as adjacent normal regions.

### Bulk ST Data

A machine learning approach was implemented to annotate tumor and adjacent stromal regions in bulk ST data, including Visium and legacy ST data. For model development, eight Visium samples and 21 legacy ST samples from four cancer types with available pathologist annotations were used. We categorized all sample regions as either tumor or adjacent stromal, then trained a logistic regression model to classify regions based on four features: the number of detectably expressed genes in each spot, the abundance of non-immune/non-stromal cells in each spot, the average number of expressed genes in one-hop neighboring spots, and the average abundance of non-immune/non-stromal cells in one-hop neighboring spots. Abundances of non-immune/non-stromal cells were inferred using spacexr (v2.0.0) in full doublet mode²⁸, with raw counts from ST and scRNA-seq data as input. To eliminate sample and platform differences, the four features were normalized to unit variance within each sample. To ensure balanced representation across ST platforms and cancer types for training, the spots were downsampled within each cancer type across ST platforms and then across cancer types. The final model trained on all samples was used to predict tumor and adjacent stroma regions in all other Visium and legacy ST samples lacking pathologist annotations.

### Validation of Bulk Region Annotation

The performance of the model was evaluated using leave-one-cancer-type-out cross-validation (LOOCV). In each iteration, a model was trained on samples from three cancer types, then applied to samples of the held-out cancer type. The consistency between annotated and predicted regions was quantified by area under the receiver operating characteristic curve (AUC).

We additionally performed differential expression analysis between tumor and adjacent stromal spots using both annotated and predicted region annotations. Mean logarithm fold changes (LFC) of genes were computed within each sample by comparing tumor and adjacent stromal spots. The gene LFCs were then averaged across samples within each cancer type and subsequently across the four cancer types. The concordance of the differential gene expression identified by annotated and predicted regions was quantified using Pearson correlation.

### Distance to Tumor Margin

We assessed the distance of each single cell or spot to the tumor/stromal interface by computing the shortest Euclidean distance. A positive distance was used for cells/spots localized to the tumor region, and a negative distance was used for cells/spots localized to the adjacent stromal region.

### Differential Expression in Tumor and Stroma

We analyzed scRNA-seq data mapped to ST samples (see “Integration of spatial transcriptomics and scRNA data with CytoSPACE”), with differential expression analysis performed independently for nine TME cell types. As validation, a similar analysis was conducted using MERSCOPE data for CD8 T cells, CD4 T cells, and macrophages. Prior to these analyses, data normalization was tailored for each platform. UMI-based scRNA-seq data were normalized for each cell type separately using SCTransform from Seurat (v4.3.0); Smart-seq2 data from melanoma samples were normalized to log₂ TPM; and MERSCOPE data were normalized with NormalizeData from Seurat (v4.3.0).

### Pan-Cancer Differentially Expressed Genes

To identify differentially expressed genes conserved across cancer types, Visium datasets were selected due to the breadth of malignancies previously covered on the platform, including specimens from melanomas and the nine carcinoma types evaluated in this work. Differential expression between tumor and adjacent stromal regions was quantified using FoldChange from Seurat (v4.3.0), for each cell type and each sample separately. Gene-level LFCs were then averaged across replicates, across all samples within each cancer type, and across all cancer types in the discovery datasets. The top 100 HUGO protein-coding genes (HGNC) with the highest LFC in tumor or adjacent stromal regions were selected and then validated across held-out samples, including legacy ST, MERSCOPE, and CosMx SMI.

Separately, pan-cell-type conserved differentially expressed genes were identified by computing the average LFC across all cell types using the discovery datasets. The top 100 HUGO protein-coding genes with the highest average LFC across all cell types were selected and validated across different cell types and ST platforms, including legacy ST, MERSCOPE, and CosMx SMI.

### Enrichment of Known Cell States

A set of 135 unique cell states and their markers were curated from seven studies focused on the single-cell tumor microenvironment. To test the enrichment of these cell states in tumor or adjacent stromal compartments, fgsea (v1.25.1) was run independently for each evaluable cell type, with the top 50 markers for each cell state and average LFC across all cancer types in the discovery datasets as input.

Additionally, fgsea (v1.25.1) was performed to test the enrichment of pan-cell-type differentially expressed genes in MsigDB hallmark pathways, using the average LFC across all cell types in the discovery datasets as input.

### Spatial EcoTyper Framework

Despite experimental advances enabling high resolution expression profiling of cells in situ, leveraging such data to systematically profile the co-association of cell states into spatial ecotypes (SEs) and discover conserved SEs across different specimens and cancer types has remained challenging. The spatial organization of cell states and their relative abundances within an ecotype can differ significantly across regions and between samples, and even expression profiles of cells sharing the same phenotypic cell state exhibit a range of natural variability. In addition to high biological heterogeneity, technical dropout, sample-specific batch effects, and platform-based batch effects all pose obstacles to SE discovery.

With these considerations in mind, we developed Spatial EcoTyper. At its core, the framework relies upon a network integration technique to identify common patterns of spatial transcriptomic variation shared across samples. This is achieved by adapting similarity network fusion (SNF), a previously described approach for multi-omics data integration across patients, which is inherently robust to batch effects. By introducing a series of carefully constructed spatial gene expression profiles, our approach mitigates technical dropout while providing stability under biological variation.

The Spatial EcoTyper framework consists of five components, described in detail in the following sections.

Determination of sample-level spatial clusters: In each single-cell ST sample, spatial expression data is encoded into cell-type-specific profiles of microregions, spatial covariation among the microregions is computed via SNF, and microregions are clustered over the resulting network.

Identification of conserved spatial ecotypes: Spatial clusters discovered from individual single-cell ST samples are represented by gene expression profiles of their associated cell states, and clusters with highly covarying cell states are clustered into conserved SEs across samples.

Discovery of conserved SE-specific cell states: Cell states uniquely enriched in each SE and conserved across samples are identified using a specialized variant of non-negative matrix factorization (NMF).

Recovery of conserved SE-specific cell states: An NMF model is developed to recover SE-specific cell states in external single-cell or spatial expression datasets.

Deconvolution of SEs from bulk RNA-seq: The approach from the previous component is generalized to the task of recovering SE abundances from bulk RNA-seq, using a training cohort of pseudo-bulk mixtures.

### Determination of Sample-Level Spatial Clusters

The Spatial EcoTyper framework begins by identifying clusters of microregions within each single-cell ST sample. While ST data should be generated by the same assay, the discovery phase is applicable to diverse single-cell ST platforms, including Vizgen MERSCOPE, NanoString CosMx, and 10× Xenium. For this study, we analyzed tumor samples profiled using MERSCOPE, with normalization performed as described in the “Spatial EcoTyper discovery cohort” below.

### Assembly of Cell-Type-Specific Microregion Gene Expression Profiles

As a first step, the framework encodes spatial expression data into cell-type-specific microregion gene expression profiles (cmGEPs), in which gene expression profiles (GEPs) per cell type are prepared for spatial microregions centered along a regular grid. In detail, for each microregion, a cmGEP for each cell type was constructed by averaging the normalized GEPs of the nearest up to k cells of the given cell type located within radius r of the microregion center (selected to be 50 μm in practice; see “Microregion radius” below). For each cell type c, cmGEP vectors of all microregions were then concatenated into a cmGEP matrix E^{c} of dimension g genes by m microregions. To identify SEs containing multiple cell types, microregions characterized by a single cell type were eliminated from our analyses. Additionally, from each E^{c}, genes expressed in fewer than five microregions, microregions with no cells of type c, and microregions expressing fewer than five genes were excluded.

The cmGEP serves as a fundamental data unit for Spatial EcoTyper. It mitigates technical dropout in single-cell gene expression profiling by aggregation over multiple cells while simultaneously providing a representation that mitigates the influence of cell type abundance. Hence, the cmGEP representation is suitable to ecotype detection based on cell state variation, rather than shifts in local cell type composition alone.

### Microregion Similarity Network Construction

After preparing the cmGEP matrix E^{c}, the next step is to construct a similarity network of microregions for each cell type c; in other words, to prepare a set of c pairwise-similarity matrices A^{c}, each of dimension m×m, describing the similarity of cmGEPs between microregions. To do so, we first performed dimension reduction on each matrix E^{c} to identify the top 20 principal components using the RunPCA function from the Seurat (v4.3.0). Pairwise similarities between cmGEPs were then calculated as inverted Euclidean distance, forming the similarity matrix A^{c}. Given the typically large number of microregions, we retained only the top a highest similarities in each row and column of A^{c}, setting all other values to zero to create a sparse matrix. While α=50 was used in this work, we note that a was robust to a range of empirically tested values (data not shown). This step maintains key edges in the network and enhances scalability. For any instance in which the given cell type c was not represented in both microregions, the corresponding entry in A^{c} was assigned as NA.

### Microregion Similarity Network Fusion

Following similarity matrix construction, the framework proceeds by fusing all A^{c} matrices across cell types into a single similarity matrix A using SNF. This resulting matrix encodes spatial community structure by linking microregions exhibiting high covariation across cell types; in other words, by connecting microregions in which the presence of a certain phenotypic state of one cell type corresponds to the presence of certain phenotypic states among other cell types.

To achieve this network fusion in practice, we implemented an enhanced version of the SNF function from the SNFtool R package (v2.3.1), adding support for sparse matrices and missing values while otherwise preserving the original functionality, then applied this updated function to our set of A^{c} matrices. We then performed a rank normalization over the columns of the resulting matrix to transform similarity values per column into a standard space. We then converted ranked values per column to zero minimum and unit maximum.

### Spatial Clustering and Cluster Profiling

Given the fused similarity matrix A, the Spatial EcoTyper framework groups spatial microregions into clusters, which will become candidates for SEs when considered across multiple samples as described in the following section (“Identification of conserved spatial ecotypes”). To perform clustering of microregions, we used a standard Seurat clustering pipeline, sequentially applying RunPCA, FindNeighbors and FindClusters functions. For single-sample analyses, the resulting spatial clusters represent sample-level SEs. For integrative analysis across samples, a higher clustering resolution is recommended to enhance robustness to parameter variation (see “Clustering resolution”).

In practice, microregions within pre-annotated domains can be balanced to ensure equal representation before clustering. To obtain an equal number of microregions from tumor and adjacent stromal regions in this work, we uniformly downsampled the one with more microregions (e.g., tumor) prior to integrative analyses.

### Identification of Conserved Spatial Ecotypes

Beyond sample-level SE analysis, a key strength of the Spatial EcoTyper framework lies in its ability to identify SEs conserved across a variety of conditions-such as across samples, patients, and cancer types. To identify such SEs, Spatial EcoTyper utilizes a variant of the sample-level process described above (“Determination of sample-level spatial clusters”), using spatial clusters rather than microregions as the fundamental units.

### Assembly of Sample Cell-Type-Specific Spatial Cluster Gene Expression Profiles

Following clustering of microregions within each sample (as described in “Spatial clustering and cluster profiling”), Spatial EcoTyper computes cell-type-specific gene expression profiles for each microregion cluster, referred to as ccGEPs, representing the average gene expression profile per cell type within each spatial cluster. Each cell was assigned to the cluster of its spatially nearest microregion for ccGEP computation. To minimize batch effects across samples, row-based standardization was applied to each single-cell GEP, normalizing gene expression to zero mean and unit variance per gene before ccGEP computation, facilitating the integration across samples. Akin to the cmGEPs, ccGEPs enable robust characterization of cell states within each spatial cluster, independent of relative abundances of parent cell types.

Sample-level ccGEPs were subsequently aggregated per cell type across samples into a matrix E′^{c}, each of dimension g genes by s sample-level spatial clusters, with genes here restricted to those with nonzero expression in at least some microregion in each sample. In practice, to ensure sufficient representation and well-defined computations per sample, we require a minimum of three spatial clusters containing the cell type c for inclusion of a sample into each E′^{c}. Next, the feature space of each E′^{c} was reduced to the top 200 variable genes, where highly variable genes per matrix were computed according to their rank product of variances across samples. In other words, for each cell type, the variance of each gene across spatial cluster ccGEPs was computed per sample, with genes then assigned a rank by variance. Ranks were then aggregated across samples by geometric mean, and the top highly variable genes were selected per E′^{c} from the result.

### Cross-Sample Spatial Cluster Similarity Network Construction

From this updated representation, a similarity network of spatial clusters was constructed per cell type. These were computed via Spearman correlation of the columns of E′^{c} for each cell type, yielding a set of c pairwise similarity matrices A′^{c} describing the similarity of ccGEPs between sample-level spatial clusters. For any pairwise comparison of spatial clusters in which cell type c was not represented in both spatial clusters, the corresponding entry in A′^{c} was assigned as NA. To minimize any remaining batch effects, we standardized the similarity matrices A′ by performing rank normalization independently on each submatrix

$${A}_{ij}^{′ c} ,$$

which represents the similarities of spatial clusters between sample i and j. The normalization was performed by converting the non-NA entries of each column in

$${A}_{ij}^{′ c}$$

to ranks and rescaling the ranks to the unit interval.

### Cross-Sample Spatial Cluster Similarity Network Fusion

The Spatial EcoTyper framework proceeds by fusing all A′^{c} matrices across cell types into a single similarity matrix A′ using the enhanced SNF function as described above in “Microregion similarity network fusion.” The resulting matrix encodes the conservation of spatial community structures by linking sample-level spatial clusters with high covariation across cell types.

Clustering of Sample-Level Spatial Clusters into Spatial Ecotypes

To robustly group sample-level spatial clusters into SEs, NMF clustering was applied to A′, with the number of clusters, i.e. rank, set according to the following procedure. NMF clustering of A′ was tested for ranks ranging from 2 to 50, with 50 runs per rank using the Brunet method³⁹, with optimal rank then selected as the highest rank for which the cophenetic coefficient exceeded 0.95 and subsequently showed the greatest drop. The NMF results derived from the selected rank, here identified as 11, were used to group sample-level spatial clusters and corresponding microregions and single cells into SEs.

### Assembly of Cell-Type-Specific SE Gene Expression Profiles

Having assigned single cells to SEs, Spatial EcoTyper can then determine SE cell state gene expression profiles (csGEPs). For each cell type, NMF was performed on single-cell GEP E*^{c} and SE label matrix H^{c}, to construct csGEPs. Here, the E*^{c} represents a gene-by-cell matrix for each cell type c, for which an equal number of cells (at least 300 cells, and up to 5,000 cells) were randomly selected from each sample-SE pair to ensure balanced representation. Normalized GEPs of selected cells were standardized to zero mean and unit variance per gene within each sample and then concatenated into the csGEP matrix E*^{c}. The SE label matrix H^{c} is a binary cell by SE matrix indicating SE membership for each cell in E*^{c}. NMF was applied to solve the equation:

$${E}^{* c} = {W}^{c} \times {H}^{c}$$

where W^{c} represents the basis matrix containing csGEPs, which summarize the GEP for SE-specific cell states of each cell type c. To refine the csGEPs for cell state recovery, the top 50 genes were chosen per SE based on the largest positive delta compared to the second highest expression across SEs. Each basis matrix was then reduced to the selected genes. These refined basis matrices enable the recovery of SEs and their cell states from data outside of the discovery cohort using the NMF framework (see “Recovery of SE-specific cell states”).

### Discovery of Conserved SE-Specific Cell States

While SEs are derived from spatial covariation in cell states across cell types, shared across samples, not every cell state associated with an SE need be specific to that SE. To identify and validate cell states specifically enriched in each SE and conserved across discovery samples, we repeated the process of csGEP identification as described above (“Assembly of cell-type-specific SE gene expression profiles”) over subsets of discovery cohort samples, using the resulting NMF basis matrices to predict cell state labels on held-out data in a leave-one-sample-out cross-validation (LOOCV) process. For label assignment, NMF prediction output matrices H^{c} were standardized to unit sum per column, yielding a probability matrix denoting the probability of each single cell being localized in each SE. Cells were then assigned to the SE-associated cell state with the highest probability. For each LOOCV iteration, the enrichment of each cell state within each SE was assessed by its ability to correctly assign cells to that SE, measured by the F1 score.

To ensure robustness, this LOOCV process was repeated 20 times, with F1 scores averaged across all repetitions. Cell states were considered specific to an SE only if the associated F1 score exceeded the second highest F1 score for the cell state across other SEs by at least 0.1. Otherwise, the cell state was deemed either broadly distributed across multiple SEs or not conserved across samples. Using this approach, 38 SE-specific cell states were identified, specific to nine SEs. Two SEs identified initially were excluded from further analysis due to a lack of specific cell states, and the remaining SEs were renumbered accordingly from 1 to 9 based on their average distance across discovery samples to the tumor/stromal margin.

### Recovery of SE-Specific Cell States

After identifying conserved SE-specific cell states through the above LOOCV process, we used all discovery cohort samples to prepare an ensemble basis matrix W*^{c} for each cell type. To do so, we repeated the process described in “Assembly of cell-type-specific SE gene expression profiles” 50 times, including only the selected SE-specific cell states and averaging the resulting basis matrices per cell type to produce W*^{c}. For feature selection, genes showing the highest and most specific expression in each cell state were identified from each basis matrix as previously described, and then genes that were selected for the same cell state in over half of the repetitions were retained in the ensemble matrix.

The resulting ensemble matrices comprise a core component of the Spatial EcoTyper framework and can be used to recover SE-specific cell states from external single-cell transcriptomics datasets using NMF as described previously. To ensure robust predictions, we restrict cell state assignments to cases in which the prediction probability exceeds 0.6, otherwise designating cells as belonging to a null class, referred to as ‘NonSE’.

Deconvolution of SEs from Bulk RNA-seq

To enable profiling of SEs in bulk RNA-seq data, the Spatial EcoTyper framework includes an NMF model trained over simulated bulk RNA-seq prepared by aggregation of scRNA-seq data into pseudo-bulk mixtures for which ground truth SE proportions are known.

### Construction of Pseudo-Bulk Mixtures

Previously described publicly available scRNA-seq data (see “scRNA-seq data collection and processing”) from 10 cancer types were used to create pseudo-bulk mixtures. First, cells were annotated as described in “Recovery of cell states and SEs from scRNA-seq,” labeled according to the parent SE of their assigned cell state or, if unassigned, designated ‘NonSE’, for a total of 10 label classes. The fractional composition of each pseudo-bulk was generated by random sampling of a value per label class from the Gaussian distribution N(μ=2, σ=1), with negative values set to zero and the resulting values normalized to unit sum.

Pseudo-bulks were assembled separately by cancer type, with GEPs constructed by aggregating 1,000 cells randomly selected to satisfy the predefined fractions of cell states. For UMI- and plate-seq-based data, raw counts and TPM values were respectively summed across selected cells. One hundred pseudo-bulks were generated per cancer type, and the resulting GEPs were normalized using the NormalizeData function from Seurat (v4.3.0). To mitigate cancer type and batch differences, GEPs were further normalized to zero mean and unit variance per gene within each cancer type.

### NMF Model Training for Bulk Deconvolution

The resulting profiles were concatenated into gene by sample matrix E^{P}, with genes limited to those detected across all cancer types, and pseudo-bulk SE fractional abundances were encoded in sample by label matrix H^{P}. From these, a basis matrix was derived by application of NMF followed by feature selection as described previously (“Assembly of cell-type-specific SE gene expression profiles”). The resulting basis matrix W^{B} constitutes another core component of the Spatial EcoTyper framework and can be used to deconvolve SE fractional abundances from any bulk gene expression data. Bulk sample predictions are performed via NMF as described in “Discovery of conserved SE-specific cell states,” excluding the final classification step to yield fractions instead. The resulting matrix H^{P} includes the abundance of SEs across input bulk samples. In practice, to perform deconvolution, input data should be normalized to CPM or TPM as appropriate, log₂-adjusted, and then normalized across samples to zero mean and unit variance per gene.

### Spatial EcoTyper Discovery Cohort

MERSCOPE samples (see “MERSCOPE collection and processing”) were selected for SE discovery owing to their high spatial resolution and the availability of samples across multiple tumor types. To capture spatial microenvironments from both tumor and adjacent stromal regions, we selected nine samples with more than 5% of cells in the latter. These samples cover six cancer types, including two melanoma, two colon cancer, two liver cancer, one breast cancer, one prostate cancer, and one ovarian cancer sample(s). Five of these samples, each from a different cancer type, were then used for SE discovery. Prior to analysis, MERSCOPE samples were preprocessed as described in “MERSCOPE collection and processing”, then standardized using the NormalizeData function from the Seurat (v4.3.0). We then focused on nine major TME cell types for SE discovery and characterization: B cells, plasma cells, macrophages, DCs, CD4 T cells, CD8 T cells, NK cells, fibroblasts, and endothelial cells. Malignant cells were not included owing to significant differences across tumor types.

### Selection of Spatial EcoTyper Parameters

### Microregion Radius

When applying Spatial EcoTyper to individual samples (“Determination of sample-level spatial clusters”), we consistently observed a phenotypic spatial continuum resembling the physical distance of microregions to tumor margin. To assess robustness, Spatial EcoTyper analyses were performed with 16 different microregion radii, ranging from 20 μm to 300 μm, on a melanoma sample (‘Melanoma 1’). For each microregion radius, the correlation between the phenotypic continuum and physical distance of microregions to the tumor margin was evaluated. To do this, a “pseudospace” trajectory of microregions was inferred from each UMAP embedding using Slingshot⁴¹, an approach for inferring cell lineage and pseudotime from scRNA-seq data. Specifically, microregions were grouped into two clusters using k-means clustering on the top 10 PCs, and then a one-dimensional “pseudospace” trajectory was inferred using the getLineages and getCurves functions (using the parameters approx_points=300 and allow.breaks=FALSE) from the Slingshot R package (v2.4.0). The correlation between the pseudospace of microregions and their physical distance to the tumor margin was quantified with Spearman correlation, computed with the weightedCorr function from the wCorr R package (v1.9.8), with tumor and adjacent stromal regions weighted equally. A radius of 50 μm was selected for SE discovery, as it provided high granularity and good linear correlation between the inferred pseudospace and the physical distance of microregions to the tumor margin.

### Clustering Resolution

A key parameter in the Spatial EcoTyper integrative analysis is the resolution for Louvain clustering, which groups microregions into clusters within each sample. To ensure robustness, 11 different resolutions ranging from 1 to 50 were tested, and all resulting spatial clusters were grouped into ten SEs. The similarity between SEs derived at different resolutions was evaluated using the average Adjusted Rand index (ARI), comparing each resolution with the others. The discovered SEs show high overall consistency, with results being more stable when a resolution higher than 15 was used. The resolution of 30, which had the highest average ARI, was selected for SE discovery in our analysis.

### Recovery of Cell States and SEs for Validation Cohort

### MERSCOPE

Using the previously described approach (“Recovery of SE-specific cell states”), SE-specific cell states were recovered from five discovery samples with LOOCV and four held-out MERSCOPE samples. In this process, all single cells were classified as SE-specific cell states or NonSE, which allowed for further grouping of single cells into respective SEs.

### Bulk ST

For SE validation, 26 Visium and 70 legacy ST samples were selected, each containing at least five spots located over 500 μm away from the tumor margin. Spatial GEPs were normalized to zero mean and unit variance per gene across all spots. Using the cell state recovery models (see “Recovery of SE-specific cell states”), we calculated an H*^{c} matrix for each cell type c, representing cell state abundances across spots. The relative SE abundances for each SE were then determined by averaging the abundances of the cell states associated with each SE. Lastly, each spot was assigned to the SE with the highest abundance.

scRNA-seq Data

To validate SEs, we also used scRNA-seq data (see “scRNA-seq data collection and processing”) from 135 tumor samples across 10 cancer types. For each cell type from each carcinoma type, the scRNA-seq data were normalized using the SCTransform from Seurat (v4.3.0), while log₂ TPM data were used for melanoma as the data were from Smart-seq2. Cell state recovery was performed as previously described (“Recovery of SE-specific cell states”), and the abundance of a cell state (or SE) in a sample then determined as the fraction of cells assigned to that cell state (or SE) out of the total cells in the sample.

### Validation of SEs

Two experiments were conducted to validate SEs. The first experiment examined the distance of SEs to the tumor margin using ST data. Distances of SEs to the tumor margin were determined after SE recovery and compared between discovery and validation datasets. The second experiment tested the spatial co-localization and co-association of SE-specific cell states using both ST and scRNA-seq data. For spatial co-localization, cell states were recovered from single-cell ST data, and their spatial co-localization was evaluated within each sample. For co-association, cell states were recovered from Visium and legacy ST data, and the correlation between cell state abundances across spots was assessed. Additionally, cell states were recovered from scRNA-seq data, the abundance of each cell state within each sample was determined, and then the co-association of cell states was evaluated by correlating their abundances across different tumors.

### Distance of SEs to Tumor Margin

Following SE recovery from ST data, the distance of each SE to the tumor margin was computed by averaging the distance of microregions or spots assigned to the SE within each sample. These distances were further averaged across all discovery or validation samples. The distance of each SE to the tumor margin in the discovery samples was computed in the same manner, using the SE grouping from the discovery. The consistency between distances derived from discovery and recovery grouping was evaluated using Pearson correlation.

### Cell State Co-Localization

The spatial co-localization patterns of SE-specific cell states were validated using nine MERSCOPE samples, with cell states recovered from each sample. For each single cell, the fractional abundances of neighboring cell states within a 50 μm radius were determined, resulting in an N×S matrix, F, where Fᵢⱼ denotes the fraction of cells within a 50 μm radius of cell Nᵢ that are of state Sⱼ. Single-cell level fractions were subsequently averaged within each cell state, producing an S×S co-localization matrix, L, where Lᵢⱼ represents the average fractional abundance of cell state Sᵢ near cell state Sⱼ. To control for biases, permutation experiments were performed. Cell state assignments were shuffled 10,000 times, and co-localization matrices were recomputed, yielding 10,000 random co-localization matrices, L^{rand}. The co-localization matrix L was then normalized by subtracting the average of the L^{rand} matrices and dividing by their standard deviation for each element:

$${L}_{ij}^{′} = \frac{{L}_{ij} - {μ}_{{L}_{ij}^{rand}}}{{σ}_{{L}_{ij}^{rand}}}$$

This resulted in a matrix L′, where Lᵢⱼ′ represents co-localization index between cell state Sᵢ and cell state Sⱼ. Lastly, the co-localization indexes from multiple samples in each dataset were integrated using Stouffer's approach.

### Cell State Co-Association

Cell state co-associations were evaluated in 26 Visium, 70 legacy ST data, and scRNA-seq data from 135 tumors. Specifically, cell state abundances were determined for each spot (bulk ST data) or sample (scRNA-seq data) as described in “Recovery of cell states and SEs for validation cohort” above. Co-associations were then assessed by computing Pearson correlations of cell state abundances across spots (bulk ST) or samples (scRNA-seq). The co-association scores for each pair of cell states were averaged across samples within each cancer type (bulk ST data) and then across cancer types (both bulk ST and scRNA-seq data).

### Significance of Cell State Co-Localization and Co-Association

Permutation experiments were performed to assess the significance of cell state co-localization or co-association, collectively referred to as co-occurrence hereafter. For each cell state Sᵢ, the average co-occurrence score with other cell states in the same SE were computed. This score was compared with 10,000 corresponding scores obtained by randomly shuffling the co-occurrence scores between cell state Sᵢ and all other cell states. A p-value was calculated to quantify the probability of cell state Sᵢ having a higher co-occurrence with cell states within the same SE, using the formula:

$${P}_{i} = \frac{{\sum }_{j = 1}^{10 , 000} I ( {L}_{i} > {L}_{i}^{( j )} )}{10 , 000}$$

where Lᵢ represents the actual average co-occurrence score of cell state Sᵢ and

$${L}_{i}^{( j )}$$

represents the average score of cell state Sᵢ from the j-th permutation. I

$$( {L}_{i} > {L}_{i}^{( j )} )$$

is an indicator denoting that Lᵢ is higher than

$${L}_{i}^{( j )} .$$

Stouffer's method was applied to aggregate the p-values of cell states for each SE, resulting in a meta p-value indicating the significance of cell state co-occurrence within the SE.

### Validation of Bulk Deconvolution

### Cross Validation Over Training Pseudo-Bulks

LOOCV was conducted to evaluate the performance of the SE deconvolution model. This process involved training the NMF model (see “NMF model training for bulk deconvolution”) on pseudo-bulk GEPs from nine cancer types and then using the trained model to deconvolve SEs from the pseudo-bulk GEPs of the held-out cancer type (see “Construction of pseudo-bulk mixtures”). The consistency between predicted SE levels and the ground truth SE abundances was then assessed by computing the Pearson correlation across 1,000 pseudo-bulk mixtures (FIG. 4a,c and Extended Data FIG. 8a,b).

### Performance Evaluation of SE Deconvolution Model

We further evaluated the performance of the SE deconvolution model using paired scRNA-seq and bulk RNA-seq data from Cohort 1. First, SEs and their cell states were recovered from scRNA-seq data (see “Recovery of cell states and SEs for validation cohort”), and the SE abundances in each sample were determined. Next, SE abundances were inferred from the bulk RNA-seq using the SE deconvolution model (see “NMF model training for bulk deconvolution”). Due to the limited number of samples, which could bias the centering and unit variance normalization required for SE deconvolution, we combined Cohort 1 and TCGA RNA-seq data from matched cancer types, removed batch effects between the two datasets using the Combat function from the sva R package (v3.46.0) with default parameters, and then normalized the gene expression to zero mean and unit variance per gene across all samples. This process was conducted for melanoma and colon cancer separately. The resulting data were then used to infer SE abundances. Finally, the consistency of SE abundances inferred from scRNA-seq and bulk RNA-seq data was assessed by computing Pearson correlation.

### Performance Assessment and Benchmarking of Spatial EcoTyper

To systematically benchmark Spatial EcoTyper against related methods, we established a framework to score spatial ecotype clusters based on four key criteria: (i) the diversity of cell types per cluster; (ii) the degree of spatial co-localization among member cells of each cluster; (iii) the specificity of cell-type GEPs for each cluster; and (iv) the ability to identify conserved spatial ecotype clusters across ST samples following integration. We then conducted two experiments: one focusing on (i), (ii), and (iii), assessing spatial ecotype discovery within individual samples, and the other targeting (i) and (iv), evaluating spatial ecotype discovery across samples.

### Metrics

The following four metrics were employed to measure the abovementioned criteria:

Cell type mixing. This metric, which evaluates the diversity of cell types within each cluster, is computed as n/9, where n is the number of cell types (of nine TME cell types analyzed in the study) within a given cluster.

Spatial co-localization. This metric evaluates the degree to which cells of a given cluster show spatial co-localization. To promote a fair comparison, cells were first partitioned into the same grid of non-overlapping microregions used for Spatial EcoTyper, each with a radius of 50 μm (see “Clustering of sample-level spatial clusters into spatial ecotypes”). Next, each microregion was labeled by the dominant spatial cluster within it (i.e., the cluster encompassing the most cells). Then, for each microregion i labeled as spatial cluster j, the fraction of neighboring microregions labeled as cluster j and located within a 100 μm radius from the center of microregion i was calculated. The resulting fractions were then averaged across microregions within each cluster and subsequently across all clusters to yield an overall score. Consistent results were also observed for radii of 150 μm and 200 μm (data not shown).

Mean silhouette width. This metric evaluates, for each cell type, the degree of GEP separation between clusters and the consistency of GEPs within them. For consistency across methods, n meta-cells were generated by averaging log₂ GEPs of randomly selected cells (10 cells per meta-cell, with no overlap) within each spatial cluster. Using the resulting meta-cell GEPs, the silhouette width was calculated for each cell type, then averaged across cell types. Analyses using n=10, 20, 30, and 50 meta-cells yielded consistent results (data not shown).

Sample mixing. This metric measures the diversity of samples within each cluster, reflecting each method's ability to integrate data across samples. It is computed as (1−f)×2, where f is the fractional representation of the dominant sample. A higher score indicates better integration while a score of 0 indicates failed integration.

### Methods

The performance of Spatial EcoTyper was benchmarked against seven published methods—CellCharter, SPACE, SEDR, BANKSY, SpatialPCA, STAGATE, and UTAG—for identifying multicellular communities from MERSCOPE data. SpaceFlow⁵¹ was initially considered but excluded from the final benchmarking due to unresolved issues with its tutorial. Unless otherwise noted, all methods were evaluated using their default settings.

CellCharter. CellCharter (v0.3.1) applies a published variational autoencoder, scVI, for dimension reduction and batch effect correction. It clusters cells using a Gaussian mixture model, based on aggregated features from each cell and its 1-hop, 2-hop, and up to n-hop neighbors, with n=3 used in our analysis as recommended in the tutorial.

SPACE. SPACE (v0.7.0) uses a graph autoencoder with a graph attention network to generate low-dimensional cell representations of ST data and uses two decoder networks to reconstruct both the spatial neighbor graph and gene expression profiles. For our analysis, SPACE was trained with 100 epochs (instead of the default 5,000), as this was sufficient to achieve stable loss.

SEDR. SEDR (v0.0.1)⁴⁶ reconstructs the spatial neighbor graph and gene expression profile by jointly training a self-supervised deep autoencoder and a variational graph convolutional autoencoder. To ensure fair comparison with Spatial EcoTyper, spatial graphs were constructed with a 50 μm radius (dmax=50, mode=“Rad”).

BANKSY. BANKSY (v0.99.13) employs a spatial feature augmentation approach by concatenating each cell's expression profile with the average expression of its spatial neighbors. It uses a high weight (λ) to neighbor features in cell embedding to enable unsupervised clustering of tissue domains. We set λ=0.8, recommended from the tutorial, for the benchmarking analyses.

SpatialPCA. SpatialPCA (v1.3.0) is a dimension reduction method that explicitly incorporates physical distances between cells to learn a low-dimensional representation of gene expression data.

STAGATE. STAGATE (v1.0.0) implements a graph attention autoencoder that encodes both gene expression and the spatial neighbor network to generate a low-dimensional embedding of the ST data. The embedding was used to decode gene expression profiles and identify tissue domains. Spatial graphs were constructed with a 50 μm radius by setting rad_cutoff to 50, to align with the analyses using other methods.

UTAG. UTAG (v0.1.1), initially designed for single-cell proteomics, can also be applied to single cell ST data. It uses message passing (linear algebra operations) to combine gene expression and physical distance matrices. The resulting matrix is clustered to identify tissue domains. For consistency with other methods, spatial graphs were constructed with a 50 μm radius by setting max_dist to 50.

Multicellular Community Detection from Single Samples

The first benchmarking experiment evaluated the above methods for their ability to identify multicellular communities within single samples. Nine MERSCOPE samples from the SE discovery and validation cohorts were analyzed in this experiment. Due to memory constraints of SEDR and SPACE, a quadrant of each ST sample was selected for analysis, focusing on areas with minimal tissue fragmentation and high cell type diversity as determined qualitatively. Spatial clusters identified within each tissue subset were evaluated using three metrics: cell type mixing, spatial co-localization, and mean silhouette width (see “Metrics” above). To quantify overall performance, the three metrics were separately normalized across methods into a common rank space, then combined into a composite score for each method by geometric mean.

### Integrative Identification of Conserved Community Structure

The second benchmarking experiment assessed methods for their ability to identify conserved spatial ecotypes across samples. For this experiment, we used two melanoma samples profiled with MERSCOPE (‘Melanoma 1’ and ‘Melanoma 2’), and included methods supporting data integration: Spatial EcoTyper, BANKSY, SEDR, STAGATE, CellCharter, and UTAG. The five published methods applied known batch correction techniques, with BANKSY, SEDR, and STAGATE using Harmony, CellCharter using scVI, and UTAG using Combat. Notably, Harmony and scVI were designed for scRNA-seq data integration, while Combat was originally developed for bulk gene expression data. Due to memory constraints of SEDR and SPACE, a quadrant of each sample was selected as described above. Integration performance was assessed using two metrics: cell type mixing and sample mixing (see “Metrics” above). The two scores were combined for each method using a rank-based approach as described above for “Multicellular community detection from single samples”.

### Profiling SEs and Associated Cell States

### Association Between SEs and Carcinoma Ecotypes

To study the relationship between SEs and previously defined carcinoma ecotypes (CEs)⁴⁰, SEs and CEs were recovered from the same scRNA-seq data across ten cancer types using published⁴⁰ and SE-specific recovery methods respectively. The fraction of cells within each SE i that were also assigned to CE j was computed for each dataset, resulting in an overlap matrix 0 with rows representing nine SEs and columns representing nine CEs (excluding CE7 due to its low validation rate in the previous study⁴⁰). To control for potential biases from different abundances, permutation experiments were performed by shuffling the cell state assignments 10,000 times. In each iteration, the matrix O was recomputed, yielding,

$${O}_{ij}^{rand} .$$

The matrix O was then normalized using the mean and variance of

$${O}_{ij}^{rand}$$

(as described in “Cell state co-localization analysis”), producing a normalized matrix O′, where Oᵢⱼ′ represents the overlap index between SE i and CE j. Lastly, the overlap indexes across the ten cancer types were aggregated using Stouffer's approach.

### Identification of SE Cell State Markers

To identify SE-specific cell state markers, scRNA-seq data from ten cancer types were used, with all cells grouped into SE-specific cell states or the ‘NonSE’ null class. The scRNA-seq data were normalized as described in the “differential expression in tumor and stroma” section above. DE analysis was then performed by comparing each cell state with all other cells of the same cell type using the wilcoxauc function from the presto package (v1.0.0). To identify markers conserved across cancer types, gene-level mean LFCs were extracted from each cancer type and then aggregated across the ten cancer types by median. Each gene was assigned to the cell state in which it had the highest pan-cancer LFC. The top 30 genes exhibiting the highest pan-cancer LFC within each cell state were then selected as SE-specific cell state markers. Due to the limited number of NK cells in scRNA-seq data, NK cell state markers were directly derived from discovery MERSCOPE data using the same approach.

Biological Pathways Associated with SEs

Using the bulk deconvolution model, we inferred SE abundances from 7,076 TCGA tumors across 17 cancer types, including melanoma and 16 carcinomas. The SE deconvolution was performed separately for each cancer type using TPM data obtained from the TCGA PanCanAtlas.

To study the biological pathways associated with each SE, GSVA (v1.46.0) was applied to assess the activity of canonical pathways (CP), biological processes (BP), and Hallmark gene sets (H) obtained from MSigDB. The associations between these pathways and each SE were evaluated by Spearman correlation between GSVA scores and SE abundance. These correlations were computed within each cancer type and then averaged across the 17 cancer types.

Pathways specifically associated with each SE were selected using the following criteria: (i) pathway i has the highest correlation with SE j; (ii) the correlation between pathway i and SE j is higher than 0.15; (iii) the correlation between pathway i and SE j is at least 0.01 higher than the correlation between pathway i and other SEs.

### Survival Association of SEs

We investigated the association of SEs with patient overall survival using TCGA data obtained from the PanCanAtlas. A Cox regression analysis was conducted for each SE to examine the association between SE abundance and patient overall survival, adjusting for age and sex, using the survival R package (v3.6.4). This analysis was conducted separately for each cancer type. To determine the pan-cancer survival associations of SEs, a meta-analysis was performed by combining p-values from all 17 cancer types using Stouffer's method.

### Association Between SEs and Immunotherapy Response

We collected publicly available bulk tumor RNA-seq data from melanoma and carcinoma patients treated with immune checkpoint inhibitors, including anti-PD1, anti-PD-L1, or combinations of anti-PD1 and anti-CTLA-4 therapies, after tumor sample collection. All patients were grouped into responders (partial or complete response) and non-responders (stable or progressive disease) based on collected clinical information. To ensure robust analysis, patients who received prior immunotherapy or chemotherapy were separated into independent datasets. A minimum of five responders and five non-responders was required for each dataset, resulting in 1,249 total patients from 14 datasets from 12 studies, representing five cancer types (melanoma and four carcinoma types). All expression data were normalized to TPM before analysis.

Using the SE deconvolution model, we predicted SE abundances across tumors in each dataset. Additionally, we evaluated the activity of publicly available transcriptional features associated with immunotherapy response⁶⁶, including carcinoma ecotypes T cell dysfunction, T cell exclusion, microsatellite instability (MSI), Tumor Immune Dysfunction and Exclusion (TIDE), immune resistance signatures⁶⁸, IMPRES, TLS signatures, cytolytic score (GZMA and PRF1), MHC-I signature (HLA-A, HLA-B, HLA-C, B2M, CASP8), PD-L1 (CD274), 18-gene inflammatory signatures, combined tumor and immune signals (MAP4K and TBX3), an M1 macrophage signature, and an IFN-γ signature. The activity of carcinoma ecotypes from Luca et al., T cell dysfunction, exclusion, MSI, and TIDE from Jiang et al., immune resistance signatures from Jerby-Arnon et al., and IMPRES from Auslander et al. was evaluated using their respective algorithms with default settings. For the remaining features, average gene expression was computed using log₂ TPM data.

The association between each feature and ICI response was assessed using a z-score derived from a two-sided Wilcoxon rank-sum test, within each dataset. These z-scores were then combined across datasets using Liptak's method⁷⁶, weighted by the square root of sample sizes. The resulting combined z-scores were converted to two-sided p-values. Data from all four cancer types were included, whereas the comparison was restricted to melanoma datasets, as plasma cfDNA data were only available for melanoma patients.

### Liquid EcoTyper Framework

Current analyses of the tumor microenvironment rely on invasive solid tumor biopsies, which are prone to sampling bias and generally restricted to a single diagnostic biopsy. This limitation hinders the application of SEs as biomarkers in clinical settings. To address these challenges, we developed Liquid EcoTyper, a novel machine learning framework for noninvasive profiling of SEs using plasma cfDNA methylation profiles.

Liquid EcoTyper is built around a CpG Set Binary Network (CSBN), in which informative CpG sets and associated weights are learned simultaneously within a unified framework to enable multivariate prediction of SE levels. This CSBN approach draws on the Gene Set Binary Network model originally introduced for predicting cellular developmental potential from single-cell RNA-seq data. Notably, sample methylation profiles are encoded at the CpG set level for model inference, analogous to gene sets in the previous study. This representation improves robustness to batch effects and technical dropout in methylation sequencing data, while enhancing generalizability across data types, including both tumor and plasma methylation profiles.

### Network Architecture

### Input and Output

As input, the model takes an S×N matrix X containing the preprocessed methylation levels of N CpGs over S samples (see “Methylation data pre-processing”). At evaluation, the model yields an S×L matrix Ŷ containing the predicted levels of L classes over S samples. Model classes include SEs, NonSE (see “Recovery of SE-specific cell states” above), and a “background” class representing DNA not derived from the tumor compartment.

### CpG set Binary Network

Model input X is passed to a single core binary module in which M CpG sets are learned in binary N×M matrix W^{B}. As described previously⁷⁷, W^{B} has a continuous equivalent W used during training for model initialization and backpropagation that undergoes binarization at each forward pass. Here, CpG sets encoded within W^{B} are scored simply by mean of normalized methylation values over the selected CpGs per sample:

$$S := Score ( X , {W}^{B} ) = k ∘ {XW}^{B} ∈ {ℝ}^{S \times M}$$

where k denotes the scaling coefficient vector of length M containing the reciprocal of the number of CpGs selected, i.e. assigned nonzero weight, per set or column of W^{B}, and ∘ denotes entrywise multiplication. Scores are standardized across cells via batch normalization, yielding S×M score matrix S^{norm}.

### Prediction Layer

The CpG set scores encoded in S^{norm} are then passed through a linear layer to produce S×L matrix Q. This matrix is then transformed using the sigmoid function σ and rescaled to yield the final output prediction Ŷ:

$$P = σ ( Q ) + 0.001 ∈ {ℝ}^{S \times L} , \hat{Y} = l ∘ P$$

where I denotes the scaling coefficient vector of length S containing the reciprocal of the column sums of P.

### Model Training

The Liquid EcoTyper model was implemented and trained using PyTorch 2.2.0. In practice, the model feature space is of size N=38,431 CpGs, and the CpG set binary module included M=400 CpG sets.

### Loss Function

For model training we define a custom loss function incorporating both the mean cross-SE Pearson correlation per sample and the mean cross-sample Pearson correlation per SE. This loss function prioritizes robust maintenance of linear relationships, particularly of SE levels across samples—essential for clinical utility—while discouraging overfitting to training values. In detail, we define prediction loss as follows:

$$PredLoss ( \hat{Y} , Y ) = 1 - \frac{1}{S} {\sum }_{i = 1}^{S} Pearson ( {\hat{Y}}_{i}^{T} , {Y}_{i}^{T} ) + 1 - \frac{1}{L} {\sum }_{j = 1}^{L} Pearson ( {\hat{Y}}_{j} , {Y}_{j} )$$

where Ŷ and Y denote predicted and true sample level matrices, respectively, and subscripts indicate matrix columns. Along with the prediction loss, we also include a term penalizing CpG set size as described previously⁷⁴, designed to provide additional regularization to the model. Full model loss is then computed as

$$Loss ( \hat{Y} , Y , {W}^{B} ) = PredLoss ( \hat{Y} , Y ) + {‖ \frac{1}{M} ( {W}^{B} ) {( {W}^{B} )}^{T} ∘ I ‖}_{F}$$

where I denotes the M×M identity matrix and ∥⋅∥_{F} denotes the Frobenius norm.

### Model Regularization

To support robustness to technical dropout in EM-seq data, dropout was applied to model input matrix X during training at a rate of 0.5.

### Model Initialization and Updates

Model weights were given default initialization except binary weight matrix W, which here was initialized with values sampled from the Gaussian distribution with mean μ=−0.125 and standard deviation σ=0.055 to give a highly sparse initial matrix targeting 400-500 CpGs initially selected per set. At each iteration, model parameters were updated using PyTorch's NAdam optimizer with learning rate lr=0.001 and cross-epoch gradient accumulation given the stabilizing role of inertia in training binary neural networks.

### Model Training, Evaluation, and Stopping

Models were trained over ten random splits (80% training, 20% validation) of the simulated training cohort (see “Simulation of plasma cfDNA with tumor contribution”). Each model was trained for 40 epochs, with model performance by epoch evaluated over training and validation sets using the PredLoss function described above (“Loss function”). Performance on the validation split was used for early stopping. Final model weights were selected corresponding to the epoch yielding best validation performance.

### Model Ensembling

For all evaluations outside of the training framework, the outputs of all 10 models (from 10 random folds) are averaged to yield a single ensembled prediction matrix.

### Initial Feature Selection

To prepare a reduced model feature space for efficient training, informative CpGs per SE were selected from the TCGA melanoma cohort, which includes paired methylation and bulk RNA-seq profiles (n=461 tumors) obtained from the TCGA PanCanAtlas (https://gdc.cancer.gov/about-data/publications/pancanatlas). First, the set of CpGs was reduced to those detected across all TCGA melanoma tumors. For each SE, we then performed differential methylation analysis within the training cohort to identify differentially methylated CpGs. Specifically, for each SE class, we grouped samples with imputed SE abundance from paired bulk RNA-seq data above the 75th or below the 25th quantiles. We then assessed each CpG for differential methylation between groups. We computed the significance of group difference by the Wilcoxon rank sum test and the magnitude by absolute value of the difference of group means, Δ=|μ₇₅−μ₂₅|, selecting CpGs satisfying p<0.05 and Δ>0.1. If more than 5,000 CpGs met both criteria for an SE, we selected the top 5,000 by differential methylation magnitude. To ensure adequate features for SE recovery from methylation data, we required a minimum of 1,000 informative CpGs per SE. In practice, ecotype SE6 did not meet this criterion and was removed for all methylation analyses. In total, this yielded a feature space of 38,431 CpGs.

### Methylation Data Pre-Processing

### Mapping EM-seq CpGs to HM450K Probe IDs

Methylation observations from EM-seq data, given in terms of CpG locations (chromosome, start, and end coordinates), were matched to HM450 probe IDs. In short, the manifest was reduced to entries with defined values for CpG locations as well as HM450 probe ID. Any duplicate entries per CpG location were subsequently dropped, yielding a one-to-one mapping.

### Normalization and Imputation

Data were subset to model features with methylation values subtracted from one and normalized to zero mean and unit variance over model CpGs per sample. Missing values were then imputed per CpG by dataset mean when defined and replaced by zeros otherwise.

Simulation of Plasma cfDNA with Tumor Contribution

Due to the limited availability of paired tumor and plasma cfDNA data, we simulated a training dataset by combining plasma cfDNA and tumor methylation profiles. Paired bulk RNA-seq data from these tumor samples enabled the recovery of tissue SE levels through bulk deconvolution as previously described (see “Deconvolution of SEs from bulk RNA-seq”). We designed simulated mixtures to contain both “background” cfDNA and tumor tissue contributions, with background compartment derived from plasma cfDNA profiled from Cohort 2 healthy individuals (n=23 total; partitioned into n=18 for training cohort and n=5 for test cohort), and tumor compartment derived from the TCGA melanoma methylation cohort (n=461 total; partitioned into n=346 for training cohort and n=115 for test cohort).

Generating Additional Background cfDNA Profiles

To expand the pool of healthy cfDNA profiles, we prepared new profiles from the samples of each cohort separately to reach the same number of samples available from tumor tissue. For each new profile, we randomly selected three samples from the appropriate cohort (training or test), perturbing the methylation profiles of each sample with noise to reduce collinearity across new mixtures, then combining according to randomly generated fractions.

In detail, fractional composition was generated by sampling from the unit interval uniformly at random once per sample, then rescaling the three values to sum to one, yielding mixing fractions φ₁, φ₂, φ₃. Noise perturbations were applied multiplicatively to raw methylation values, with noise level parameters v₁,v₂,v₃ generated by sampling from the normal distribution. Given sample methylation profiles M₁,M₂,M₃, perturbed profiles \hat{M}₁, \hat{M}₂, \hat{M}₃, were computed as follows:

$${\hat{M}}_{i} = min ( ( 1 + {sv}_{i} ) {M}_{i} , 1 )$$

where s denotes a scaling parameter, set here to s=0.02, and min operates elementwise to cap resulting methylation values at 1. With these mixing parameters defined, the resulting new healthy cfDNA profile M_{new} is given by:

$${M}_{new} = {\sum }_{i = 1}^{3} {ϕ}_{i} {\hat{M}}_{i} .$$

### Combination of Background and Tumor Compartments

With the resulting background cfDNA profiles now matching the tumor tissue profiles in number, we generate the final sets of simulated samples by matching background and tumor profiles one-to-one within training and test cohorts. For each simulated sample, we generate mixing fractions φ_{T} and φ_{B} of tumor and background by sampling φ_{B} uniformly at random from a desired range, then computing φ_{T}=1−φ_{B} as the complement. This range was selected here as [0.2, 0.6] to weight fractions in favor of tumor contribution while preserving substantial background for regularization in order to support model extensibility to both tumor tissue and plasma cfDNA methylation profiles. The resulting simulated samples are then constructed as

$${M}_{new} = {ϕ}_{T} {M}_{T} + {ϕ}_{B} {M}_{B} ,$$

where M_{T} denotes the raw methylation profile of the selected tumor tissue sample and M_{B} denotes the raw methylation profile of the selected background profile, newly generated as described above.

### Ground Truth Composition of Simulated Samples

For each simulated sample, levels of SEs were recovered by applying Spatial EcoTyper to the methylation profiles of each tumor sample as previously described (“Deconvolution of SEs from bulk RNA-seq”). As we excluded SE6 from Liquid EcoTyper analyses due to inadequate feature support (“Initial feature selection”), we removed it from predictions and rescaled the remaining outputs to sum to one. Ground truth SE and NonSE levels were then defined as the resulting predictions scaled by φ_{T}, with ground truth background level given by φ_{B}.

### Performance Assessment of Liquid EcoTyper

### On Held-Out Data

We first tested Liquid EcoTyper's ability to recover ground truth levels of SEs from plasma and tumor methylation profiles by application to the held-out test cohort of simulated data (n=115; “Simulation of plasma cfDNA with tumor contribution”). Performance was assessed by Spearman correlation between ground truth and predicted levels for each SE.

On Paired Tumor and Plasma from Melanoma Patients

To further validate the generalizability of Liquid EcoTyper to liquid biopsy data, we generated paired EM-seq data from 10 melanoma patients (Cohort 2), with paired tumor and plasma available for all patients, and with additional PBMC profiling available for seven of ten. Applying Liquid EcoTyper, we estimated SE levels from these samples as described previously. We then evaluated the consistency of SE levels between tumor and plasma samples, as well as between tumor and PBMC samples and plasma and PBMC samples to show signal specificity. The consistency was quantified by Spearman correlation for each SE (FIG. 5b,c).

### Association Between Liquid SE Levels and Immunotherapy Response

To evaluate the clinical relevance of SE levels in plasma cfDNA, we applied Liquid EcoTyper to infer SE levels in 79 pre-treatment plasma samples from melanoma patients in Cohort 3 and 10 plasma samples from Cohort 2. The association between each SE and immunotherapy response was assessed using a two-sided Wilcoxon rank-sum test, comparing patients with DCB to those with NDB in each dataset.

### Survival Analysis of Liquid SE Levels

We analyzed the association between plasma cfDNA SE levels and patient survival by dichotomizing patients based on the median inferred level of each SE in Cohort 3. Differences in overall survival and progression-free survival between the two groups were assessed using the log-rank test with the survival (v3.6.4) R package. Additionally, multivariable Cox regression was conducted for SE7, SE8, and SE4, adjusting for melanoma subtype, BRAF mutation status, sex, age, treatment, and ctDNA levels.

What is claimed is:

1. A method for identifying tissue microenvironments, the method comprising:
acquiring spatial omics data of a plurality of solid tissue samples, wherein the spatial transcriptomics comprises for each cell of each solid tissue sample: a location, a cell type, and an omic data profile, wherein the spatial omics data comprises a number of cell types, wherein the number of cell types is greater than two;
for each solid tissue sample, dividing the spatial omics data into an array of spatial microregions comprising at least ten microregions, wherein each microregion is defined by a contiguous spatial area;
for each cell type, determining a cell-type-specific microregion omic data profile, wherein the cell-type-specific microregion omic data profile is an aggregate of omic data profiles of cells of the same cell type within the microregion;
for each cell type, generating a cell-type covariance matrix of cell-type-specific microregion omic data profiles across the array of spatial microregions;
integrating the cell-type covariance matrices to yield a sample-level covariance matrix of microregion omic data profiles across the cell types; and
identifying sample-level microenvironments within the sample-level covariance matrix, wherein each sample-level microenvironment is based on covariance of the microregion omic data profiles.

2. The method of claim 1, wherein integrating the cell-type covariance matrices comprises fusing the cell-type covariance matrices via a similarity network fusion method.

3. The method of claim 1 or 2, wherein identifying sample-level microenvironments comprises grouping the microregion omic data profiles via a clustering method.

4. The method of any one of claims 1-3 further comprising:
repeating the steps of claim 1 for a plurality of samples such that a total number samples is greater than two such that sample-level microenvironments are identified in each sample;
for each sample-level microenvironment, determine a cell-type-specific microenvironment omic data profile for each cell type of the microenvironment;
for each cell type, generating a multi-sample cell-type covariance matrix of cell-type-specific microenvironment omic data profiles across the samples;
integrating the multi-sample cell-type covariance matrices to yield a multi-sample-level covariance matrix of cell-type-specific microenvironment omic data profiles across the cell types; and
identifying conserved microenvironments within the multi-sample-level covariance matrix, wherein each conserved microenvironment is based on covariance of the microenvironment omic data profiles across the samples.

5. The method of claim 4, wherein the plurality of solid tissue samples comprises a plurality of tissue types.

6. The method of claim 5, wherein the plurality of tissue types comprises a plurality of cancer types.

7. The method of any one of claims 4-6, wherein the plurality of solid tissue samples comprises samples extracted from a plurality of individuals.

8. The method of any one of claims 4-7, wherein integrating the cell-type covariance matrices comprises fusing the multi-sample cell-type covariance matrices via a similarity network fusion method.

9. The method of any one of claims 4-8, wherein identifying sample-level microenvironments comprises grouping the microregion omic data profiles via a clustering method.

10. The method of any one of claims 4-9 further comprising:
training a matrix factorization model to deconvolve the plurality of conserved tissue microenvironments from bulk biomolecule sequencing data of a solid tissue.

11. The method of claim 10 further comprising:
determining that one or more conserved tissue microenvironments is indicative of a clinical phenotype;
obtaining bulk biomolecule sequencing data of a tissue sample derived from a patient;
deconvolving the bulk biomolecule sequencing data and determining that the tissue sample indicates the clinical phenotype based on an abundance of the one or more conserved tissue microenvironments indicative of the clinical phenotype; and
performing a clinical action upon the patient based on the indication of the clinical phenotype.

12. The method of claim 11, wherein the clinical phenotype is a medical condition capable of being treated by a therapeutic; wherein performing the clinical action comprises:
administering the therapeutic to the patient.

13. The method of claim 12, wherein the medical condition is cancer and the therapeutic is a chemotherapeutic, a targeted therapeutic, an immune checkpoint inhibitor, or an immunotherapeutic.

14. The method of claim 11, wherein the clinical phenotype is positive responsiveness to a therapeutic for a treatment of a medical condition; wherein performing the clinical action comprises:
administering the therapeutic to the patient.

15. The method of claim 14, wherein the medical condition is cancer and the therapeutic is a check point inhibitor.

16. The method of any one of claims 4-9 further comprising:
training a binary neural network to detect abundance of one or more conserved tissue microenvironments within a cell-free biomolecule sequencing result.

17. The method of claim 16, further comprising:
determining that one or more conserved tissue microenvironments is indicative of a clinical phenotype;
collecting a cell-free biomolecule sample form a patient;
sequencing the cell-free biomolecule sample via deep sequencing to obtain a cell-free biomolecule sequencing data result;
entering the cell-free biomolecule sequencing data result into the trained binary neural network and inferring that the cell-free biomolecule sample indicates the clinical phenotype based on an abundance of the one or more conserved tissue microenvironments; and
performing a clinical action upon the patient based on the indication of the clinical phenotype.

18. The method of claim 17, wherein the clinical phenotype is a medical condition capable of being treated by a therapeutic; wherein performing the clinical action comprises:
administering the therapeutic to the patient.

19. The method of claim 18, wherein the medical condition is cancer and the therapeutic is a chemotherapeutic, a targeted therapeutic, an immune checkpoint inhibitor, or an immunotherapeutic.

20. The method of claim 17, wherein the clinical phenotype is positive responsiveness to a therapeutic for a treatment of a medical condition; wherein performing the clinical action comprises:
administering the therapeutic to the patient.

21. The method of claim 20, wherein the medical condition is cancer and the therapeutic is a check point inhibitor.

22. The method of claim 16, further comprising:
determining that one or more conserved tissue microenvironments is indicative of a clinical phenotype; and
repeatedly over a period of time:
collecting a cell-free biomolecule sample form a patient;
sequencing the cell-free biomolecule sample via deep sequencing to obtain a cell-free biomolecule sequencing data result; and
entering the cell-free biomolecule sequencing data result into the trained binary neural network to monitor whether the cell-free biomolecule sample indicates the clinical phenotype based on an abundance of the one or more conserved tissue microenvironments.

23. The method of claim 22, wherein the clinical phenotype is progression of a medical condition.

24. The method of claim 23 further comprising:
when the progression of the medical condition exceeds a threshold, modify a treatment regimen of a therapeutic; and
administer the therapeutic to the patient in accordance with the modified treatment regimen.

25. The method of claim 24, wherein the medical condition is presence of minimal residual disease of cancer and the modified treatment regimen is reinitiating administration of the therapeutic, wherein the therapeutic is a chemotherapeutic, a targeted therapeutic, an immune checkpoint inhibitor, or an immunotherapeutic.

26. The method of claim 22, wherein the clinical phenotype is response to a treatment regimen of a therapeutic.

27. The method of claim 26 further comprising:
when the response to therapy indicates a need to modify the treatment regimen of the therapeutic, modifying the treatment regimen of the therapeutic; and
administer the therapeutic to the patient in accordance with the modified treatment regimen.

28. The method of claim 27, wherein the response to therapy is a lack of response to the treatment regimen of the therapeutic or an undesired side effect of resulting from the treatment regimen of the therapeutic, wherein modifying the treatment regimen of the therapeutic comprises administering an alternative therapeutic to the patient.

29. The method of claim 27 or 28, wherein the therapeutic and the alternative therapeutic are each individually a chemotherapeutic, a targeted therapeutic, an immune checkpoint inhibitor, or an immunotherapeutic for the treatment of cancer.

30. The method of claim 27, wherein the response to therapy is a pathologic complete response of a cancer, wherein modifying the treatment regimen of the therapeutic comprises terminating administration of the therapeutic to the patient, wherein the therapeutic is a chemotherapeutic, a targeted therapeutic, an immune checkpoint inhibitor, or an immunotherapeutic.

31. The method of any one of claims 4-30 further comprising:
identifying one or more omic-data markers within one or more of the conserved microenvironments.

32. The method of claim 31 further comprising:
acquiring single-cell-omic data of a number of solid tissue samples, wherein the number of solid tissue samples is greater than two;
assigning cells of the single-cell-omic data to one conserved microenvironment of the identified conserved microenvironments, wherein each cell is assigned based its omic data grouping with the conserved microenvironments via a clustering technique;
for each conserved microenvironment, aggregating the omic data of the cells assigned to yield a conserved-microenvironment omic data profile;
perform differential analysis comparing each conserved-microenvironment omic data profile with all other conserved-microenvironment omic data profiles;
for each putative marker, extract a marker-level log-fold change from the differential analysis;
assigning each putative marker to one conserved-microenvironment omic data profile; wherein each putative marker is assigned based on which conserved-microenvironment omic data profile had the putative marker's highest log-fold change as determined by the differential analysis; and
assigning one or more markers for each conserved-microenvironment omic data profile, wherein each assigned marker has a log-fold change that is one of the highest of the putative markers assigned to that conserved-microenvironment omic data profile.

33. The method of claim 32, wherein each solid tissue sample is a cancer type, wherein the number of solid tissue samples comprises at least three cancer types.

34. The method of claim 32 or 33 further comprising:
determining that one or more conserved tissue microenvironments is indicative of a clinical phenotype;
acquiring a omic-data sequencing result of a patient solid tissue sample; and
assessing the patient's omic-data sequencing result to determine whether the patient's solid tissue sample comprises an abundance of the one or more conserved tissue microenvironments that is indicative of the clinical phenotype; wherein the presence of the one or more markers assigned for each conserved-microenvironment omic data profile is utilized to determine the abundance of the one or more conserved tissue microenvironments that is indicative of the clinical phenotype.

35. The method of claim 35, wherein the clinical phenotype is responsiveness to a therapeutic for treating cancer; the method further comprising:
determining that the patient's solid tissue sample comprises an abundance of the one or more conserved tissue microenvironments that is indicative of responding to the therapeutic for treating cancer; and
administering the therapeutic for treating cancer to the patient.

36. The method of claim 35, wherein the therapeutic is a chemotherapeutic, a targeted therapeutic, an immune checkpoint inhibitor, or an immunotherapeutic.

37. The method of any one of claims 1-36, wherein the omic is one of: transcriptomic, methylomic, epigenomic, or proteomic.

38. A diagnostic method for assessing an individual for cancer, comprising:
receiving a collection of a cell-free sample of a patient; and
sequencing the cell-free sample to yield a cell-free-sample sequencing result to determine whether an abundance of one or more tumor spatial ecotypes in the patient,

39. The method of claim 38, wherein the abundance of the one or more tumor spatial ecotypes is indicative of a responsiveness to a particular treatment.

40. The method of claim 39 further comprising:
determining that the abundance of one or more tumor spatial ecotypes is present in the patient based on the cell-free-sample sequencing result; and
administering the to the individual the particular treatment.

41. The method of claim 39 further comprising:
determining that the abundance of one or more tumor spatial ecotypes is not present in the patient based on the cell-free-sample sequencing result; and
administering the to the individual a treatment that is not the particular treatment.

42. The method of any one of claims 39-41, wherein determining whether the one or more tumor spatial ecotypes is present in the patient comprises:
identifying whether a presence one or more markers in the cell-free-sample sequencing result that indicate the abundance of one or more tumor spatial ecotypes is present in the patient.

43. The method of claim 42, wherein the particular treatment comprises the administration of an immune checkpoint inhibitor, wherein the one or more markers indicates at least one of:
expression of STMN1, TUBB, TYMS or GZMB within CD8 T-cells;
expression of TNFRSF4, TNFRSF18, IL2RA, CTLA4, or FOXP3 within CD4 T-cells;
expression of CCL8 or ISG15 within macrophages;
expression of CXCL8 or MMP1 within fibroblasts; or
expression of CEACAM1 or CEBPB within endothelial cells.

44. The method of any one of claims 39-43, wherein determining whether the one or more tumor spatial ecotypes is present in the patient comprises:
entering the cell-free-sample sequencing result into a trained binary neural network to infer whether the abundance of the one or more tumor spatial ecotypes is present in the patient.

45. The method of any one of claims 39-44, wherein the one or more tumor spatial ecotypes is one or more of: Spatial Ecotype 8, Spatial Ecotype 7, and Spatial Ecotype 4.

46. The method of claim 40 or 41, wherein the particular treatment comprises administration of a chemotherapeutic, a targeted therapeutic, an immune checkpoint inhibitor, or an immunotherapeutic.

47. The method of any one of claims 38-46, wherein sequencing the cell-free sample comprises:
methylation-sequencing of cell-free DNA or RNA-sequencing of cell-free RNA.

48. The method of any one of claims 38-47, wherein the collection of the cell-free sample was collected prior to initiation of a treatment for cancer to assess for presence or progression of cancer within the patient.

49. The method of claim 48, wherein the assessment for presence or progression of cancer within the patient is periodically repeated over a time course to monitor the patient.

50. The method of any one of claims 38-47, wherein the collection of the cell-free sample was collected after to initiating a treatment of a cancer to assess responsiveness to the treatment.

51. The method of claim 50, wherein the assessment of the responsiveness to the treatment is periodically repeated over a duration of the treatment.

52. The method of any one of claims 38-47, wherein the collection of the cell-free sample was collected after completing a treatment of a cancer to assess whether minimal residual disease is present within the patient.

53. The method of claim 52, wherein the assessment of whether minimal residual disease is present within the patient is periodically repeated over a time course to monitor the patient.

54. The method of claim 53, wherein the time course is at least one year, at least five years, at least 10 years, or at least 20 years.

55. A diagnostic method for assessing an individual for cancer, comprising:
receiving a sample of a solid-tumor biopsy of a patient;
extracting nucleic acids from the solid-tumor sample; and
sequencing the nucleic acids to yield a solid-tumor-sample sequencing result to determine whether an abundance of one or more tumor spatial ecotypes in the patient, wherein the abundance of the one or more tumor spatial ecotypes is:
indicative of a responsiveness to a particular treatment, or
indicative of a pathology that can be treated by a particular treatment.

56. The method of claim 55 further comprising:
determining that the abundance of one or more tumor spatial ecotypes is present in the patient based on the solid-tumor-sample sequencing result; and
administering the to the individual the particular treatment.

57. The method of claim 55 further comprising:
determining that the abundance of one or more tumor spatial ecotypes is not present in the patient based on the solid-tumor-sample sequencing result; and
administering the to the individual a treatment that is not the particular treatment.

58. The method of claim 56 or 57, wherein the particular treatment comprises administration of a chemotherapeutic, a targeted therapeutic, an immune checkpoint inhibitor, or an immunotherapeutic.

59. The method of any one of claims 55-58, wherein determining whether the one or more tumor spatial ecotypes is present in the patient comprises:
identifying whether a presence one or more markers in the solid-tumor-sample sequencing result that indicate the abundance of one or more tumor spatial ecotypes is present in the patient.

60. The method of claim 59, wherein the particular treatment comprises the administration of an immune checkpoint inhibitor, wherein the one or more markers indicates at least one of:
expression of STMN1, TUBB, TYMS or GZMB within CD8 T-cells;
expression of TNFRSF4, TNFRSF18, IL2RA, CTLA4, or FOXP3 within CD4 T-cells;
expression of CCL8 or ISG15 within macrophages;
expression of CXCL8 or MMP1 within fibroblasts; or
expression of CEACAM1 or CEBPB within endothelial cells.

61. The method of any one of claims 55-60, wherein determining whether the one or more tumor spatial ecotypes is present in the patient comprises:
entering the solid-tumor-sample sequencing result into a matrix factorization model to deconvolve the sequencing result into abundances of tumor spatial ecotypes to determine whether the abundance of one or more tumor spatial ecotypes is present in the patient.

62. The method of any one of claims 55-61, wherein the one or more tumor spatial ecotypes is one or more of: Spatial Ecotype 8, Spatial Ecotype 7, and Spatial Ecotype 4.

63. The method of any one of claims 55-62, wherein sequencing the cell-free sample comprises:
methylation-sequencing of cellular DNA or RNA-sequencing of cellular RNA.

64. The method of any one of claims 55-63, wherein the biopsy of the solid-tumor sample was collected prior to initiation of a treatment for cancer.

65. The method of any one of claims 55-63, wherein the biopsy of the solid-tumor sample was collected after to initiating a treatment of a cancer to assess whether the treatment is to be modified.
